SEQUENCE LISTING (1 sequence)

SEQ ID NO: 1 - DNA, 109756 bp, Chlorella sorokiniana
  misc_feature 109756^1: note = Chlorella sorokiniana - chloroplast genome; note = Sequence is circular
  source 1..109756: mol_type = genomic DNA; organelle = plastid:chloroplast
aaaatagaacaggtttgaattttactgactttgtgagttttaaaactcacaaagtcatttaaaagattaaaaaaaagtaaaaagtttttttgtttttatgaatttaatttgaaatgtatctcttccccccccaaattagatttttgagactaacttggttttttcacaaatttaaaaagttttctcccaaaaacactgtttttagaaatggaaggcaaaagaactagtttctaaattgtttgatactttgatttttttaggatcaaaatacttattaatatgacctttaaaattaggcagttagtttgtaaaaaaatgatggtagttttctattcccaaaatctttgtttttctccatttcctaggcgtaaacgcactagggaaaagtttgtttttcctagaaaacggggaagtgataaatttagaaactttagagagaaaatactaaaattaaaaaaattaatcttcatgttcttcaaaaggatcacgaagtttttgtgacggtgggccaaaaccaacataaatagaataaccagtaatacttaataaaagacaccacaaaaaaatggtaaagaaaaaagctggactttccatgggtaaaaattaaaaaaataagaaaattctttagaagataatacttttttcacaatttgtttattagtttttatgaaaaaagaaaaaaattatctttcatctattcctgttttcattttaccaaaaaacaaggaaaaagttatgttaaagtattatttttcttttaatcctcaaaaatggaaatctctacaagacttcttatttgtattctaaaacttttgagaaatttaaacttactatataatagagataagacagagaaaaaagcttaaaagttttttctcgtttttacagaaagtaaaaaaaaattctttaatttcaacaattatggcaactggaactacatcaaaagtaaaatctgaagatactggaatttcaactccattaggaactttgctaaaaccattaaattctgaatatggtaaagtagcacctggctggggtacaactgttttaatgggcatttttatggctttatttgcagtttttcttgtaattattttagaaatctacaacagttcagttcttcttgatgatgtaactatgagttgggaatctttagctaaataaaaaaattttgtattcctttcctatttccattcccaaatttaaaaaatttgggaatggaaataggaaggcaaattattttaaggtagaaaaattctaaaaaaaattttttaccgtttttgaacctaaaaacagaatttaacagtttgttaaattttagagaactaaatgaaatggattaattaatttttttggttgaaaaaaacttaaatttttttccaagcacaaatttcatccaaaattttttaaattttgggatagaaagaagaaatcgtttttaacttactaaaaaagctttagtgtttaaatttcaatgaagtgaaatcagaaaatttttctttttcagttttgtagtttggggatagagagacttgaactctcacggttcaatgaaccaacggattttcagatatgaaactggactctctctttatcctcacagtaaacaacagttactttaaagaaaagattttccctgttttgttattgtattaggatagctcccgtcgagtctctgcacctcttaagaaaatttttcttaagttgactcaggattacttaaaatttttcattaaaaataaagtttccctgaatttgagagcattcactttaaaagtttccttttaaaggctcaaaatatgagtgaagttgtacctttaagtccgttgcgtctgccgattccgccatatccccgaaagacaacttgtattatcaatacttattctactaaaaataaaaaaaaacactagttgttttttttttctgtgtaaagtagactattcagtagtaatacaaaaaactaaaaattaaattttaaatactttaatttaatcctttaccgttcaatagtaaaggtaatgagttaaaggatttttcatttgaattttcgggtcgatgcccgagtggttaatgggggtggattgtaaatccactggctctgcctacgctggttcaaatccagctcggcccaaaataaaaattgatggtggttttgagcagaaaaagttccacctttataaaactcaagaaaataataaaaaactaatgaaagttattagggtgggcataaataaaatttcagaaagtttttaaattttgtctcagtgtttattttacatacaaatagtagatagtattgtaactaattttctcctacaaagaacatgctttttttagggtcttgagtaaaaaagtctatttagtaatgggctcacgttttttagactataaaagtctaaaaatcaatcttaagtcttttattttttttattgtaaaactgaatccatgtacttttttcaaatcattatatcattttggtttcagaattaagaaaatacgtaatgaaaaaaatattttatatttgaaaaggctttaaaaatttgcattttttgagttcaatatagttcaatctgtaaaaacttgcgaacccctaaacaaagaagtccgcaagggttctctttttgtattgaaaccaattgcttagttttgagttaatcttattgttcttgacttgcagtctttacataaagaattaaaagaaaagatgttgggataataataaataacgcagttgcaattacaccaaggatattcacttccattgaaattttactccttactttaaattaagtgtttgacactttatggtttcctcaaaaaaattttttgagagaatcatttcctttttacgattaaaacgttttcaaaaagggaaaaggttttttactccatctgaatgaagtcccttgcaaaaactagacttaaatctatcttaaatctaaaattattttctacttttctattctttttattttcaagagaaaataaaaagaaaaaaaagtagaaaataattttagagcggaatgtcttagatttttatcaagagaattttgtcttcttttaaatattgtatcgtaaaaacgaaaaaacaaaaaaggttttcaaaacaatatgttttacaagttttctttcggaaagggggggattcgaacccccggtagtctcgcaactacgtcggttttcaaaaccgatgcattcaaccgctctgccacctttccacgagccgaaccgttaagctattaatacacctcaaacggaaaactctatgtgaagttttcgtgattcttctttagtttaaactgaaaacctcttttaggcaactagcgctttctgacagccctcttttaattttaaaaaataactttaaaaaacagaaaatttaaaaatagactcaacataaatttttgaaatctattctagacagaaagaattcttttcttacaataaactgtagacttttacagaaattaccaaatgcaaataattgataatctaaataacaaatataaagatttgtactttaactgagtttaaaattgtttatttaaagaaatatgagtttagcataaaacttataaaattttataagcgggtaacgcggatcgaacgcgcgacatgcaccttggcaaggtgccgctctaccactgagctatacccgcactctttgtatttatttacattaaactgaaccaacaactaggtcttttgtttttctgccatagacaaaaaagtgttgtttatattttttcttttattttaacagagtcattaattttttgcaatctcaaaatttaaaaataagtgatgtaaaaaactgtgtgttgtgaagttttttatattgtttaattgtttagtaaatttgtattttcgctctagattaattcaaaatacttttctcttgacaacaagtcttccttaatttagactatgtctaaatttaggtaaaaagcggggtagagcagtctggtagctcgtaaggctcataaccttaaggtcgtaggttcgaatcctacctccgctctgattcttgacaaaaagaatcacttcctgtagtatactaaatatcaccttaaactttaaggtaaaggcccccatcgtctagaggcctaggacatctccctttcacggaggaaacggggattcgaattcccctgggggtagacaagtttgtaacaacacactacaataaaagcataatctattaaaaacagaaatttcgtgcaaaccttttttctttgtgctatgaaatttcgattcaaaacgagctcattccaaaaatggaatttttgggaagggtctccaaatgaaactggtttgaaaaaaaatacattttcatctcttctcaaaaacattgttttttggaagggctgaaaatagatcactgggtttttcgttatacccagtttactacattcttaagatcaaagaatacaataagttaaaaaatcagttaactctttttctaacagtctaaaagagttaatattgattgttttctataaaaaaaggttattcgcaaaaaggattgctgtttatgactcgagtaaaacgaggaaatgtggctcgaaaacggagaaagcaggtgctaaatttagcatcagggtttcgaggttcatcatcaaaactctttagaacagcgcagcaacaaacaatgaaatctttaagttattcataccgcgatcgtcaacaaaaaaaacgtgagttttgcgggctttggattactcgaattaatgctgcagttcgttcgcacggattgaattacaacgaatttcgtcatcacttaaaaaaatcaggaattcagcttaaccgcaaagtgctgagtcaagttgctcttcgtgatcaacaagcttttgctgaattagttgaattagtaaaaaaataaacagagaaactttttcctaaaagaattttttttggataagttcgaattcaagtcgagaattcaactagaactttttatttttaaaaaaattagttggattttagtcctctcccaaaaaatatatttttgttattaacttttaaatctttaaagaatacgaaccataaagagtaatcttttgggttctaaaaaagtttttttggaaaggcattaattgtatttattaaaaataaaaagtagtttctttttctaaaaggaggtgaaaaccataatggctggaactcagcaaaaacgaaaaagtttaaaatcatttaaaaatcgacgaaaagcaattcctgttgtaattcctaaaaaacaacaagcgattggtaccactacaacacaaccagctcctcgatttctaattgattataaaaatactaaattattagtgaaatttattagtccacaaggaaaaatattatctcgaagagcgacgggattaactgcaaaacaacaacgtgtcatgtcaaatgccatcaaacgagcccgaatgggtggtttattaccgtttgtaaactatgatattgcaggaaataagtaaattttaagaaaagagataatatttttgattttctaaaaatgtggccagtttttttttaaaaacgtcaagttctagagtaaagggaggattttaagttaactttctagctctgaaaatttgatttttattggtaaatttgaaaaattttggacaaatggatttcctttttttcaaaaacttttttttgagattatttgaaatatttagaataacactaccatatttcagttttttttgagaaaatataagaaatatcaattttcacgccttgtaggacttgaacccacggcatcaggttttggaaacctgcgttctaccaactgaactaaaggcgttcgggatgacaggattcgaacctgcgacctcctgttcccaaaacaggagcgctaccaaactgcgctacatcccgagagatcatttctctttttagtctacacaacacaattctttttttcaagtgtttgtttaattctttccttttttattagtatttccaaaaaagtttttttaaatttgagaggtttttaaaaaaaattttagtattcgttattattaatttttcttttcttcttttaccaaaaattctatttttggggttgggctagattaagtcaattttttggaaaaaacaaaaatgttaggtaggaaaaagagtcgatgattctattgaaaattgacaagaaaatatcgaaaaaagaaacgggagagttttccatgaaagattttacaacatatttatcaacagcacctgttttaacattgttaagtgtaactgtagttgctgggcttttaattgaaatcaatagatttttccctgacgctttaattgctgctttttaaaacaaactcttttaaaaccgacattatttagctcatcttagttctctgttccctttttttccaaaaattttttaaatttttggaaaaaaagggaacagagaactaagattcaagtaaaaaatgtaaaacccatgaaaaaaaatttttagtttagtgtgacacattttagttatgcctacgattcaacaattagtacgatctgctcgaactcgtctttcgaaaaaaacaaaatcgcctgctttaaagagttgcccacaacgacgtggtgtttgtactcgagtttacacaacaacaccaaaaaaaccaaactctgctttacgtaaagttgctcgtgtgcgacttacatctggttttgaagtgacagcttatattccaggtattggtcataatttacaagaacactctgttgtattagttcgtggtggtcgagtaaaagatttacctggagttcgttatcatattgttcgtggaacattagattctgcaggtgtaaaagatcgttctcaaagccgttcgaaatacggcgttaaacgtccaaaataaagaaaaaattttattgaatttatgtctcgtcgacgtactccaaaaaaacgtattgtaatgccagatccagtctatgatagtcgtttagttgaacttttagttcgacaactaatgcgagaaggcaaaaaatcattagcatatcgaatttgctatgaaagtatgaatcaagtagctgatacaactcaacaagatccgcttgttgttcttgaacaagctattcgaaatgctactccacttgtagaagttaaagctcgtcgtattggtggatcaacttatcaagttcctttagaggttgctcctgaacggggaacagctcttgcaattcgctggattctttctgcttgtcggaataagtctggccgaccaatggcaatgaaattagcaagcgaacttttagatgctgcaaaaaatgcaggacttgctgttagaaaacgagatgaagttcataaaatggcagaagctaataaagcttttgcaaaatatcgtttctaaatcaatcttttttttttttttttaatttaaaatttacacttctttcccaaaacctttgtttaccctcaaccaaatttttggggaacagcttaaaccttattttttttccaaggtagggagagtaaaactatttcttttgcttaaaaaaaaaaataaaagttgagaaaagaaataaaaaagaaaaattatcaaaatcaaagattttactaaacaggcagttaaccctgtcccaaaactctaaatagttttgggattaactgtgagttagtccctgtctcaaaaatttcgttttgggattaaaaaaattatttactccaaatttgcaaaatttgaggaaggggttcccaaaaatgaaatttttgggtgggacaggttctattaaaaattacaaaaaggaaactttttatggcacgtgaaaaatttgaacgtataaaaccacacgtaaatattggaacaattgggcacgtagatcacggaaaaactacattaacagctgcaattactatggctttagctgctcgcggtggcgcaaaaggtcgtaaatatgatgatatcgattctgctccagaagaaaaagcacgtgggattacaattaatacagctcacgtagaatacgaaacagacaatcgtcattacgcgcacgttgattgtccagggcacgctgactatgtaaaaaacatgattacaggtgctgcgcaaatggatggtgcaattcttgttgtttctggtgctgatggtccaatgccacaaacaaaagaacatttacttttagctaaacaagtaggtgttccaaatattgttgtattcttaaataaagaagatcaagtagatgatgctgaattattagaacttgttgaacttgaaattcgagaaacattagataaatatgaatttccgggtgatgaaattccaattatcgcaggatctgctcttttagctttagaagcattaacagaaaaccctcaaattaaaccaggtgacagtaaatgggttgacaaaatttataaccttatggatcaggttgatgcttatattccaacacctgaacgtgaaacagagaaaccattcttaatggcggtagaagatgtcttttcaattactggacgtggtactgttgctacaggacgtgtagagcgtggctgtgttaaaatcggtgacactgtcgaacttgtaggtttacgtgacacaaaaacgacaacagtaactggtttagagatgttccaaaaaacattagatgaaagtgttgctggtgataacgtaggtattttacttcgtggtgttcaaaaaacagatattgaacgtggtatggttcttgcaaaaccaggaagcattacacctcacacaaaatttgaagctcaagtttatgttttaacaaaagaagagggaggtcgtcatactccatttttcccaggatatcgaccacaattttatgttcgtacaacagacgtaacaggtaaaattgaatctttccgcgcagatgacgatagcgcaacacaaatggtaatgccaggtgaccgtgtgaaaatgattgttgaacttattcaacctattgccattgaaaaaggaatgcgttttgcaattcgtgaaggtggacgtacagttggtgcaggtgtagtttctacaattgttatttaaatttgtaaacgttcggttctcctaaaacaagtttagtcatggtatctcttcctgtcccaaaaacttaggtataccttaaatttttcattttttgggaaaggacctctatttctcccaaactttgtttgggagaaatggaaacgagataaaaattgtttttgcgacaagcttagattttgtggggccgtgacaagcaaatttaaaaaaaatttagtttgatttttaaaaatacatacaagtattttttatattctcatcttgtttttgtactaaatgtttcaattttcgcgttctcaaaatccaaatataacttgttttatagattttgatactcacgttttatcaaaaaaggatttacattattctgtcgtttaattccaaaaacttcgttttttggaaaggagctaaataaaattcgtttttttaaattgaagcagttaaatattctaacaaagaaaaatatcaagctataaactgaaattagaaactatttttataacagaaagacacaagggtagctattatgactaagcttcaacaacttgtccagacaatccagtctgagatcgtaaaaacagagttaccagatattcgtgtcggagatttagtacgcattggcgtttcaattcaagaaggaaataaacaacgtgtgcaaccgtttgaaggcacagttattgcaaaacatcaagcgggtgtcaatactaccgtaacagtccgtaaaacgttacagggtgttggagtagagcgtgtatttcctgtctatgctccttgtattgcaaatgtccaaattcttcgccgagctcaggtatctcgttcaaaattgtattatttacgcaatcgaactggtaaggcaactcgattaaaagaaaaatttgaaacattacctaatgtttgggtaaaccaaaaataataacaaaagcgttttactccctgtcccaaaaacaaagtttttgggataaaatttttacaggtccattataaaaaatttttttaggaaggacaccttaaaaaactctctatttttagggaagtctttttcataacaaaatttgaaaaaaacttttggactaagtcaaaacctattgttttgaaaaaatgcaagattcttaatttctgtgcaaaaaaaatggagtgttttccgtcccaaaaatgtcatttttgggaaagtaagctcttaaaatttgagaacactaagaaacaagctccagtcttgtttttgaggcaagaaaaaaagtaagaaaatacaccatgacagcgcaagaagctattatacgttatgaagttcctggcgcgcgacgcgttggtaattatgtttggggatctttaatgtgccttggaggaattggctttttagtcactggactttcaagttattttgatattccattcttttctggactgacttctccaaccatacaatttttcccgcaaggggtagtcatgtgtttttatggagtcctaggtttgattttaggaacctatatttggttaattattctttggaatctgggtgaaggttttaatgaatttaatcgtcaaacaggatatgtgcgaatttttcgttggggattccctggaaaaaatcgacgcattgatcttcaatatccaattcaagaagttcaaagtattcgagttgaaattcaagaaggaattaatccaaaacgcataatatatttaaaattacgtggaaatcgagaaattccattaacacgagcagggcagcctttaagtattcaacaaatcgaaactcaagctgctgaacttgctaaatttttacaagtttctttagagggcatctaaatttatttgctcagtctacttctcaaaaatttcgtttttgggaaagaatagtttctcggcccaaaataaaatattttgggctagaaacggggcagcaaaaccgaaaggaaaaaaaagctgtttgctccaactcattttctacaatttaagaattttctttgtttttgttatttcattttttgaaaatatacaaatcaattggcaattacgcttctaatgttttgtatatttgtataatagaattcattttaaaaaaacttatctatttctttttgattttatctcattttgttaaaaattataaaaagcttacctaaaaaaaagacttttttttaggataacgatgaaggcatttttattcagcaaatctattttttggataaagtaaatgaaagaaaaagtttccttttattattaagatggagtttctttgtcccaaaacagttgtttttggtataagattgaaaggagagatttttattttatgacaaaacgaccacttgaacaaactggattaatcccgcgatcaattctaagaacttttgaacgttttcgaaaacaattacttccaggtgccgaaatgcttgttattcaagaatttcgaatttcaagatatcaagttattgtttctgtaagatgtttaattaccttaatttttgtccctttactagttaacaacctttctaaatcatttttaattaaacctggtgttgaatttctttggaatcaaagtcataatgaaatttttttaaactcatatcaacaaaatagagcattagccgatttacacaaatttgaagagaaactttactttgaatcttttgtagatcctacaagttttaatcaaccagttaatttttctaaagaagttcaaaaacatacggttgaaattgcaacaaaatataatctggaaagcattgaagcgattagtaatctttttgctgattttttaagttttttcagtttaagtcttgtttttgttattttaaagccgcaaattattattttaaaggcttttttatctgaatctttatatagtttaagcgatactacaaaatcttttttattaattttgggtacagatttgcttgtagggtttcactctcctcgtgggtgggaagtttttttagaatggttattaagacattttggactcccggaaaatggagaatttatgtctttatttgtggctacttttccagtttttttagatactgtttttaaatattggatttttcgatctttaaacaaaatttcaccatctactgtggcgacatatcataatatgattgaataatcattaaacttttaaaaactttttataactataaaaaaatattaacgctttgtagtatgttctttaaaaagacttagtatcagtcagcttaccctaaaaaaagtctttttttaggtaagcttttctggaaatcaaaaaaagtctttcttactttctcagctctaaaactaaaaattcaacctttttttagtttcattttagtttcaataagtcccagtacctggtcaaaacaccaagtaaggtttttccttccagtcccaaaaacatagtttttggaactggggggaagaatcttcccaaaaatgacatttttgggaagaatcttcccaaaaatgtcatttttgagacggactgaaaagggctaagattgttgaattttcttaccaaaacaatagttttggttatgaattgtctatagaaactattaacaaaaaacgtacttatgatgctagacttggtgaaatatccagtgattcgtacggaaaaatctactcgcttagttgaaaataatcaattaagttttgatgtagacgtaagactcacaaaaccgcaaattcgaaagctcattgaagagttttttaatgtcaaagtgctggcagtaaacacacacagacctccacgaaaaacaaatagactcggttcaaaacctagttataaacgagcaattgttacagtagattctgacgtgactttattgaaataacacacattgaacttaacctatggctattcgtttttttaaagctgccacaccaggcacacgccacggatctgttttagatttttcagaaattacttcaaaaaaacctgaaaaaagtttgacatcctggtggtctcgttcgaaaggacgaaataatagaggaattattacaagtcgacatcgtggtggtggtcataaacgactctaccgagatattgattttggaagaacaaaagtcaacatccccgcaaaagtaactgctattgaatacgatcctaatagaaattcacgaattgctttagtaagttatcaagatggtgaaaaacgatatattttacacccggttggcttacaaattggtgatactattattgcttcaccagaagctccaatttctgttggaaattgtctaccattggtaaatattccattaggaacagaagttcataatgttgaattacatcctggatcaggaggacaattagtacgagctgctggaactgtagcccaaattgttgcaaaagaaggaaattgggtgactcttcgtcttccttcgggagaagttcgtttagtatcacaaaattgttgggcgactattggtcgtgttggcaatattgaagcttttaatttaactttaggaaaagcaggtcgcaaacgttggcttggtcgtcgtccacatgttcgtggttcggcaatgaacccggttgatcacccacatggtggtggtgaaggtcgtgctccaattggacgttctagaccagtgagtccgtggggcaaaccagcattaggtgctaaaacccgcaaacgtcaaaaatttagttctgctcttattattcaaagaagaaaataatcaagactttatgactttcaactcactccaaaaaattcgtttttgggaaaggttgcagaaatatggttcttcccgaaacataatgttttgggaccaaattgttaatttcagacaaagtcttccccaaaatgccatttttgggataagttttgctgtcctaaatttttcaaatgatccgctccttccctaaatttttcaaatttagggtaaaaactttgtttttgggaaaaggggtaggagtagtaaaaggttaaccttttttcctaaaaaggaatagagagaattatggcaagatcattaaaaaaagccccttttgtggcagatcatttacttcaaaaagttgaacgattaaatacacaaggtgataaaaaagttattaaaacttggtcacgtgcctcaacaattgttccagttatgattggacacaccattgctgttcataatggtcgtgaacatattccagttttcatcacggatcaaatggtagggcataaattaggtgaatttgctccaactcgaacatttcgcgggcatgtgaaaaaagataaaaaatcaaaacgataaacagcttatgggacaaaaagtacatccaatcggatttcgtctcggaatcactcacaaacatcaaagttattggtgcacaactccacaaaaaaatgctttatggattcaagatgcgagttttttaagacagtacctttcaaaaaaatttgttggtgctggaatcacaacgatcgatattgaacggagagatctcgaactccctaccttatctattgaggttcatgctgcacgaccagttctttttttaggtcgcgacaaaaaaggactcgagattttaagagatgaacttgtaaatcaattacgagctttttacagaaaaagaaatttaacagatcctggccgtattcttttaagtctatccgttaaaccactttcttccccagatgctcatgctgccgtgcttgctgaaaaacttgtagaagatttagagaaacgaacaccttttcgaagagcaatgcgccaaactgttcaaagagcattacgtgctggtgtcaagggcatcaaagttcaagtttctggtcgattaaatggtgctgatattgcacgttctgaagatgttcgtgaaggccctgtacctttgcaaacactgcgagcagatatcgattattcttctaaatcagcaaaaaccatttatgggttattaggcgtgaaaatttgggtgttccgcggtgaacgtttaacacgagtaagtaacttttcaacaagtcaaattactaagtaaagaaatcgaaatctatgttaagtccaaaacgaactaaatatcgtaaacatcatcgtggccgtatgcgaggaaaagcttctcgcggcaatacattggtctttggtgattatgcattacaaacattagaggcctcttggattacgtctcgtcaaattgaagctgctcgtcgtgctatgacaagacaagttcgaagaggtggtcaaatttggattcgtttgtttccggataaacctgtaacaatgcgtccagcagaaacccgaatgggttctggtaaaggagcgcctgagttttgggtagcagttgtgaaacccggtaaaattctttatgaattaaaaggagttcctgaaagtgtagctcgagtagctcttcgcttagctgcttcaaaattaccagtaaaaacacaaattcttgtgaaataaaaaactttagcctctttcttatcccaaaaatgacatttttgggacccccttcccccaatttctgaattggggaagggaaggttcgactttaaaagagaaatctggaagaggcccttttgtgtttcttctcacaaacagaatttgtgaggcagggactcaaccctgtttgtaaaaaaggaaaggataaaagagtgttatgattcaaccacaaacgtaccttcaagttgcagataacacaggtgcccgtgaacttatgtgtattcgggttttaggcggtggcaaacaaagagcagctactgtcggtgatattatcattgctgttgtaaaacaagctactccaaatatgcctgtgaaaaaatctgatattgttcgtgctgttattgttcgtacacgaaaaaatgttcgcagaagtaatggaacaagtattcgatttgacgacaatgcagcagtcattataaataaagaaaacaatcctcgtggtactagagtttttggtcctgtagcgcgtgaattgcgtgatggaaattttacaaaaattatttcgctcgcgcctgaagtgttataaattcttcacctaaattaatcccaaaaaaatatttttgggaactgagtggttattttcaatttgcgatagtcacctcttttttcaaaaagaggaaatattcggaaaacaatccaactactccttggcccaaaaactaaattagcccctgcctgaaaaactttgtttttcaggcagggaaaggggacttcccaaaaatgaaatttttgggaaagataaaatattgtttttaggaagggaaagttttctgaattagaaatcgtttttcattccagaatgaaatatttttgggaatgaaatcaaaggagacttttacatatggtgcaacgcttagaaaatttttatcaacagcatagtgtacccaagttgatagaacaatttcattataaaaataaacaccaagttccaaaacttcaaaaaattgttatcaatagaggtcttggtgatgcttcacaaaatgctaaacttttagagtcctgctcaaaagaactcagtataattactggacagcaaggagttgtaacacgttcaaaaaaagctattgcaagttttaaacttcgacaaaaaatgccagttggtgttgttgtcacgttacggggtgaaaaaatgtatgcttttttagatcgtttgatcaatcttgctcttccacgtattcgtgattttcaaggtgtcagtgcaaaaagttttgacggtcatggtaattacagtcttggtcttgaagaacagttaatgttcccagaaattgattatgataaaattgatcaaattcgtggtatggacgtttctattgttacaacagcaccaactgatcaagaagctatggctttattaaaaacatttggaatgccttttaaaaattaactcagactttttcctgtttccaaaaccatgccagtttcctgttcattcccgctccatacccttaatttaataacttcttttttctgaaaaactttgtttcacctaaaatttgatcccaaaaacagtgtttttgggaatgaaaggaagcaagttttttgaaaaaaaaaacaaaacaggactaattcaaaaggcagttttaacaaacatattcaaatagcgttttgtgaaaccatttgcttttaagtaattgaaaaaaggaatcaaaaattattccaagtctgaaaacagaatacagagtttgaaaccatggtaaacgatacaatcagcgacatgttgacacgaattcgaaatgctaatttggcatataaaaccagcgtgtcactctcaaaaaccaaagttaacgagaaactctgtgagattctcgaacaagaaggttttatcgaaaaattttctatttccgaaacacactcaaatgaaattattgttgatttaaaatatcatacacacaagtcacgaggtggtgttggtatcggaaaaccttgtattactaatttaaaacgaattagtaaacctggccttcgaatttatacaaattcacgagaaattccaaaagttctcggagggatgggtattctaattctttcaacatcaaaaggattgatgactgaccgtcaagccagacaagctcgtcttggtggagaaatcctctgttctgtttggtaacgggaagtattcattgtgcccattcccataaacttcaattttgtcacaaatctttttccattgctaaaaacggcgttagtcctttccaaaaatgagatttttgggatgtcgatgtcccttccccaaatttgaaaatttggggtaaaaggaagttttaatcccaaaaacaacgggtttggtaccggattaaacactgttgttaggaaagagacaaacagtccttgtctcaaaaacactgtttatcccaaaagtgtcatttttgggaagggggctggataaaattgctaagttttggaaagggaacgtccttctactgttattcaaatattacatggctcgaaaaaacgttgatcttattgagatggaaggcgtcgtgactcaatgtttgtcaaatggcatgtttcgtgtaagacttgaaaatgggtttcttgtcttagctcatgtttctggcaaaatacgacgtaattatattcgtatattattaggtgatcgggttgctgttgaattaagcccttacgacttacgtcgtgggcgtataacatatcgacttcgcccttcaaaagataaagataattaaaagtatcactatgaaagttcgcccttcagtaaaaaaaatgtgtgataaatgtcgattgattcgacgtaaaggcactttacgggtaatttgtcaaaatccaaagcataaacaacgtcaaggttaattattcaaaatttttactttctgattttctaaacagtctttaatttgagttctttttccaaccactaaaatggttggaaaaagaactcaaattaatggtcatcgaaaactgacagttttaggttgagaaggaaaacacagactctaagaaattatcaaatggtggcttgactttgttttataacaaagtgattctgagacaatttgaaaatatccgttgcattcccaaaaacttgatttttgggaaaaatttttcatttaatcccaaaaacttgatttttgggaccagataaagcaccctttcgttttgtttaaaacagggctggtttgaaaaaaattacattcaaagttttatggcaaaaaaaattgaaaaaaacgcgaaaaagaaatttcgtgaaaaagtcattcaaggtgtagctcatattcaatctacattcaataacactattgttacaattacaactagtaaaggaaatgtcttagcttggtcttctgcaggtgcttgtggctttaaaggtgctcgtaaaaaaacacctttagctgccaagcaagctgctgaaaatgcagcacaaacttgtgtgagtcaaggtatgcgagaaattcgcgtaaatgtcaagggtgctggagcgggacgagaagctgctttgcgtggtctacgcgacgcaggtttaaatatcacaattattcgtgatatcactcctattcctcataatggatgtcgaccacctaaaaaacgtcgtatctaattaaaaatctccaaaagaaggagagatcctgtcccaaaaacttcgtttttgggattaaatgtcagttgctcccaaaaacgaaatttttgggacagggacaccctttgataggtgaaaagaaaccccaaagagtcaaagagtctatgacaaaaactcaaaatttatttttttcttgtgttgagtcgcgcattcaagatcatggctctctgtatggaagatttcatataggtacattttttcgtggtcaagcattaacctttgcgaatgcacttcgacggacattactttctgaaattcctggtgtgagaatcaaagatatttcgattgaaggggttactcatgaatttgcactccttccgggtgttgaagaatcagttttagatattctttttaatttaaaaagtgttgtttttgctccatgttcaacaccaaaaattgaaaattttcgcgtaaaatatgtttcaaaaacagatagtgttggggaagagtttgacatagttcacgaaccagcaataatacgtagtcgtggacctagaaaggtgacagctcgagatattaaacttccgcttacagtcaaatgtgtcaacccagaaacacatattgcaactttaacgggtgatagcgaattcttacttgagttttatttatctctttctaaagatcaatattcttcaacttccccgaaaaaaaatcatactcagcaactttcccaaaaaccagttagacctggaaatgaactttttgagaaaggaagtaaacacatcttaaattttgaaagcgtacctatgccgattcagaaagtaaattatggtattaagtcacttaatgcaaaacaaggatccgaatatattgtttttgaagtgtggactgatggaagtttgcttcctcaagaagcagttgattttggtttaaaacaacttacacatatgttttatcagtttgcttcttttgaaaacaaaacctttgcaaaaaggttcttaccaaaaatttaaggattccattggatataaatcggatggaatttagtcccaactttttgaaattcgcgaagacttcccaaaaacgaagtttttgggatcagattaaaccatgttttttggaaaggaaaaacaaaaacaaagaaataatttactaatttgtcgacagtaaaattatgaaacttatatctacatctcaatcaagtcattcaggtggccgaaaaacagcaatcgctcaggtacagcttattcctggatctggaggtacaattgttgtaaacggaaaaacagccgctgaatactttcaaaacaatgcagtctatttacaaaatttacttacagctactgatcttgtaaaaacggaagcaaaatatgatgctcatgtcgttgttcaaggtggtggactaagtgctcaagctcaagccgttcgattagctttgagtaaagcttttgttgaaatgtttccagactatcgaaaagcttttaaaaaaccgggcttgttaacacgcgacgctcgtattaaagagcgtcgcaaatatggattgaaaaaagctcgaaaggctccacaattctcaaaacgttaaggtttttaaaaactggtagttactggttccaaaaatttttcattattttctcccctgtccccaaatgtcatttggggacaggggagaaaataatgaaaaatattgactatcttagtttttatcactaaagttatcagtattagtgaaaaattttggtaatcccaataaacttgatttattgacgaaaatattctttttgtctagtttttttacatatagtaaaattatttaaattttttctttcttatcagatttatacaattctcaaaacaatcaattgctcctttccaaaaatgagatttatcccctttccctttttccaaaatttgatttgcgggataaaaagtttgtgcgaaagagattttccttctgccatttttcaaatttcgagtaaaacgaagtttttagcgcagggagcgtgaaagattttttagctggttttaatttaaaaccatttttttatatatttagaataaaaattcaatcaaattttaaaaaggagaagactatttatgtctacaacaacaaatgaaattattgaaaaattaaagtcacttactttattagaagctgcagaattagtatcccaaattgaagaaacttttggtgttgatgcttcagctcctgttggtggtggttttattgctgcacctggggcagtaggtggtgcagcagaagctgttgaagaaaaaacaacatttgatgtaattattgaagacgttccagcggataaacgtgttcctgttctaaaagttgttcgcaatttaacatctcttgacttaaaagaggcaaaagaagcaattacaactttaccaaaagctattcagcaaggaatttcaaaagatgatgctgaagcagctaaaaaacaattagaagaagctggtgcaaaagtaaaaatttcgtaaatgcttatccctgccccaaaaaatttagttttggccagggaataatttttctattttttctttaattccttttatcttcatttttatagttgcaaaaaaaatattgtttccaaaatttcttttttccaaaaatttttcaaattttatctcttccccatttactaagtaaatggggaaaaacattgtttttcgaaattgaagggcttccctaattttcaatttttttgacatttttctctttttgagctcagtaggaatcgaacctacattgcacgcttagaaggcgcgtgtcttatccgttagacgatgagccctttcagatctttttaagtttgggttcttatactgttctaaaagtaaaaaaaaccttcaaaaatttaatccggctccttttccattttatagaaaatggagataaaggaaatttttagaattaaaacattatttggaacaaaactcgcatttttgggatatagaacagtggaaattcaaaaaataactttttaaaacaagcgtctttttaatgaaaaaatttgctaatatcagacccaaaaaaatttactttttgggtctgatgtaactagagagtgaaaattacctaaggaggagttaaaaactccttctttccaaaacttcgtttttgaaacaggcaaaactaaaaataaaaattaagcatatccaaaaactcgtttaaaaatatcacgcactttatctccagaatcaattgattctaaaggatcttttcttaatctatggcgtaaacagagaggaataacacggaaaatgtcttctggagttacctcagctcgaccctcaaatgctgcaattgctttgcttgcacggtttgttactaaatcaccacgtaacccatcaacatcaagctctgaacaaatttgagaaatttttactcgataatcataatcaagttgaacttttgaaagaagtttgcgagcttcaatgatttgatttgctaattgtgtttgtgattcttggtatgtttgacgaaattctagtggagctgcgtcaaaacttgcacgttgttcaacgatttgaacacgaaggttagggtctttaactgttccgatttgagcatgcataccaaaacgatctaataattgtggtcgaagttcaccttcttctgggtttcctgatcctacaagaataaaccgtgcagggtgactaatagaaatcccttcacgctctaccgtgttccatcctgaagcagcagaatctaaaagtacatcgacaaggtgatcatcaagaagatttacttcatcaacgtaaagaataccgcgattagcttttgctaaaaggcctggttcaaaagctttaacaccctctgttaaagctttttcgatatcaatcgttccgcaaacacgatcttcagttgctcctaatggtaaatctaccatagaaattttttttgttgtcacaggtaatgtctcattacgttgaagtctttcgcgaacttcttgactcattaattcaggatcttgaggatcagagttaaaaggatcatcagcaacaacttgaatttctggtaaaagatcaacgagagctcgaacagtggttgattttccagttcctcgatctcccataatcataacaccaccaattttcgggtcaattacatttaaaattaatgctaatttcatttcttcttgacccacaatggctgtaaatggaaaaacaggtcgtgcttgttctaatgaattttcaagaattgtattcataaaaattggttttggtttaggtatgtgattctatctttttccgagaattcgcttttttaagtaatagtttttctattttaaaaacaatgtttaatatctgttgtttccctttttctaaggactgcgtttacctcaaatttttcagagttggaaaagaggtagacaaataaactgctagaaaaaacttttctgctagatctcattgtgccatcatgcttggtctttttactgaaaacaaagttttaatcttaatcccaaaaacgaaatggttggaatatcctaaaaaatttatttttttcagataaactaaagaaatctttttagagtgcagcgcatgttatcccaattatgtcacaatttaaaagagaacagattaatttttttacgaaaatctctggtttttaagactgaggcccaaatatcccgaaaatttttatttttgggacatatcggagcgtttattgaagattctctcattgacttttagtatatcagattttttacagcaaatcaattcgtaccagttcaccaaacttgagtcttcaggtataatagattaggaaaaaatgaaaaaaatttttcatttttactctgaaacttaacaaaggttctttaaaatcttctttttgagatattttcttttgcaaagtttgtgaaaagatttttctcaaattgaagttgaacatttttagttacccggtccatttcaaaatgtttcaattgaattaaaaaaacatatttttttataccaaaacttatcccaaaaataatatttttgggaaggatctttgtccagctcagaaaattccttcaaaaatatttgaaaaaggaacccaagttttggaattaaatctataacacttgaacttttagaaatgaaaagaaaaaaagatagaaataaaatttcggttttagggtcttttctttgttccaaaaacaaagtttttggggctggattaaaccatgtttttggtgacataatctaaggtttcagacagagaagaaaatttcaaaaagcttctttggtaacccaaagaagtgtgtaaaatgacaacaaattttatgtttaatttgaaaagttttgaagtcactgcttgtagtgagaggcttctaggtttctcaacaatgttctcaaaagctcttgttgttgtagcttcaagtttctttattgcttcagcaagtcaggcttatcctatttttgcccaacaaaattacgaaaatccgcgcgaagcgaatggtcgtattgtatgtgctaattgtcatttagcacaaaaacctgttgaaattgaagttcctcaagctgttttaccggatacagtattcgaagctgtggtgaaaatcccatacgataaacaaatcaaacaagttttaggaaatgggaaaaaaggtgatttaaatgttggtgcagttcttattctcccagaaggttttgaattagcaccacctgaccgaattcctgaagaaatgaaagcgaaagtaggtaaactttattttcaaccatatagcgcagaaaagaaaaatatttttgtagtaggtcctgttccaggtaaaaagtacagcgaaatggtgtttcctattttagcaccggatccagcaaaaaataaatcaatttcttatttaaagtatccgatttatgttggtggtaaccgtggtcgaggtcaagtttatccagacggatcaaaaagtaataatacaatctacacagcacctgcggctggcaaaattactgcaattgaacctgtagactcaaagggtggttatgttgtaactatccaaactgcaaatggagatctaatttcagatacacttccaccgggtcctgaagtaattgttaaagttggtgatactgtatcagtagatcaagctttaacaacaaacccaaacgtaggtggttttggtcaaggtgaaacagaaatcgtgttacaaaatccacgtcgtattcaaggtttattagtgttcttcctttttgtccttttagctcaagtgtttttagttcttaagaaaaaacaatttgaaaaagttcaattagcagaaatgaatttttaatgttatttttttttctaaaaactttttaaaacacccctttccagtcacaaaaactttatttttgggataaaaattttcaaatttagggtaaaaaagaaatttttgaatatagctcttttccttttccgaaaaacattgtctaatacggtcccaaaaacgaagtttttgggactagaaaaacagcttagtttaagataaaaggtcaaaaaaatgctttcttttcttgcgttttttttccaaattctagaatttgaaactggataaaaaaggggaaaactttgaaaccggagggttaaaaaatggtaaccgtatttagttatataggtttattagtaagtgccttggtaatcactctcacatgttatcttggtcttttaaagattaaattaatttaaacacaataaaaaaaagtctatggtagaacctcttttatcaggtattgtgcttggtcttgttccagtaacaatcacaggattattcgtaactgcgtacttacagtatcgacgtggtgaccaattaaatatttaattgacacattgccctaacaaaaagttagggcattattcaaatctaaaaatttttagttgttaaaaatagccaaaacaatttgaagaatttattcgtctccttttctactctaggggagaattacgaaaaaaaccctaagttttattattcttaccaaatgaataaatttcgaaaaaagaaaaatttttatattataaatttttaaaatgaaaattttttaattaatgcttttgattcttttttgtttttagtttaaatattttttatttttctgaaaattcacgagaaaaactagtaacttgaattatttagcaccaaaagcctatactatttttgcataaacaaacaaaaacgtgatagatcaagctttctagctctggtcgcagtccttaaacttagtttgtagtcaaacttgttaatttagctttgaatcagaacttttgatttcggtttgatactatcacagatttcactttttggcttcacagaaaaaaaaggaaaaaaactcgattaagattaatcgagtatttcctatttgtttcttctcaaaatatgcttcactttggtttaatcacaaattttaaaaaggtagattaaaaccaaacaaattatcctaaaaatttcatttcatcaattctacctaaaaaatacttttttaggatgtcccaaaattgaaaaatttgggatgattttggaagattttttggaaagagagcgctgaagtttcgaaaataacgtaaaggagagagtcacgctatgactatagcgattggaaaaactcaagaaaaacgcggttggtttgatgttgttgatgactggcttcgtcgtgaccgtttcgttttcgtaggatggtcagggttattattattccctacagcataccttgcactaggtggttggttcacaggaactacattcgtaacatcttggtatactcatggtttagcgtcttcgtatttagaaggttgcaacttcttaacagctgctgtatcaactccagcgaatagtatgggtcattctttattattcctttggggtcctgaagctcaaggcgattttactcgttggtgtcaattaggtggtctttggacatttgttgctcttcacgggtctttttctcttattggtttcatgcttcgtcagtttgaaattgcacgttcagtaaaaattcgtccatataatgctattgctttctcagctccaatttctgtattcgtttctgttttcttaatttatcctttaggacaatcaggttggttttttgcacctagcttcggtgtagctgcaattttccgatttattttattcttccaaggattccacaactggactttaaaccctttccatatgatgggtgtagcaggggtacttggtgctgcattactttgtgcgattcacggtgcaactgtagaaaatacactttttgaagatggtgacggtgcaaacacattccgtgcgtttaacccaacacaggctgaagaaacatattcgatggttactgcaaaccgtttctggtcacaaatttttggtgtagctttttcaaataaacgttggttacacttctttatgttatttgttccagtaactggtctttggatgagtgctattggtgttgtaggtttagctttaaacttacgtgcatatgatttcgtttcacaagaaattcgcgctgcggaagatcctgagtttgaaactttctatacaaaaaatattcttttaaacgagggtattcgtgcttggatggctgctcaagatcagcctcatgaaaaacttgtattccctgaggaggttttaccacgtggaaacgctctttaatggttctttagtagtaggtggacgtgatcaagagtccacaggttttgcttggtgggctggaaacgctcgattaattaacctttcaggtaaattattaggtgcgcatgtcgcacatgctggtttaattgttttctgggctggtgcaatgaacttatttgaagttgcacacttcgtgcctgaaaaaccaatgtatgaacaaggtcttatcttattaccacatcttgcgacattaggttacggtgtaggcccaggtggtgaagttatcgatacttacccatactttgtaagtggtgttcttcacttaatttcttctgctgtcttaggctttggtggtgtatatcattcattagtaggtcctgaaactttagaagagtctttccctttcttcggttatgtttggaaagataaaaacaaaatgacaacaattttaggtatccaccttatcgtattaggattaggtgcatggcttttagtttggaaagcgatgtactttggtggtatctatgatacatgggcgccaggtggtggcgacgttcgtatcattacaaacccaacagtaagtcctggtgttatctttggttatcttttccgttctccttttggcggtgacggttggattgtaagtgttgataacatggaagacgttattggcggacatatttggattggtacactttgtatttttggcggaatttggcacattttaacaaaaccatgggcttgggctcgtcgagcttttgtttggtcaggtgaagcttacctttcatacagtcttggtgctatttcaattatgggtttcactgcatgttgtatggcatggtttaacacaacagcttatccaagtgaattctacggtcctacaggtcctgaagcatcacaatcgcaaacatttactttcttagttcgagatcaacgtttaggagctaatgttgcttctgcacaaggtccaactggtttaggtaaatacttaatgcgttcaccaacaggtgagattatctttggtggtgaaactatgcgtttctgggatttccgtggtccttggttagagcctttacgtggccctaacggtttagatttaaataaactaaaaaatgatattcagccatggcaagaacgtcgtgcggcagaatacatgacacacgctccattaggctcattaaattcagtaggtggtgtagcaactgaaattaacgcagttaactatgtttctccacgtagttggttagcaacatcacatttctgtctaggcttcttcttctttgtaggccatttatggcatgcgggccgtgctcgtgctgctgcagctggttttgaaaaagggatcgatcgtgataatgaacctgtattatcaatgcgtcctctagactaaaatttttcctaaaaaatttttttaggatttattccacccccaaaaacagtttttttgggatggacctttttctaaatgtttcactttttatcaggtgatatcaataaaaaggaaatgctggtgtagacggaaatatttccgtctacaccaatacttaatttgaaagattaacaacattaacacttatcactaaatacatttcacaaacaaaacttcaacaaaaaagaagagacctccttcctcacaaattttgtttttggggccagatacttgacctttttgttttttctaactataatttattctaacctacaaaaatcgaggagagatgaccgagaggccgaaggtagcggtttgctaaatcgccgtatggtttgccataccgagggttcgaatccctctctctccgatgttccgatcttttcttaatttgtatgtcttgcggcttgttcccaaaaactctgtttttgggaaaaaaattttcaattttattgattacaaatgaaagaagtccatatttagtattttcaacaatttttcttttacttttctagatttttgctgtgtaaattgtcttaaagagtttttttattttttttaataaaaattttgccaatccttttggcgatttcttattgcagtataggaaaaattcccaatttttggattggaatgacagcagattaaaaaaatctagattttttgtttgtctgtgatatactaatagagagtagtagcctataaagctttcactttgttttcaaattaagtatgttaacacttaaaatttttgtttatactgtagtaacatttttcgtatctctttttatttttggtttcctttctaacgatccaggacgtaacccaggacaaagagaacttgactaaaaccctcattttcactttccggcgccttgtgcattccttttccaaaaaattgtctttagccccaaaaactttgtttttggggcagagagtaattctgttttttgtataaaattgaaaatttttgggtaaaacggggttccatacttgtccccaaaccttcaaagtttggggtcaaaatttcgtttatccaccaaattttgttatcctcacaaacttgattttttggaaagggggaaagaaatattatcaaatttgaaaaagtgggaaacaaaataacaaatttttttggaaaaaaagaaagagaaaaaaaaaaccatcgatttttaggggagatctcttctatgccacgctcacaacgaaatgataattttattgataaaacttttactgttattgctgacattttattaaaagttttaccaacatcaaataaagaaaaacaagcttttagctattaccgagatggtatgtcagcgcaatcagaaggagaatatgctgaagcattacaaaattattatgaagcgttacgtctcgagacggatgcttacgatcgaagttacattctctataatattggattaattcatacaagtaatggcgatcatgcacgagcattagattattattatcaagctcttgaacgtaatccttcattaccacaggcattaaacaacattgcagtcatttatcattatcgtggagaacaagcgctcgaaagtaaccaaacagaaatttcaaaacttctttttgataaagctgcagattattggcgtgaagctattcgtcttgctccaacaaactacattgaagcgcaaaattggttaaaaatgactggacgattctaataatcgttttattgatctttttcagtttgccttcactttccttccccccaaaactgtgtgtttggtagacgttttttggctttctcaaaaatgtcatttttaggacaagttgagttgtttttctcttaaaattgaaaaagccaactgcatttttatagataaactgataaactgaaaaccctgtttttaaatttcatctctgcttttctaacattgaaaattttgtatacataatatttttttcataacgtaagagaagctatatttgttggaggtgaaggcttgtaactcagtggaattagagtccgtgtttccgacacacggtgtcgtgggttcgaaccccaccaagcctagtgaaattcgaaatagattttttttgggaaattttttctaatctgtttctttacaaaatttttctttttatgttttagaaaaaaatactatttttatatgttgctttttaaaaggtttcccgataaaatgaaattatcgaaaacctaaagcctgcttaactcaatcggcagagtatcggtcttgtaaaccgaaggttatcggttcgactccgatagcaggctattttagacctttcgcttgtaaagctttgtttttttggtcataatgactaatgaattacaaaaacgaaaaattttttgtcttttttattcttttttcctttcacaaaaatataatttagtcccaaaaaagaagtttatccccaaatttttcaaatttggggacgaggtaggggttaattaagtttttccaaaatttgaaaaatttgggaaatgggaagggaatatttgaagaggttcaataaagttgattttcaacaaagatcaaaagtcttcgtttttatttatacatgattgatagaacttgacaaaaatttattttttctttatttgtcaactaaaaaaatatcaaaaaatgacatgcttcaacgagtaaaattagaagacatgattcaaagtgggatgcattttggtcattccactcgtgaatggaatcctcgaatggcaccttatatttatggagaacgtaacggtcgccatgttttagatttagtacaaacctattatttgtttcaaaaggttggagaatttcttgaagagtctgctgcccaaggaaaaacatttctttttgttgggacaaaacaacaagctgcaccgttaattgctaaaacagcattagggtgtaatagttattatgttaaccaacgttggcttggtggaatgttaactaattggcgcactattcaaaaatcacttcgcaaacttcaagattatcgtcaagcagaggaacgtggcgattggaatctcctaaaaaaacaagaagtagctcgtaaaagacgtgaaaaagatcgtttagaaaaatatttgtctggtgttgaaacaatgtctcgtttacctggagttgttattattgtcggtcaaaaagaagaaattcatgctgttaaggaatgtcgacaattaggtattccaacagtaacgcttttggatagcaattgtgacccaagtttggcagattggtttcttccagcaaatgatgattcagtttctgcattacgtttagttctaaataaatttcaagaagctattttgtcgggtcaagctcgttttgttgagcgtgaagcatcacagaaaacaaaacaaaaaacaaaacaaagacaaaaaacgaaacaatttcgtggtcgtcgatcttcttcaacaccaagaaaaagaaaagcggaaactccaaaactttaattttgtttttcttacctaaaaaaggtattttttatcccaaaaactttgtttttgggactggatgtccttttcttttccacaaaaactgccttttacgccaaatttcaaaaatttatctcttccctgtcccaaaaatttcatttttgggattaaatcaaatttttgggaacccctttcccatgtactttatttatttcacatttgagaaattttgggaatggaagtcgcataaactaaagggaccaaaggttctaaactttgtaaatctcaatttttctaattcgagaatgtaaaaggacaggaaaaaactgtttttattaaaaaacagaaaaataactatcacttagtttgtcccaaaagtttgcatttaaacccaaaaacgaagtttttggggtagggaccaaacaagactacaaaagaggatagagtatatgacaaacatctggtttgatgttgcagaagtttctgtaggccaacattggtattggcaagttggtggctattcagtacatggccaagtattaattacatcgtggattgtattagcagtgattgcagttattggtcttttaggagctccaaaacctcaacctgtttctggtggatcatcaacacctgaaggtctccaaaatattacagaatatgtaactgaatttatccgtgatttagccaaaactcaaattggtgaagaagagtacttaaaatgggttcctttcttaggaacaatttttctatttatttttgtttctaactggtctggggctcttttaccttggcgtattttagaattaccaaatggagaattagcagctccaacaaatgatattaatactacagtagctttagctcttttaacatcaattgcatatttttatgcaggtattagtaaaaaaggatttggctattttaaacgttatatttcaccagctgcttttttattaccgattaacattcttgaagattttacaaaaccattatctttaagtttccgactttttggaaatatcttagctgatgaattagttgtaggtgtattaattactttagtaccacttgtaattccaattccaattatgcttttaggtctgtttacaagcgcaatccaggctcttgtttttgctacattagcaggcgcttatattggcgaatctgttgaagatcatcattaatttttttgtttaggaattctttaaatttactttttattcctttccattctattcccttttccaaaaatttcatttttgggattgaggaagggataaatttgtcaaagttgatgctgcctaaaacgctcatttttcgtttaagaaaggagaatttgttcaaataaaagaagtccaatctcttttcaatcgtgaaagaataattttcttatattttttactttttatgattagaacttctttattttatttaaaagggtttgttaagtaaaaagtgctggttcggtaattgaaaattaccgaaccaagatattattaaaaaagaaaattctgttttttcacaaaacacgttctacttgaaatttcaaaaatttaggaaaggaaaatcaaaaggaaaactgattctaagcaattaaaaaaagatttgcttttaattttttgagttaaaaaatattagtaaatttttcagcgcattttttaattaaaaaaaactgtttgaaattaaaaaatgcgctgaactaaacgtttttttaatttcatcttaagaaagtttttttgatgtttgaaaaaattgcatttgtaagcctttaagtttttttttcctgactttaggaaatcagaaaaatcagctttgttgcacttgaaaacttcgagaatacacattatcattgaaatgacaccaggagttcttccaaaaaattagatttttggaaagtgattgcaccaatgaaggtctcattcggtccctaaactttcggttttaggatcttcctgcgctcgttccagttcatttcccaaaaaaagacttttttggggatattgtgattcgattttgacatatcgatcactaacttagtaacttttttacatacacaatccattggaggttaaagaattatgaatccacttatcgctgctgcaagtgttgttgctgctggtcttgctgtaggtcttgctgctattgggcctggtatgggacaaggaactgctgctggttatgcagtagaaggtattgctcgtcaaccagaagcagaaggtaaaatccgtggtgctcttttattaagtttcgcattcatggaatctttaactatttatggtcttgttgttgctttagctcttttattcgcaaacccatttgcttcttaatttttccaacgccttgatttcgtatccctttctcctttttccaaagccttttgtttaaatcctttcccaaaaaattcatttttgagagtaacgtcgtttctttccttcttaaaatttttcaaatgtagagaagggagaagggcttctcaaaaatggtatttttgggataggtagtgattaaaagcgtttttatgtcaaaattctccatgattttcaaaaactttgtttttgataaaagaaatgaataccatttttttcaaaaaaaaactcttcacaaaacagactgttttgggattagggtaagggttattttaagagcgggttcacaagaattgacaaacaagtagactcatcccaaaaacaaagtttttgggaagggttttactcttcaaattttttttgaagtcttgtgaatttcatcataatattaaggagattttatgcttttcagcacttctttagtcttggcagggcattttggctttaatacaaatattttagaaaccaatgtgctaaatttagccgtggtgctggctatcgtcctaacatatgttggagatgctttacgtggtcttcttgaaaatcgtaaacaaactatcttggcaaattttcgtgaagctgatcaacgtgcaacagaagcacaagatcgattacgccaagctcaacaagaattagagcaagcacaagtaaaagctcagaaaattcgtgagcaggcaagtgtgacgattgaacaagagaaaaaacagtttgttcgtcagacacaagaagacattaaacgtttaggtattcttcaacaagaaaccttaaagttcgagcaacaaaaagcgcaaaatgaacttgctcaaaaattagtcaaattggcattacaacaagttcgtgaaaaactgaatcaaagattaacatcttcaattcatagtgcagtaaataatttccaaattgtactctttacgaattataaagccagttaaaacacttcctaaaaaatctttttaggatgaccatggctcaaaaacctttttaattcttaaccattgttgaccccaagaagtctgtttagacacctttccttttcccataaatttgttttttggggttggatgacactttgtttgtggaaaccaggctccccaaaaatcccatttttggaagggagctgctttcgtttttgggaaagaaaaataaaaagatttgagctgtgtcacaaggaggaggcggttcagcataccatggtaaaaattagacctgatgaaattagtagtattatcaaacagcagattgagcaatatcagcaagaagtcaaagctgtaaacgtagggacagtcttccaagttggggatggaatcgctcgtatttacggtcttgataaagttatggctggtgaactcgttgaatttgaagacggtacagtaggtattgctttaaaccttgaagcaaaaaacgttggtgcggtacttatgggtgaaggaacaactgtgcaagaaggaagttctgtccgtgcaactggtaaaattgctcaaattccagtaggtgatgcgtttttaggtcgcgtcgtgaactctcttgctcgtcctatcgacggcaaaggtgaaatcgtgacaaaagaaactcgccttattgaatcacctgctccagggatcatttctcgtcgttcagttcatgaaccattacaaacaggtattgttgctattgatgcaatgattcctattggccgtggccaacgtgagttaattattggtgaccgtcaaacaggaaaaacagctattgctgtagatacaatcttaaaccaaaaagggaaagatgttatctgcgtttacgtagcaattggacaaaaagcttcttctattgcacaagtagtgaatacacttcaagaacgtggtgcgatggattatacaattattgttgctgcaacagctgattctcctgcaacacttcaatatttagctccatatactggtgctgctttagcagaatactttatgtatacaggtcgtcatacacttgttatttatgacgatttaacaaaacaagctcaagcatatcgtgaaatgtctttattacttcgtcgtccaccaggacgtgaagcctacccaggagatgttttctatttacactcacgccttttagaacgtgcagctaaattaaacgataaattaggtagcggtagtatgactgctttacctgtagttgaaactcaagaaggtgacgtttcagcttacattccaacaaacgttatttcaatcactgacggtcaaattttcttatctgccgatatctttaatgctggtattcgtcctgctattaacgtaggtatttcagtatctcgtgttgggtcagcagcacaaccaaaagctatgaaacaagtagctgggaagttaaaattagaattagctcaattcgctgaattagaagctttttctcaatttgcttcagatttagaccaagcaactcaaaatcaattagctcgtggccaacgtttacgtgagttgttaaagcaaccacaagcttcacctctttctttagaagatcaagtagcaggtatctatgctggtacaaatggttatttagatgttattccagctgatcgtgtccgtgcatttttagttggtttacgtcaatatcttgcaacaaataaatcaaaatatggtgaacttgttcgttctacaaatgctcttacagatgaagctcaagctcttttaaaagaagctttaaaagaatatactgaagagtttttagcaagttctaaataacttttttttcaagtttttactgatttgtttttcccttcctttaaaccgattttcttttttctcaaaatcaatgttttttagtctattcattcccaaaaccatagtttttggaataaagtttttcaaatttttgataaaaaaacttggtttttttcttttccacaaaaacgaaattaagcgatcgtttttgccccaaatttgaaaaatttggggaagtaagagaaaaatacaatgaaaaaaaggagtaaggaagggagaatcaaaggcagcaatttagtcaccagattatttttttttcttagtctcaaattctttttttgcaagaagtttttaaagagttaaacgttcattattagaaaaaaactcaataaattcttgctttttctttttgtataggttactatatttagtataaaaaccagaaatgatagcctgcttagctcagttggttagagcatccgtctcatacgcggagtgtcactagttcgaatctagtagcaggcaccacaatctatttgcgggtatagctcagtggtagagcggcaccttgccaaggtgcatgtcgcgcgttcgatccgcgttacccgctttttattcttgatccgaaaagcaaaaattttaatgtcgctaattacccctttccctttctctaaaaatttctcaaaacagattcaaacatgtttgtgagaagagggaggaaattaaatacaattttttgaaatgaggaaagtgttgttagataaactaaatgtgttcttaattaaaatgatcagggctaattccatactaaactcatattagttgaaataataaaaccaaatgattgcttttttcaagaactttatggtagacttaccctaaaaaactaaaaggggatgtagttcaatcggttagagcaccaccctgtcacggtggaagttgcgggttcgagtcccgtcatcctcgacacagaatttgtctttaatttttattaagcatcttgaatatgaccttagttaatgctatcctaaacctaggcgtaaacgcgctagggaaaactttgttttgcctaggttccttaaaaagacctttattactttctggtaatttcaaaagatttcttttcaattcagaaagttgaataattcgtataactcgaacatcatagtgtgtaaattatttgagaaaataaaaaacaacaatccatcccaaaatttttcaaatttttggaaaaacagtatttaatcccttttccaaaatgtgttttggaaaagggagggaaggttaattttttacttttgaaatgctattttttacaccaaaaagattgattttttagtcttttgatactgacaaccattcaaaaagacaaaattcaatatataagaaaaaaagtatacaaaatatgagcgtttctacagaaacaaaaagtattggtcgtattactcaaattattggtccagtattagacgtatcatttccacccggaaaaatgccaaacatttataacgcattaacagttcgtggcaaaaatgaagctggacaagagatttctgtaacgtgtgaagttcaacaactattaggtgatcattgtgtgcgtgctgtagcaatgagtgcaacagatggtttaatgcgtggtatggaagttgttgatagtgggaaaccgttaaatgttcctgttggtcaagctactttaggtcgtatttttaacgttcttggtgagcctgttgataaccttggtccagtaaataacaaagatcaacttcctattcaccgttcagcaccagcttttgtggacttagacacaaaactctctatttttgaaacagggattaaagttgttgacttattagctccttatcgtcgaggtgggaaaattggtttattcggtggtgcaggtgttggaaaaactgtacttattatggaattaatcaataacattgcaaaagcacatggtggtgtttctgtattcggtggtgttggtgaacgtacacgtgaaggaaatgacctttacatggaaatgaaagaatctggtgttatcaatgaatcaaacattgctgaatcaaaagtagcacttgtatacggtcaaatgaatgaaccaccaggagctcgtatgcgtgttggtttaacagctttaacaatggctgaatatttccgtgatatcaataaacaagacgttctactttttattgataacattttccgttttgttcaagctggttctgaagtatcagctcttttaggtcgtatgccttcagctgtaggatatcaaccaactcttgcaacagaaatgggtggtttacaagaacgtattacatcaacaaaagatggaagtattacatcaattcaagctgtatacgttcctgcggatgacttaacagatcccgctccagctacaacattcgctcacttagatgcaacaacagtactttctcgaaatttagcatcaaaaggtatttatccagcggttgatccattagattcaacttcaacaatgttacaaccgtggattgttggtgatcaacattatcaatgtgctcaaaacgtaaaacaaacacttcaacgttataaagagttacaagacattattgctattcttggtttagatgaattatctgaagaagatcgtcttatcgtagctcgagctcgtaaaattgaacgtttcttatcacaaccattctttgttgctgaagtattcacaggttcacctggaaaatatgtaagtttagcagaaactattcaaggctttaaccttattttatctggtgaacttgatgatcttcctgaacaagctttctatttagttggtaacattgacgaagcagtttctaaagcagcaacgctttcgtaattgatgcactttacctaaaaaaagacttttttaggatgtcccaaaaatttatttttgggactagtggttgaaaccaaaaaaaataatttcttgtttgttgaagacaaccatatttagccacaccacaactatgacattgcaagtttgtatcatgacccctgatcgtatcttttggaatgatcaggcagacgaaattattcttccgactaatacgggtcaaatgggtgttttaacaaatcatgcacctttaattactgcattagacataggtgtcactttaattcgatctaatagtaattggattcctgtagcccttatgggtggttttgcgttagtaaaacaaaatcaagtgactatcttagtcaatgaggcggaatctgctcaaactatcggagtagatgaagcagagaaagcttttgaagaagcaaaaacacgattagaacaatcacaaggcgaaaaacaacgcgttgaagctacttttgtttttaaacgagcacgagctcgttatcaagtagtaaaacagcttgctgtgtaatcctatttttcaaatttggggtaaatctcatttaccccttctctatccttcaaatgaaatttggaggaaagacttcccacctaaaaaaatacttttttttatcccaaaaacgaggtttttgggattaaatgatatttttgggatggggggttaaaccctctttttttaaataaggaagagcaaagagaaaaaacttggaagcttgtaaaaaagattgttctttaaaaattaaaattctcacgattgagttttcattttattaagagagttttcgagtgtattcttttctaaaatttttttagttttaatgaatcttggcgtttctgcttatgttttctctcttaaaaattaaaaagtttgtctggttctttccaaaaatcttagtcactcaaaatttttcaaattgataccctccatttctaaaaacctcgtttttggaaatggaagcggtgggttaaatttttttaaagaaattaaaaataagcaaattaactgtcacttttagacaaaactaaagaaacatttttacaacacaatcgaaaaatttgtaactcaattaaattacagttcggtttaaaacgaatacttctctttcaacttacccgaaatttggaaattttggggaaagaaagaaacaacagagaaaaaaacttgttataaaattatttcatttcaacttattattttatggctcgatatagaggtccaagattacgtattgtccgccgtttaggtgaattaccaggattaacacaaaaaaattgtacacgtgatttcccacccggacagcatggtccaaaaaaacgtggtggcgctcaaaaagcaaaagaatcacaatatgctgtacgtttaaaagaaaaacaaaaattacgatttaactatggaattagcgaacgtcaattaattagttatgtgcgtgaagctcgtcgacgtaaaggttctacaggtgaaattttattacaattattagaaatgcgattagataatatcgtttttcgtttaggttttgctccaacaattgcggcagcgcgtcagcttataagtcacggtcatattttaattaataatcatcgtgtaaatattccaagttacctttgtaaggttaatgattcaatctctgtagcttcaaattctcaaaaatttctaaaaaaatctattgaaacttttacaaatagtctttctgctcctcatttagaacttaatacagagaaattccaagctgttgttcgagatactgtatctcgagaaagtgttggattacaaattaacgaattacttgttgttgagttttattctagaaaagtttaaaaactttttcacagttaaagtccgaacagaaaaagaatttaaattctttttctgtttttagtacctgtttctttcccaaaaatgacatttttgggaccaactttgatttgggatgaaaattcctctagattttactttttttcttgataaatgaaagtcaatcttagtatttttagaaatgttgaaaatatttgaaaagaaaaaaatcttcattcccaaaaacttttcgaactttaatccagacccaaaaacaaagttgttggcattaaagtaatgttttcccgaaaaattttcaattttggggcaagaatttttaattttttatacattaaattaatgtagacaagtttttttaaattgattaaaaattttgaatttttgccgttacccggactcgaaccgggatggacctattgtccgcaaacccctcaagcttgtgtgtctacctatttcaccataacggcttacgttattttagtatatcatatttagatttaagtaaaaaatcaaatttagaagttagagcatcagtttttatataagaaaaaaagggtaggggcttttaaaaaagaaaaatctaaacaacattagtaaatcaaaatcttaagtaaaccaattcaaattacggaaagagagggattcgaaccctcgatgatttttcaatcatatagacgttccagatctacaccttaaaccactcggacatctttcctttttgttctaaaattttttaattgtatcatttcttattcttttcccgatcccaaaaacttttctatcaaaaatttttcaaatgtgtaagtgttttttattgtttttgagaaaggccataaaaacggacaaacgtgtttttataagaacctttaacttctcgaaaaaaaaatttaattacaagagaatacatattagttttttattttctagtatacttcacatgatctcaaaattttggtttatcccttccccaaaaacttcgtttttggtatgaaatttcatttaagtaataaagaaaggggataaacaaagttgggataatccacttaagggttagtctagggtttgaaaggccaagacctttcaatattttttcaattctttttttggtcaaaatcttactattctggtccaattttaatgattacaaaaacgactgactcaaaaccatagtgttaatatcagctgctaataagttcgcttcaaaccaattataatagaccttattttattcgtatgcaatcatatcggctaaattagcggcgtgagctaagtctgtccgattagtaccaaacactgaatagcagagaaaacaggcaacgataaatgctctaaagattggtcaatcggaactaaatccataaaataaagtcattcaaattttattaaaaagtgtgtagtcagtcccaaaacattgtgttttgggaagagtttttcaaataaataaaaaaactctatttctatttctgttaatgcaagtacaagaaagagaggcagactgggttgacattttttggcattcacagtacattagtaactgtgaaaaacaaaaaagttttcacggagagtttgatcctggctcaggatgaacgctggcggcatgcttaacacatgcaagtcgtacgaatgcaatgaggcttgcttcattgtaatgagtggcggacgggtgagtaacacgtaagaacctaccttttggagagggacaaccattggaaacgatggctaatacctcgtattgctgagaagtgaaagatgaaaatcgccaagagatgggcttgcggctgattagcttgttggtgaggtaatggcttaccaaggcaatgatcagtagctggtctgagaggatgatcagccacactgggactgagacacggcccagactcctacgggaggcagcagtgaggaattttccgcaatgggcgaaagcctgacggagcaatgccgcgtgaaggatgaaggcctatgggttgtaaacttcttttctcagagaagaattttgacggtatctgaggaataagcatcggctaactctgtgccagcagccgcggtaagacagaggatgcaagcgttatccggaatgattgggcgtaaagcgtctgtaggtggcttcaaaagtctcctgtcaaagatcagggcttaaccctgggccggcaggagaaactctgaggctagagtttggtaggggcagagggaattcccggtggagcggtgaaatgcgtagagatcgggaggaacaccaaaggcgaaagcactctgctgggccataactgacactgagagacgaaagcgaggggagcaaaagggattagatacccctgtagtcctcgccgtaaacgatggatactagatgttggataggttaaagcattcagtatcggagctaacgcgtgaagtatcccgcctggggagtatgctcgcaagagtgaaactcaaaggaattgacgggggcccgcacaagcggtggagcatgtggtttaattcgatgcaacgcgaagaaccttaccaggacttgacatgccactttttccctgaaaggggaagttccagagtggacacaggtggtgcatggctgtcgtcagctcgtgtcttgagatgttgggttaagtcccgcaacgagcgcaacccttgttttgaattgccagttatgggaaattcaaaagactgccggtgacaagccggaggaaggtgaggatgacgtcaagtcagcatgccccttacgtcctgggcgacacacgtgctacaatggccgggacaaagagatgcaaacccgcgagggctagccaacctcaaaaacccggtctcagttcggattgcaggctgcaactcgcctgcatgaagtcggaatcgctagtaatcgcaggtcagccatactgcggtgaatacgttcccgggccttgtacacaccgcccgtcacaccatgggagctggctatgcccaaagtcgttactccaacccttgtggagggggacgcctaaggcagggctagtgactggggtgaagtcgtaacaaggtagccgtactggaaggtgcggctggaacacctcctttaaaaggaaataaaaaaataaagagttcttttcccgaaaattttattttcgggaaaagatcaggtcacacttttttgtctgaaattttcaaattttgggaaagaatgtgcatgagtcactaccttacttttagaattgctgagtttcaaaaatttcctatgaaaaagaaaggaaaagactcaaaacttttgggaaaaggcaaagcaacgggctattagctcagttggttagagcgcacccctgataagggtgaggtcgctggttcaaatccagcatagcccaccacggtgctatccatattcttcccaaaacatttaatttttggaaaggggatatagctcagttggtagagcgctgcttttgcacggcagatgtcagcggttcgagtccgcttatctccaccagttatctaaatataaaaagtaaggtagtggctcatgcacacgcacatgcgattctgttctcaaaaacaaaaatgtgtgacgtcctaaaaaaattttttagggcaaaatctcatgaaccaaaaaacaacaaggtcaaatgaagtaaggcgtagggtggatacctaggcacctagagacgatgaagggcgtggaaccgacgaaacgcttcggggagttggaaacaaacattgatccggagattcccgaatagggcaacctcatgtacttctacctgaattcatagggtagaaagaggcaactcagtgaactgaaacatcttagtagctgaaggaaaagaaagcaaacgcgattccctgagtagtggcgagcgaaatgggaacagcctaaaccaagtgtttcacttggggtcgtgggaaaacaaaaaaaaacacagttgatcccaaaaacaaagtttttgagagacatttaaacgaagcagctgaaacctgcaccatagatggtgaaagtccagtagttgaaaaatgaaactaagtttgtgtttatcccgagtagcatgggacacgtgaaatcccgtgtgaatcagcgaggaccacctcgtaaggctaaatactcctaggtgaccgatagcgaaatagtaccgtgagggaaaggtgaaaagaacccctgtaggggagtgaaatagaacatgaaaccctatgctgacaaccagtgggaggcatctaaatgctgaccgcgtgcctgttgaagaatgagccggcgacttagaaaacgtggcaaggttaaggaaatatatccggagccggagcgaaagcgagtctgaacagggcgattaagtcattttttctagacccgaacccgggtgatctaaccatgaccaggatgaaacttgggtgacaccaagtgaaggtccgaaccgaccgatgttgaaaaatcggcggatgagttgtggttagcggtgaaataccagtcgaactcggagctagctggttctccccgaaatgcgttgaggcgcagcggttcataaggctgtctaggggtaaagcactgtttcggtgcgggctgcgaaagcggtaccaaatcgtggcaaactctgaatactagatatgctatttatgggccagtgagacagtgggggataagcttcattgtcgagagggaaacagcccagatcactagctaaggccccaaaatgatcgttaagtgacaaaggaggtgagaatgctgaaacaaccaggaggtttgcttagaagcagccaccctttaaagagtgcgtaatagctcactggtggagcgttcttgcgccgaaaatgtccgggactaaatgatctgccgaagctgtgggatatttctaaaatatcggtaggggagcgttctgctgtagggtgaagcaatgatgtaagtcattgtggacgaagcggaagtgagaatgtcggcttgagtaacgcaaacattggtgagaatccaatgccccgaaaacctaaggattcctccactaggttcgtccatggagggtgagtcaggacctaaggcaaggccgaaaggcgtagtcgatggcaaacaggttaatattcctgtactatttgatatttggtaccgagggacggagcaggcagagattggccgagaatggatttcggtggaaactttcgacacggtgagaggtagaagaaaaactaaccttgagtgaagaagtgatacgtctttatctttggatagagatcagttgactgtcatactcccaagaaaagctcgaactactgtaaaatatcaaatacctgtaccgtaaaccgacacaggtgggttagtagagtatactaaggggcgcgagataactctctctaaggaactcggcaaaatgaccccgtaacttcgggagaaggggtgccaagtaactcaaattgcttggtcgcagtgaccaggccctggcgactgtttatcaaaaacacaggtctccgcaaagtcgtaagacaacatatgggggctgacgcctgcccagtgccggaaggttaaagaagttggttattccttaaggaagaagctgacgactgaagccccggtgaacggcggccgtaactataacggtcctaaggtagcgaaattccttgtcgggtaagttccgacccgcacgaaaggcgtaacgatcagggcactgtctcggagagagactcggtgaaatagacatgtctgtgaagatgcggactacctgcacctggacagaaagaccctatgaagcttgactgtagcttggaattgggttcgggctcttcttgcgcagcctaggtgggaggcgttgaaggttttctttcggggaaactggagccaacagtgagagaccactctggaggagctagaattctaatggcgttccttgaagcaggacgcttgacagtttcaggtgggcagtttgactggggcggtcacctcctaaaaggtaacggaggtgtgcaaaggttccctcaggctggacggaaatcagccggagagtgtaaaggcagaagggagcttgactgcaagacctacaagtcgagcaggggcgaaagccggccttagtgatccgacggttccatgtggaagggccgtcgctcaacggataaaagttactctagggataacaggctgatcttccccaagagttcacatcgacgggaaggtttggcacctcgatgtcggctcatcgcaacctggggcggtagtacgtcccaagggttgggctgttcgcccatgaaagcggtacgtgagctgggttcagaacgtcgtgagacagttcggtccatatccggtgtaggcgttagagcattgagaggagccttccatagtacgagaggacctggaaggacgcacctctggtataccagttatcgtgccaacggtaaacgctgggtagccatgtgtggagtggataaccgctgaaagcatctaagtgggaagcccacctcaagatgagtgctctccattaggtcacaggaagacaacctgttcgataggcagaaggtgtaaggccagtaatggtttcagctgatctgtactaatagaccgaatgattttgaccatacacgctttctctcttttacatgtttttaaaacatgtaaaagagagaaagcattaccttgtattcttgcttgcatggaaccaccttatccatcccgaactaagaagtgaaacgtgcaaggggcgacaatacttgaggggaggccctctgggaaaatagtctaatgcaaggtaattgtaaatacaaatatttatacaaatgttttatcggagtaaaaaacttaacagtctatacagtctatttttcatatttttacaataaatagcttaaaaaatataaaataaaactttcaaaatttatccttgctaaagaggacctcgtaaagtaactgtttgttttaaaatccctatacgatcaatagcccctaaagcaaaaacataaataatgttatgggtacggtatctttttgatcccagtattcaaaaccttttactagattgactttattgagttttttatttatattgaatataagcctttgtgacggaatggtagacgtgccagttttaggaactggtgtcttttgacgtgtcggttcgagtccgaccaaaggcactgtgacccttgtccaaaattagttaacatggaccagaaaacttagggttttgaaaagcttgttttttgttgttttctcttttatactttatttaaaacaaaggcctttagctcagctggtagagcggctgccttacaagcagcgggtcatcggttcgaatccggtagggcctaatctaaatttaatcttctctttctatttccccataattcttatggtacgagaaaatctcttagagtgaggttgttgactgttttttctcttttacttttttttattttttgaaaaattttgttttttgaacagaaatgatttatgaggggttaatcaacctgctctcaaaaacgaaatttttgagagcaggttttattattcttttgtttttgattgactattctaaaaccatcgtttttgttttcagagtccgatataataaaagaaagaatatcagttactgacaaaacttaagttttgtcaaaaaggtgagtcaaaacttcgtttttagacttcctttttcctttcccaaaactcagattattatcacttccaatcttttcaatttttatcagaaatataaaattgaaatcttaaggaaaagataactagaatcaaaaaatttgtttttggtgcatattttggctaaaaaatcaaaggaatttatatgagtgctcaatttttacctagcattttagtccctttagtagggcttgtatttcctgcagtagcaatggcttcaatgtttttatacattgaaaaagaagaaatcaactaaaagtttttttattcagttagccttttttgtgtctagttttactttgacacaaaaaaggcttcccaaaaatttttccgaagattcatttatgaagaatttttttagaaggaaaaaattctatcaaaaaatatcaaaaaaatccaactcacacttaccctttgacaggagaatttagttttggtatactgttaatatcttaaaaaaatttttagataaaggaggatcttttatgtcaattctttcttggcttgaaaatcaacgaaaattaaaactattaaatgcacccaactacagtcagcaagaatctgacggaagtcagggtctctggacaagatgtgatcattgtggtgtgattttatatattaaacatttaaaagaaaatcaaagagtgtgctttggttgtgggtatcatcttcaaatgagtagtacggaacgtatcgattatttaatcgatacaggaacttggcgtccttttgatgaaacagtttctccttgtgatccacttgaatttaaagatcaaaaaccgtatacagaaagattaaaagacgcacaagaacgaacaggattacaagacgctgttcaaacaggaactggtcttcttgatggcattccggtagcattaggtgttatggattttactttcatgggtggaagtatgggctcagtagttggcgaaaaaattactagattaattgaatatgccactcaagaaggcttacctgttattttagtttgtgcttctggtggcgctcgaatgcaagaaggtattttaagtttaatgcaaatggcaaaaatctcagcagctcttcatgttcatcaaaattgtgctaaacttttatatatttcagtattaacttctccaacaaccggtggtgttactgcaagttttgcaatgttaggtgatcttatttttgctgaaccaaaagcacttattggttttgcagggcgtcgagttattgaacaaacgttacaagagcaattacctgatgatttccaaacagcagaatatttgttacatcatggtcttcttgatttaattgtaccgcgatcgtttttaaaacaagctctttcagaaactcttactctttataaagaagctcctcttaaagttcagggtcaaattccatacggtcaacgaggtcctcttacaaaaattcgagaagaacaacttagacgttttattcaatctccaaaaaatccaaattataaacacttagttacagaatttgaagaattacttactgcgttaacaggtgaaaaaacatcaattgactctttaacttctgacaatcttcagaaagcttttgatttagcttgtaacacacaaactcgtctagattggttacaagtaaatgaaaataagataagtattcgacctgtttttaaataggctttttttattcgaattcgtttttaaagatcataataaaaaattttatttaattaactcatcgctttcgattcccaaaaacaatgtttttgggaatcgaaagcgattaaagaaaatctttttaacttttttaataaacaaagaaagaaagtgattaaatataaaatttttactatgagaaaaataatttcaaaggttgaatcaattacatgagtattttagttgaaaacatctcaaaaaaatttggatctttccaagctttagatcgagtaaatttagaaattaaaaacggttcattagtaggtctattaggtccttcaggttctggaaaatcaactctgttaagagttttagcaggactagaactacctgattctggaagaatttggttagagggacaagatgcaacacaaatgaaactgcaagaccgagaaatcggctttgtttttcaaaattatgcgttatttccacatttgactgtttttgaaaatatagcttttggtcttgaggtgaaaaatctagattcattgctcaaaaagcaaagagttcaagaattattaaaattaatgcaactagaaaaatttggaaattgttacccaaatcaattgtctgggggccaacgtcaacgtgttgctttagctagagctttagctgtagagcctaaagttttattattagatgagccttttgcggcattagatgctaaaataagaaaacaacttcgttcgtggctaagagaattacatcatcaaatttcagtaacaactgtttttgtaacacatgattatgctgaagcgatggaattagcacaagaaattgtcattttagaaaaagggcaaattcttcaaataggcacagcgcaagaactctcagatcatccaacaaattcctttgtggaggaatttttggaattaaacaaagactaataattctaaaaaactaaatctcaaatttaattcaaattgttcgtgcctttctaaaaaataacagttgatccctttccccaaacaaagtttggggggaaagggatcgcttttattttcaaattttttttcctaaattaaaaagctcctttttttaacgaataaaataactgagaccagaaacatatagccttttaccggacttgaaccggtgactttttgcttaccatgcaaatactctaccgcctgagttaaaaaggcaaaaaataaaatttattaattttttgaattgatttatttttattctctttcaaagagtctatcataaacttcactccaaatgcaaaaactacaaaaagttcactaataggttattatattaaaagggtaatctcctagtttttttggccttcgtttctcaagtttttaattgaggcttttttttaaatatttagttttccaaaatttttcaagtttgggtccaggttaactaagttttttgaaagaagaatcataaaaaattgaacgattttttttaacattctcaaaaaactaaaatattttaacaactaaagatctaaataaaacatttagatttcattagatttaatagatttaaaaaatttagaaaatctataagtgcttattcaaaatactaaccattatatatgtctacagaaaatgatttaaaaaaacgaactcaaacggattcactaaatccaattgtttcccaaaaagaaaattttgaaaaagaaactcaagagcaaacaaaaccaggttttaatatcaaacagtggatttcaacatataggtcaacaaatcgaattaaatttggtcgaaaattagtacatttttcaactgttctagcttattttcctgtggctgtaggttttttagattttctacgacttcaaattagctcgccacgacaatatgaatctatttttcaaaaaactttccctgtctttgcaaatttgcaaccaaaattgacatatgaaactttccattatgttattggggcaaatttaaaattaaattctaataaaaatggatttgttctaggtcgacctgattttataggaactttgacgaattccaattcggaaaagacttttttaacgcaaataaaaagagagcaaaaaagttcttttactcaaaaacattttcctctaaattcgttttctggtttttcttgttctttttattcgttttcctctggagattttaattcaacaaattcagtaccgcaaattttagatgaattaccaggatacgttcaagggttaataagcagaggctcaaaacaagggaaagaaattcctcctgtcttagtaaaaccaaaacaaaaaccagcattacaaaagactttatcttattctgtgaaatcaatcaaaccatcaaaaaatccgcttttaattccatcaaattttcttttaaattcatcttttcagggtagattacgattactccaaaaatctcagctttattctttgtcaactccattttctttaaaaaaaggattttggtctcaacataatgatttttatactaaccttaaccaattaaataaagaattccaaaatttgtttttaaaaaaacaaatttccttgggtttgaatgatcttttagatcagtcatctggtcccaaaaatgacatttttggtaacgagattaaacaaaattttttagaagaaagtcagattcaaaaagatttaccttttttaaaagattttttagaaaacaattctcatatcgacgatgagcttcttatcgatgtgcttttacgtaatttggataaacaaacaatttcaaaagagactgaaggttttcgacttatgtctggatatcaatatccagatacaacgagtcaagaactttcacgtttttacaagcaaagaggagtttttcattatatttttggtaaaaaagagtggttaaaaaatcttttaaatttttctttagatggttattcagaaaaacaaaaagaaaagcaaatcgtcttaggtacaacaaaatatgattttaaaatacaaaaattaccatcatttgcagttcaaacacaagagacagtagtaaaaagtcgtgaaactgccgaaattttgtatcaaggccgtaatcttgtcttagattctgaaaatgcttttgattggataactcgtggaaaccttcgttcctggttttataattatctatctcctttaaatccatttagtcaaaatccggaaaatttttttggactttatgaatctccatttagttccaaacaaacatcagccgttgcggcttttcaagatagatctctcttctattcatcttttataaaccaaattaaaagatctgctcttgttacaaccccgcttcagagtaattttcttccttttcgctcatcttttgttgttgctcctgatattgaatccaattccaataattttgttcgaggggttgatacaaacttaaccgaaccaaaacaggaagatttagccgctccaattgaatttctaccactaatacaaactaaacaaccttgctttaaagtttcttcccataaaacgactaattttcaagggtattcgcctgtgtttgatattggtttaacgggaaaagcggattacatttattctttagatttagtaacagaaaggtcttcgacaactcattatagttcagggcaatataaaaaaattccatctattttttcaagatcttctgtaaaacgaggagttactaattgggaacctctaacagcaaattcctggttaattattagtcagttaagttttgctgtttttatttttcaaatcctcaaaaatttagcggataattatggtcgagaacttttaggctatttattagatttagttgcagcgttaggatttttagatgaatctttaaaacaagaaattgaacttttgatgggtcgtcgtgataaaggattccgtattattccacaaagttcaaaaacttttaatgatattgtcggggtgcagaaatttctcccggaactttatgaagtcatttggtatcttcgaaattctgcacgtgatttcactttaagtggtgctttaccacgtggtgtgttattaacaggacctccaggtacaggaaaaactcttttagttcaagctcttgctggagaagctcatgttcctgttatcgttttatctggaagttcattaattgaaccgggcgaaatcgcaagtacaaaacttgaactagtttttcaagaagcgagacagattgcgccttgtgtagtatttattgatgaaattgacacgttagctccaaagagaagtggtgttgttcaaaatccaatggcacatgatgatattgttgattttctaaattcctttgagcaatttaacccagactcaacacttgaacgtttagaggctgaagttcaagaagcggaagcagaaagccaaggaaaaatgtcaaatcaggataccagccaacaattaagtttgcttacacagcttctcatagaattagatggtattcgaggacgagatggagtgttggtaattggtgcaacaaatcggcttgatattttagatccggctctcctccgacctggtcgtttcgatcaaattattgaagtagggttaccaaataaacaaaaaaggattgatattttaaagttttacggtgaaaaattaggttatcagataaatattccttggaattatcttggtgaacgaactgtcggatttagtgccgctgatttagctacactaatgaatgaatcggcattaaaagctattttaaatcaaaaagaaactgttcatagtcttcaaacaattgaacatggtattgatcgtcttacaacatcagagactgaaaaaccaacaatcctaaaagtaggtccaacaggcaatctgtctgttgattcaaaactctccctccttcgttttgcctattatcaagccggaaaagttgttgttagctctcttcttaaagagcatccaaagagcgtttttgctgctttatggccacgatgtccaaatatccgatcacttgaaattacgacaaatcttcagactactgtctttgagtttgctagattgtctgaaatcaatgatcgactaattggttgttatgctggaaaagcagctgaatttctctttttagagaaatttgcttcgtatcgttcatcccaaagatcaacattaggcctcgaagatcttctgtttggtcaaacactagcctatgctttacttgaaaaatttttattctattccaaaaaaagttataatcaacaaacaattgctattcctgagaatatcaatgaattagaatttccagaaatgctagaaaaactagcattgtatgaagagattgttgatacaatcgagtatcctccatttgaaaaagcattagaagaagacacaagttcgttaactgaaaaactaactatatatgacgactgggaacctcaatcaaattattttgttccatggtggcaacaagaagcgtctgacgccttagaatttggggagagaaattttcataattggacacgtatgtatttagaaaacccagaacggagttatcgaaatgttgaatggcggccatatgatgaattttatcatacagacactgcgttaaaaaatgtcaaaaccgcttttgctaatttctcgaaacaaaaaattgactcaccagattcagaaattttaactgagcaaaaacaacaaactccagctgaaatagttccgcaactagatgtgtcaaaagaaacatatgtttctgaaccttcaactactgaaattcaagaaacacttaaaactagagatttcacaaaagaaacgcaaaatctcgaagttaaaagtgaggccgaaactgaaacttctactaaaaacgaagtttctgagacaaaggaagaagtttcaagttctcagaaactacaagaagaagctcaaacgaaatctcctagcgggcgagttaaagttccttggcatgatcaagacaaaactcaaattccttatcctgcacaaaacgtctatattccttggagtgatgtctcaaacttaacgcgagattatccaattcattcgttaatactgcaaagttttaataaagctcttataattttaaaccaaaacagagaacttgtagatcgatttgttgttgatttattatcccaagaaattttacgacagcatgaaattgaagccgtgttacaagagtttcaacagttttctacccaaaataagggcgaagatttttctgagcctcttgaaaaaacaaaaatatttgaggttgttgaagctgactggggtcaatattctcgaaaaccacgtcctcgttggatagattttgcacaattttcaaaacaaacaacttagaagccttgatgtgagataaggaactctgtatactaacaacagaaacacaaaaaaaggttcttaaaaacctcgagaagtcgagacgctttgcgacctctttattttgagagtctctcttttttcaaataatttcgattcctctcctttcccaaaaatgtaatttagtcccaaaaacttcgtttttaggaagacactttgtttccaccaaatttcaaaaatttggtggaaaggaaaaggggaaaagattaaatgaaagttattacgactttcaatgacgtaatttttggaaataaaaataagagaaccactctctctttaaaggagattttgtaaaaaaactgaaaattactaaggagtgttaagcatgtctggtgctacaggagaacgccctttttctgatattttaacaagtattcgttactgggttattcacagtattacaatcccatctttattcattgcaggttggctatttgtaagtactggtcttgcttatgacgttttcggaagtccaagaccaaatgaatatttcactgaagatcgtcaagaaactccgcttattacggatcgtttcaacgctttagaacaagtgaaaaaattatctgaagttaattaattcacgaacaactgttcgtaagactgttgacttgtttctttttagaatataaatcaaccttctcccaaaaacgtcgtttttgggacagacaaaactaaaacacagaagtgtcaaacagagagttcctcaattcgtgtgatatcaaaacatattgttttgatactagaaggttttgtcgtactatcatgtagttagaggatatctgttagaactcgattctatcactttcccaaaaaaacaaagttttaatcccgaaaatttcgtttttgggacatcctaaaaaaatacttttttaggtagaccaacttcattttttccaaaatttttcaaatttgggaatgaaatgagtcctccttgaaaattcatagaaaagaccatgactgcaagaaaatcgtatacttatccaatttttactgtgcgttggcttgctgtgcatgcattagctgtacctacagtgttctttttaggcgctattacagccatgcaattcattcaacgttaagagtaaggtgaaattattatggcgaaaccaaatcccaataaacaaagtgtagaattaaatcgtaccagtttatactggggacttttattaatttttgttttagcagttttattctctagttatattttcaactaataaattctcagttcgaacaacaatccaatttttccccctcccaaactctcccaaaacagacagttttaggagagtttatccccattttttaggaaatggggaagggatctatttcattttttgtttttggggaaaaactttctaaattagaaacatagctggtttcgatctcaaaaacttcgtttttgcgaacaacttgttttttggaaagtttcggaaaatttttttgagaaacttctcttttatactaaattttaaaaatttagtataaaagagaaaaactgcttttgctatcccaaaaacctcgaaaaatcgagacgcttctgtcccaaaacattctgttttgggaagacctctcttttaaatttttcaattgagaagagattcttttaaagtaagtaaagtgggaaagcttgaaatttcatttcaaaactatcaggagatttcactatgtctaatactggtacaactggtcgtattccattatggcttgttggaactgttgccggagttgctgttattgctttagtcggtatctttttttatggttcctatgtaggtttaggttcatcgttataatgttaataacttcagtcccaaaaactcaatttttgggaaagattgtgatttttttatttcaaaaaaatcaactcatgaagttctaccacactgtatcggtttaaatccgatcagtgagtttttttctcctcttgaacttgatttgccgctaatcggattcgaaccgatatgacctaaagtcggagctttttgaaagctcagtgtataccaatttcaccatagcggcaggtttctcatttctctttagaggatctttgaagctctaacatataccaaaaattaatcttttctgttgaattaatttgatttaaaggaatttggtttgacaagttttagagataaaaacaattactagtttttttatctctaaaatacatatcagttactgaaaattgttgattccaaaactctaattttaattttctttaaatactatactagcattaaacatttcgattttcaaaatgtttaagagacaaacttaatattaactaattttttgaactgttactgattttgatttttaactactttatcaaacttaggaatcccataattctctaaaaacctaaaaattgaaaaatttctcctaaaaacactagttttgcaactgcaagaaaggctttttctggggtagaaggattcgaacctccgaatggcgggaccaaaacccgcagccttaccacttggcgataccccatttgtaatctaaatgttagtctacaataaatgagcataaaagtcaagttagttttcattagaaattcaagatatttaagccgataaaaggttattttatagaaaaaaaactaaaaatttaacttgtgatgacttaaaaaatttgacttattgttagtttttttttgtctcatagatgtttttttttgttttagaaagaagtttgtaatttttttataaatgaataatggccaaagtgtttgcagaagttttcgaaaatataagaataaataagcctgattttgcaattgagtcattcggaaatgaagcggtacctcgtggtatattcaactcagcgtggtatattcaactcagtccgaaatgactcaattcatgtttttatttataaagtgctactccaaggcgtacagcaaagataccagtagccaatgctaaaaataatgcagtaaagacttgataatcagcgataggcataaaaagactcctgggtttgggtcacacgtattccccaaaatatcatttttgggtggcgcttgttcaaaaaaaatgtttttaaaagcaattaagcctccaattttttttggagagctctttcccaaaaatttcatttttggaacaccttgtttcatttccaaaaattgttcatttttgaatcgatttctttttctagttcttttagaaaacaaaattcgatttatcccttttccggtctcaaaacctttgtttttgggataagattaaacactgtttttcggagagaaactgatacgtgtgtttcacttgctgaaattagaggtttcctccacgactggaaagtaatacaatcacaagcggcccagccgcaacgataaaaagtaaagcagttaattgtaaaacaatttcaagattcatggtttttcgtgtcgcaatgtagttactgagtgtggtttctttcccattctggtttttctctcagaatttaagtaagtttctttctttttactccccagtgccaaaaatagagttggtcccaaaaaattgagttttttgaatggagaagtttttcaaatttagagtaaaacaaagttttaaccgaaaattttttattatgatactagatacaaaaatatttatatctcaaagtttttggtacatggatagaccccaaattttttaaatttgggataaaaggaaacttttagaatgagtacgaataaaattaattatgggaaaggaaataagagaattaacggaaacttactgatgcttgccacacaagagctaataacaagaaaagtactggataataacacgggaatcctccaaaatagtgtatcaattcacaaaaaaagggagagtttaaaaaaactctctcttttgatatttggtattttttataccatttgtgacgtgtttcttcttagttctattatgctaacaaaagagttttttggctagttttttaaatagttttactaagccattatttcgagttaaaaaaaactcttttttgaaatattttcaaatgacttcgtcccaaaatcgacgtttatccccattttctaggaaatagggcaggaatggaattaaaaaaaaactctggccttttttaaatattctgctgtttttgatcagaaaaaagctaaatttttagaaaaacggttgtccccttcacttaaactttgttattctataatcttttttgatgttaaagaggatagacttgagttttgagacatttaaaggcaaaacaaaaaatctttttttttttacaggtttgaggtatactaaacctaagcggatattgcatagtggtagtgcccttgtcttccaaacaagaggtgcgggttcgattcccgctatccgcttatccaaaagcttaaagtttagataagcagaaaatgttattaagtttttggaaaaaatcaacaaaaatttatagaaaaaaattctatagtggcgggtatagccaagtggtaaggcagtggattgtgactccaccattcgcgggttcaaaccccgttactcgcctttgattgttttacagagttttttgtaaagtaaacgtaatttattagtaaattttcgattttttttaattttgcgggtctcaatatttttttcctttcccaaaaatttcgtgtttggagacggtaatccattccaaacacgaaatttttgcggcagactctcttttttttatttgtttcatctaaaattcgaacgggtggttattctcataagatttagaatgtaatatttaaaaaaataagtaaaagtaaaaataagaagattaaaatccaggtactccaaagtaaaaaatctttagctttactataatttgtaaaaagacctgattctaataattttcgaagaacaatattttcggttggtgtttgtggtgctattaaaataataagacagaatgcaataagaactgaaatcacatgtaacataaatacaaattactccttttgataaaagtgtgttttgaaccactagcttatcactcttcgaaagttatggtacaatgaatacaaaacccatgccgggatagctcagttggtagagcagaggactgaaaatcctcgtgtcaccagttcaagtctggttcctggcaatttgatacctccttttcttttttacttttcgaaaagtcgcatttgtctcaaaaatttcatttttgagacacctctttcccaaaaatttcatttttaggatggtttaaaaaaaggaagagaaatttttgatttttaacattttttctttgtctcaaattgaaaagctagcccaaaaattttctttttttgggaaatttgttgttttaactcttttggttcaaattagactacacaaaaattcctagttctatgattatatcatagataaacaaaccctttttgattttttaaaggagtggaataattagaaccttatagttaaaatttctaattaaaaaattttgttattgctcttatatgaaacaatttgtagctgaaacggaatctattctaatgaatcaaaaaaccctgtcaaaaaacagagttttatgtagtttttcaggtggacaggactcaactattgctttttttgtcttatttcatagaaaaagacttagctgtcaaaccattaatcttctttattttcaccacttttggcaaattcaaaattttcaagcctctaaaggaatttttcatttaagttttaatttaaaagtaccttatacgctaatactgtcaaatctctcgttagaaaatgaaaatcggtcaaggaactggagaaggaaaaagttgtttaagttttacaatctgcagcacagttcactagttgttactggacattctcatacagatagagttgaaacaaatttgaataatctaattagaggaacaagcccgaaaggagtttcttctctaaaaaattttggaggatttccagaagcaagttttaatactcctcaatatttcacaaatagttgcttttgttctttagtctgttcaacttcttttgtgtcttttagattattaactcaaccacaacaaagaaaatttgtaaataaaagtaaagtcagatctaaaatctttttttcacctaacaattcaggttttgaaaaatctatttctctctttttacaaggtaaatttgtcagaaagtctaggttgacaaatgaaaaatcgccgagttttaaccaacataaatggcagattattttttgtaaaaaaaaattgagatgtcatcctaaagatatttttttaggaagtcgattagaagagagtcaaaaaaaatccactaatttgtggttttgggaacaaaaaaaaattggtttaacccaaaatgttcaaaataataattttttctctatcttttatcacaatcgtaattctattataaattcaaattctttttgtttttcaaaattttttgaaagaagaatagttaatttaaaaactcccattaaaaatgagactcgcgttactgtttcaaaagtagcaaaaatttatagtttaccggtttttcatgatctaacaaatttttcttatcagtcttctagaaataaaattcgacatattgtgtttcccgtaattggctatctttttcagaaaaaggtagatttttcatttacacaatttttttcaactctaagatatgatacttatgagggtgataatacttctcaaaaattttattttcttcttgagtttttttctaaaccaagttttttttattcaaaattggttaaaaattctgaaaatctgtctactccaaaaacattgtttttgataccagaaattcttggagattggggtgtttttgtaaaccaaactaaaatgaagagttttgaaagttttcttttactatatgtttgtcgaaagagtgatagaatttttcatccttacattataaaaaaaataatgcgcaattatactgaaagagagatctcattttatcagattacaaatattcaaagcattcttaaagtataaagaaaatttagttaaaaaagaaggtaaattaaacgaaaatactttaatagtttaaaaaaatttgggttacccgggactcgaacccggaactaatcggttaaaagccgagtactctaccattgagttagcaacccgaaataaaataaatgagattttcttttgtttatctatttacttctgtaattataacaaattacagaaagctgctcaagaataattttgctatttcaccaattttggtccaaaaacacattcggaccaaaattggaaaagagtgaatttttaaacaacaaaagaattaagaattcctactaaaaatacaaggaggaaccaaattccaattcctgaaaatactgtgcccttattttcagcccagccttctggtgttgctaaaacaacaggcacaccaacaactaaaagaaacgatacaacaatgaacgcaaataaagctaattgaaaaattaaaagcatagctttgatttccttaaaatggtctttttttactagaaattgtcttgtaaaagaggatctttttcgctcttagagttctaagaatgaaaaactttgtatatacttttagtataccctatcagtcaacgaaaacttagtatttttcaatttgtttaattagctttccttttgaagtaattggttttctaatgtaccattttacattttaattttattagtaatttagtaataaaaaatataaaaaattcaaaaatttttatattttttatttttttatttttaaacctcttgttatactaaatctaatcgacactcttttgatttgtttttgatgaaaacgttttaactctatttcttttttcgagaaataaaacaaaaaataaaattagtcgatattttttattattttttaattatttaccactgataattctaacagagaggatactatgaaattagcttattggatgtatgccgggccggctcacataggaactttacgagttgctagttcttttaaaaatgttcatgcgattatgcatgctccgctaggagatgattattttaatgttatgagatccatgttagaacgagaacgagattttactccagtaacagcaagtattgttgatcgtcatgttttagctcgaggttcacaagaaaaagttgttgaaaacattacacgaaaagataaagaagataatcctgacttaattcttcttactccaacatgtacatcaagcattttacaagaagatttacaaaattttgtaaatcgggctggtttagactccaaagccgatgttattcttgctgacgtgaatcattatcgtgtcaatgaattgcaagcagctgatcgaactcttgaacaaattattcgtttttatttagaaaaagcccgaaatcggggtgaattagacacagtaaaaaccgaaaaaccctccgctaatattcttggggtttttactcttgggttccataatcaacatgattgccgcgaattaaagagacttttaaatgatttaggaattaatgtgaatgaaattcttccagaaggaggtttagtaaacaacattaaaaatctcccaaaagcttggtttaacttagttccttatcgagaagttggtttaatggctgctgattatttaaaagcagaatttaatatgccttatgtttctgtgacaccaatgggtttacttgatactgaaaattgtatccgagaaattattcaaattattaaaactaataatccagaatataattttgattttaattcttatatagatgagcagactcggtttgtttcacaagcagcctggttttcaaggtcaattgattgtcaaaatttaacaggaaaaaaagctgttgtttttggagatgctacacatgcagctagtatgactaaaattttagctcgtgaaatgggaattcgtgtttcatgtagtggaacttattgtaaacacgatgctgattggtttcgagagcaagttgatggtttttgtgatgaagttttaattactgatgatcatactcaagttgctgatatgattgctcgtattgaaccagcagccatttttggaacacaaatggaaagacatatcggtaaacgtttagatattccatgtggagtaatttctgctccagttcatattcaaaacttcccattaggttttcgtccgtttttaggctatgaggggactaatcagattgctgatcttgtctataattcatttactcttggaatggaagatcatcttcttgaaatttttggtggtcatgacactaaagaagtaattacgaaatctttatcaacagaagctgatttagtttggacaccagaggctcttgctgaattacagagaattccaggatttgttcgtggaaagattaaaagaaatacagaaaaatttgctcgcgacaagaactttactgaaattactcttgaggtgatgtttgcagcaaaagaagctgtaggtgcttaagaaatttatctgagttttaatcaacgttgttttttcttttttcaaaacaatgcaatttttattaacgtaaaaacaggtattttttgtcccaaaaacaaagtttttgggacatcctaatttagttgatgtccttttggggaaaatgttttttaaaaaatttactaccatcaagaatcttattctatttaacttttaatactgattttattttggttattactaacatctttcatatagtaagtaaccctttatgctctctattcctatggaattctagatagaaactctcacttcccaaaaacaaaatttactccattcccaaattgaaaaatttgggataaaacaaactgttttcccaaaaccgttgttttttggaaagggaaagggacgagtcaaaagagttaccagaaatcgttcttgtccccaaaacaacatattttaccccaaatttcaaaaatttgggaatggatgaaactgttttctggtaattttgtacactacatgtttcaggtacccttatcagtaaagggtcattttgtgaaatcattagttcttcctaatttttcttgaaaaattaggaaaggtcatcccaaaaattacatttttgggacacccttatcccaaatactttgttttaccccgaatttcaaaaatttctcccaaaaacagtgtttttggaaatggaagggagaaaatggtttttttagggataagtgtctttttcaaaagtttttggaaaagagaacgagtgaattcgagaattcaaataaggagaatttttcgccatgacaatcagtcctccagaacgtgaagcgaaaaaagtcaaaattgtagttgatcgtaacccagtcgctacaaattttgaaaaatgggctaaaccgggtcatttttcacgaactctgtcaaaaggtccaactacaactacatggatttggaatttacacgctgacgcacatgattttgacactcaaacaagcgatcttgaagaaatttcaagaaaagtatttagtgcacattttggtcagctaggtattatctttatttggttaagtggtatgtatttccatggtgcacgtttttcaaactatgaagcatggttaacagatccaacacatattaaaccaagtgctcaagtcgtatggccaatcgtaggtcaagaaattttaaacgcagatgttggtggtggattccaaggtcttcaaatcacatcaggtttcttccaactttggcgtgctgccggaatcacaagcgaattacaactttacacaacagctattggtgctcttgtcatggctgctgcaatgttttttgctggttggttccattatcataaagctgcaccaaaattagaatggttccagaacgtggaatctatgttaaatcaccatttaggtggtcttttaggtcttggtagcttagcgtgggctggccaccaaattcacgtttcattaccaattaataaacttcttgatgctggtgtagatccaaaagaaatcccgctaccacatgagttcacgcttaatccagaattaatggcacagctttatccaagtttttcaaaaggattagcaccattttttactcttgattgggctcaatacagtgatttccttactttccaaggcggtcttaatcctgttacaggaggcttatggttaactgatactgttcaccaccatcttgctattgctgttcttttcttaattgcagggcatcaatatcgtacaaattggggaattgggtcaagcttaaaagaaattcttgaagcccataaaggtccatttacaggcgaaggtcataaaggactttatgaaattttaacaacttcttggcatgctcaattagctattaacctagctctttttggttcactttcaattattgtatctcatcacatgtatgcaatgccaccatatccgtacttagctactgattatggtacacaactttcactctttacacatcatatgtggattggtggtttctgtattgtcggtgctggtgctcacgcagctatttttatggttcgtgattatgatcctacaaacaattataacaatcttttagatcgtgttcttcgtcatcgtgatgctatgatttctcatttaaattgggtttgtattttcttaggtttccatagttttggattatacattcacaacgatacaatgagtgctttaggtcgaccgcaagacatgttctctgatacggcaatccaattacaaccagtttttgcacaatgggttcaaaatacacatttcttagctccaggctttactgcaccaaacgctttagcaagtacaagtccaagttggggtggtgatgttgttgctgttggtggaaaagttgcaatgatgcctatttctttaggaactgctgatttccttgtacaccacattcatgcttttacaattcacgttactgttcttattcttttaaaaggtgtactctatgctcgtagttcacgtttaatcccagataaagcaaatttaggttttagattcccttgtgatggcccaggtcgtggtggtacttgtcaagtttctgcttgggatcatgtcttcttaggtcttttctggatgtataactcaatttcaattgttattttccactttagttggaaaatgcaatcagatgtttggggaactgtaagcgcaaatggtgtttctcatattacaggcggtaactttgcacaaagtgcaaatactattaatggttggttacgtgatttcttatgggcacaatcttctcaagtaattcaatcttatggttcagctttatctgcatacggtttaattttcttaggtgctcactttgtttgggcatttagtttaatgttcttattcagtggtcgcggttattggcaagaattaattgaatctattgtttgggctcataacaaactgaaagtagctccagctattcagccacgtgctttaagcattacacaaggtcgtgcagttggtgtagctcactatctattaggtggtattgccacaacatggtcattcttcttagctcgtattttagctgtcggttaagttttgccctttttccgttcccctcccccttcccccaaacagtgtttgggggataaacagggtttttggggtacattttaccaccctaaatttttgaaatttgggctctcaaagttttctctttttgattcaccatatcccccatactgtgtttggggaaagataaactttgattttttttacgtttcgcatccctgtttttgtgaaaacagggtaagcattcttttctcaaaaaactttctatttgagttctagttttttatttctactttatttgagtaaacaatacattacccttttgggaggtttagtgatggcaacaaaatttccaaaatttagccaagcactcgcacaagatccaacgacacgtcgaatttggtttggtattgcgactgctcatgattttgaaagtcatgatggcatgactgaagaaagactctatcaaaagatctttgcttcacactttggtcaattggctattatttttctttggacttctggaaacttattccatgttgcatggcaaggcaactttgaacaatgggttcaagacccacttcatattcgtccaatcgctcacgcgatttgggatccacattttggacaacctgctgttgaagcatttacacgcggtggtgcttcaggaccagtaaatatttcaacatctggtgtttatcaatggtggtatacaatcggtcttcgtactaatcaagaattatatactggttcgattttcttactcgttcttgcagctcttttcttgtttgcaggttggttacacttacaaccagcatttcaaccagctctttcttggttcaaaaacgcagaatctcgtttaaatcatcacttagctggtttatttggtgtaagctcattagcatggactgggcatttagttcacgtagcaattccagaatcacgtggtcaacatgttggttgggataatttcttaacagtattaccacatccagctggtttaacacctttctttacaggaaattgggcagcttatgcagaaaatccagatagtgtgtcacatgtatttaatacagcacaaggaagtggaacggctattttaactttcttaggcggtttccatcctcaaacacaaagtctttggttaactgatatggctcaccaccatttagctattgctgtaattttcattttagctggtcatatgtatcgtacaattttcggaattggtcatagtatgcgcgaaattcttgaagcacaaactcccccttcaggtagtttaggtgctggccataaaggattatacgatactgtaaataactcgcttcatttccaacttggtttagcacttgcaagtgttgggacaatttcttctttagtagcacaacacatgtattcgcttccaccttatgctttcttagcacaagatttcacaacacaagctgctctttatacgcaccaccaatacatcgccggatttattatgtgtggtgcatttgctcacggtgctattttctttgtacgcgactatgatccagaagcaaatcgaggaaacgttttagctcgtattttagatcataaagaagctcttatttctcatttaagctgggcaagtttattcttaggtttccatacattaggcctttatgttcacaatgatgtagttcaagcttttggaactccagaaaaacaaattcttattgaacctgtattcgctcaatggattcaagcagctcatggaaaaacagcatatggttttgatttcttactttcttcagcaacaagcgcaccaagcttagccggccaagcgctttggcttcctggttggttacaaggtatcaacagtgatgcaaattcacttttccttacaattggccctggtgactttttagttcaccacgctattgccttaggtttacatacaacaacattaattcttgtaaaaggtgctcttgatgctcgtggttcaaaattaatgccagataagaaagatttcggttatagcttcccttgtgatggcccaggtcgtggtggtacttgtgatatttctgcttgggatgctttctatctcgctgtattctggatgttaaacacaattggttgggtaactttctattggcattggaaacatcttggtatttggcaaggaaacgttaaccaatttaatgaatcttctacatatcttatgggttggttacgtgattacctctggttaaattcttcacaattaattaatggttataacccgtttggtatgaacagtttatctgtatgggcttggatgttcttatttgggcatttaatctatgctacaggtttcatgtttttaatctcatggcgtggttactggcaagaattaattgaaacacttgcttgggcacacgaacgtacaccactcgcaaatcttgtgcgttggcgcgataaaccggttgctttaagcattgtacaagctcgtcttgttggtttaactcacttttctgtcggctatgtattaacatatgcagccttcttaattgcaagtacaagtggtaaatttggttaattcctctaatcattagatgttctttctcttgtcccttgtctttccccccttttgacacaaaccgagtttgtgtcaaaagggggaaaagacaagggattgggtttttgttataccttcaactttttcgacaaaatttaaaggaatggaaacaggattaattgaattatacttctaaaaaacattttttagttttgatagttagtattaaaacgtttttaaagtacttcagttttgttaattaaacccgacttaataacaaattattatgtccttttcaaaaaaaaattttaaattctgtgtttagttttaaagctaaacacagaacaccttcgtgaataccacatcgttgctagtataaaacaaactctgattcctttttaaacgaatttaatcgctcgagtcggacgaaataattgccgagttgcggccctttttcaaggatttcaatttcccagtccttttcccagtcctggtagttttgataatcagccacaagagcggtatttataaaagaaccttctctcagtctctcaaagaaaattaataggtcaacagctaaacaagccacttgatcataatatgcttttttctttggacggcaaattaggcggaaaataccaggattcgaatctgcaaattttagcgaactatttttgttttccatataatgagtgtgactaaagagataactccgatggatttgtttttttttctttctctttcattttaccataaaacaaaagttttttaacgacttttgctttcaaaaaaacagtcattatctgttaaagtcttttaaattaaaattgcttgaaagtattttaaaaattcttaattttttgctgttgaataagaaagtgaatttcttcttagtagcgtctcttttgattgattactccaatttgacttagtcgagttttttaattatcagatagctagtcttttttcatctcatttttcttacaaaaaaaagtgatttccttgactcgaaaggggatttttgaaacttcttttccgtcaaaaaagcatttggtaaagtttcttatcttttaaaaaaagataaatacaaacaactattaagaagttttctctcattttcctccattctcaaatttgaaaaatttgcgagaaaacagactgtgacaaaaaagttaatatggtgattttttatttttttgtcatcatctttttcaatgaaggtaaaaattggccgaaattaattctgaaaaaaacctaaaaggctttgctagagaatgaaaaccaaaaatgactcattttaatctaactggacttacgttgcaaccctgtccacacttcaaaacaaggagactaagttgtttcaattttgtatttcaatatgcactacgtgtttcaaaaaaggaccaagtttacttatttgagttagaaaacgcgattatcaaaaaaattgatttagaagttttggtttctaactagctggaaagattttctcttttgtatattgaagtccaaaagaaaaaaattttaaaggttttttgtcaaatctccaagcaaaaaggagaaaagctggttgtgtgtttgagatttgtgtgtctagaaaatctttttttacaaaaaacacaaaaagcctggcaaaagccaggctttttgtgatatgaactttttcttagaattaacaagaattagccattaacagctggagcttcaacaacagctaagtctaatgggaagttgtgagcgttacgctcgtgcattacttccatacctaagttagcacggttgataatgtctgcccaagtgttgattacacgaccttgtgaatctacaactgattggttgaagttgaaaccatttaagttgaatgccatagttgaaatacctaaagcagtaaaccaaataccaacaactggccaagcagctaagaagaagtgtaatgaacgtgagttgttgaaagaagcatattggaaaattaaacgaccgaagtagccatgagctgcaacgatgttgtaagtttcttcttcttgaccgaatttgtaaccttcgttagctgattcgttttcagtagtttcacgaattaatgaagaagttacaagtgaaccgtgcattgcagagaataatgaaccaccaaatacaccagctacaccaagcatgtggaatgggtgcataaggatgttgtgctcagcttggaatacgatcatgaagttgaaagtacctgagatacctaaaggcataccgtcagagaatgaaccttgaccaagtgggtagataatgaatacagctgtagctgcagcaactggtgctgagtaagcaactgcgatccatggacgcatacctaaacggaaagataattcccactcacgacccatgtaagaacatacacctaagaagaagtgacaaacgataagttggtaaggaccaccattgtataaccactcatctaaagatgcagcttcccaaattgggtagaagtgtaaaccgatagcgttagatgtaggaataattgcaccagtaatgatgttgtttccgtataataatgaaccagatacaggctcacggataccatcaatatcaacaggaggagccgcgataaacgcgataatgaaaacagaagttgcagttaataaagtagggatcattaatacaccaaaccaaccgatgtataaacggttttcagtgctagttacccactcacaaaaacgagcccaaaggctagtgctttcacgtctttctaaaattgctgtcataaatttaaaatgttttttcttttgttctttttttccacaaaaagtggaaagaatttttcccaaaattaaaaaagtttttaacttgttactagtagtatacgcgaaaaaaaaaatcaaaaacaagtaatttttttatttataattaacatataattttatgggagtctaatggaagttttaacttactaatttaaataactaaacaacgtccccttttttgctttcccaaaaacgaagtttttgggaaagggacctgttagcctacaaattttgtttacccaaaagttgattagcctctccaacttatgcacaaccaaagtgttttagaaattgggataaatttgtatcaggaaaaaagtggggcttagattcaaagagagtctacgcttgtagctcacctgtttttaaaaacttttgtggatgtttttttcatttcttcttaaaaatgaggctatcttgtttcgataaaatgtaggttatttttagaaaaaaaaaaaacggacttttaagataaacaaatacagttttccagaatcaagactgatttttgttctctggaatttcaaaaagaagctaccttttcgcgtttcttcacgcaagaaagatatctttcttgcgtgatggatttcttttttagttaatttgattagggaaatgagaatctgaagcaactgatttttcttgttgatcttgaacaacttggtcgatttttgcaactggactatctaaagtagttggtgtgagtgtttttttcttttttggaacattgataatacccgttccagcaggaattagttgaccaacaataagattttcatgcaggcctctgagaaaatctgtctttttagcaagcgcactttgaaccagaactttgcttacttgttgaaaacttgcagcaagaaggaaactttcagattgcaacacactttttgtaattcctaaaattacaggttcgtattgtgctggacgacaattttgttgttctaaaacgttgttgattttttctataagacgatgttgaattagttcgccaggaagcaatccactattaccactttcgacaattctcaccctagctgtcatttggcggacgacaacttcaacatgtttttctgcaatagaaaccccttggtttgaataagcttgaagaatattttcaacaagaaatgattgaatatatcgaatacttaaatgaacagcctctcgatttcctcgaactcgacttgcgcgacggaaaaaattatgaagaagcatatgcatattattatgaataatttctccgccctgcgtttcacgcgcttcgaagagttgttcaatttttggaataccttgaacaatatcttctgtttgcaggcgttttgacctcaaggttactaacaattgatttttctcaacaaaatcattattcaaaacatgaacaagtccgcgaagtgaagctaaaagtgggacaccttttcgaactgtcaagcttttttctgtgattttagtaatttggccacttgtaggaaaagcaaagtttgatccccattcctgaccccaacgtctatggagcccaaccctattaacttgtttttctctttttatgtccaacatcgaagttgtcggtttagatgtgtttgttttctcaaaagacttagagagttttaaattttttgattgtaatagaggaagtttaaaggtaagaaaatcactttctcggcttaaacttaagcttatatttgtttgtttatctttcttttcaaacccacgaaattcaccaagtgtttgggccttaaaaaaagcttgagttataggaaattctggaattacccagccattttcaaaagtttgaaaacaaaattcatttgtgtcttcaaaaatctgttttgtataaacaaaagcattccattctggaataacaagctgaaaatatttttcgccttcattttgagctgatttaagacatatagtctgatgaatttgttcgaccttattaaacggagccaaagattgcattgtaatttcaatattaagtaactgcgatggcaaacaccatgttgatcctttaatgctatttgcagtgaatttagccactccaaaacttcgatttttggaaaactctcgtgttttttcttgttttgtgaacaatggcgatctaattgaaaacggtttagttcgattcgtaacttcaagccatctttgctttaaaacttttttatcaggtaaaactttaacattacttttttgaaaagcaaaaatcaaacttttgagtcgtttttgttccaaccaaaaaacccactcttttaaactagattttctacaaaaacaaaattcagttttcaagtttttcgtaaaagttttgaataaaggggttttaggaaccctatggaaagtgaaactagtttcaaatttttgattaagttttaaaagctcattaaatctgtaaaaattgttaaattttttaattttttcttttgtttttgcaaaaagaaaattctgttcttttttcttaaaaacaacaattttggaattttcaattttagaaaaagaaatatatgaattaggaaaacaaactttttcaataggtttacctggtttaagaagaatctggtaagaaggtaactgaattggattagaaacccccatttctgacacatagagccacagattttctttttgcaggattttagtttcataagacgctaagagttggtcttgaacttttctgaattttcttttaaaagcgccgctaaagatgttctgatttttttgcaaaggatttttttggaattctctgagacctgatagacttgaacggtaaaattgaatagagcctgccgacttgagcgtcaaacttctgtttgatcctctgtttatctcattttgacaatagttttgaatttgaaaaacacctgatttggaaaatagaccattagctagaataaaaaaagatagaaatttttttgaatcattgaggagaaactggttttgataagtttgaagaaatgagtaaaccttagatgggcaccaaacataggcgttagccagacaagcttcatttgagaaaggaaaatcaaaaattggataagtgtacccaacttctcgatgttgatgttctgaaaggaatgggtaccaagtcaagagtatttgatctactttagatgtcgaaacaacaatatctctgggttttaaaactggtttaaaaaaataagatcctccagaatagtgaatttttgaaaaaacaaattgtgttacagaagaaccagaaataagagaagattttaatttttttatttgtactttcgagcttaaatgaatgtttatttggtgaattggagtttgacgtgaaacaagatcacctttttgaaaaaatgataaagaccgggttattttttcttttagaaaaaatgataacaaagaatttggttgtctttggttccaaagaggatagttttctttttggataaatgaagacataatccaaaaatttcctaaattgattaaagttggaataatagggacaacttctttttcactgtcaatttttttagaagatggttttttcgttttatctttacgtttctttaactgagtttctcgaattcgcataaattcaaaaaaaacttcaccagaaaaaggtgaaattacaggatgacttgattcaggcatttcctgtttttttgtttctaaatttaacgcttgaattaaaagggcacctgtttcgactttctcaccttgtttgacccataaaagactccctgctggaacatcaccttgttgaatttcataataagagggtgaagccgaactttcagattttaaaagacgaagaactggtttttttgggttactaatttgatgtttaactaaataagcgatttttccatgcggagttcgaacaaaaacaccagggagtgcttcaggaaattcaacttttcctcgaaaaggagctgtaaaagattttacagattgttctgaaaagaccccaacaccaccagtatgaaaggtcctcatagtcaattgtgttcctggttcaccaatagattgagctgcaataattccaaccgcttcaccgatgtttacaagcttgccttcccctaaatcaagcccataacacaactgacacacagatttttctgtttgacatgttagaggagatcggacaaagactttactatatttggaagcaattttttttgctaaacggggtaaaataagtgtatttttttctagaggaaaacttggtgagaaatctaaatcatgtaaaagaactctaccaataaggcgttgttcgagatttttttctttaaggagaatcccttgtctagtttgacaatcagacacacgaattacaacgtgttgaacagcatcaactaatcgtctagttaaataaccagaagttgctgttcgtaatgctgtatcaactaatccttttcttgcaccataacaagaaattaaatactcagtgactgttaagccttcacgaaaattactttggatcggaaactctacaatagcaccttgaggatcagccattaagcctcgcatagcaactaattgccgcacttgcgagatatttccgcgagcacccgaaaaagccatcatatagaccggatttacaggatttgtaattcgaaaatgctgaatagctttttcacgaagggtttcacttgtttggttccatgtatcaattaattgttgagatttttcaactgttgtaagagttcccgtcattttaactgttgttacagtgcccattttacgagatgcttgagataagagtgcagctttttgtggtgggatttgtaaatcatcaagacctaatgagacaccagcgcgcgttgcttgatgaaaaccgacttgttttaaattttcaaccaaatcgacagtttgtttttcaccgtaagtttctaaaaaccaagctattaatgttttcaagtgatttttatcaaaacaagcattaaaaaaaacagtatttggctgtttgttatcaacaaaattatttttttctccactcttttctccatttttgaagaattttgtttgtgagcctagtttttttcgtgaattagaaaagccaatttgctgaggagaagctggaaacgaaaaatttttattgttttgcatagtcatactataatttcctcttaagtctgtttttaagatttgtgtttctctgtttggttttccttgggccttcctctttcccaaaaagaaagttttccctcaacttttgaggaaacaaagtgtcttggagaaggcgataaacagaggtaatcctgtcctaaaaacagtgtttttccccaaacaaagtttggggaaggggttaacttgtatttttgggaaagtaaatggaaagagacctgattaaactatattttttcatagcacagggtgaaaatacaaataaaaaaaactatttttagattagattctaaaattcttgcttttctttttgttttaagaaacagaacgactactattttcaaaaatccaagaatgcattaaaattcgacctactgtagtacgaacatactgtgaacgaggagcaagaattggtgtttcaccagaaatgagtactgtttcaagaaaaacattttcaatttttccaaaaacagtaacacgagtttcaataacattttctttagtataagcttggtgttctgaaacaaagttttgtacaaaacctggccattttacccaaacaatactgtgtaatttaagaaagccacgatcatatgatttttttacttgcgaaaaacttgagaaaaatctttctttagttggcccaagagtttcgaaaacagttcgtttttgaagattttgactttgaattaaggcttggtgacttggaatcaaatactcattatcttttgaagaagagttgctttttgttcctttcttaatttgaggtagttccaactgactagatgttgtttcatttttcaaaccttcttgctgaaatttttcttgagaacaagttaaataataaaaacctaataccatatcttgtgttggaagaagaagtggctgacccgaagctggtgctaaaacatgatttcttgaccataacaaagttaaagtttcagctcgtgtttttggtgaaagtggcacatgaacagccatttgatcaccatcaaaatccgcattaaatgcaggacatactaaaggatgtaataaaatagctttaccttcaacaagttgtggtaaaaaagcttgaattcctaatctatgcagtgttggagctctgtttaataatactggatgagtttttaaaatttcacttaaaatagaccaaacaatgggtttttgatcagcaattaaggtttttgctgctgttgttgttactacaattcctttttttcggagtttttgaattataaaaggttgaaaaagctcaagagccatttgttttggaagaccgcattgatgaatttgtaattgtggtccaacaacaataacagatcggcctgaataatccactcgtttgccgagcagatgttgtcgaaatcgaccttgtttaccttttaaagtatcgaggagagattttaaaggtctacccgattcaacacttcctgttttaagtaattcatcaacagcttgttgaacttgccgaaggttataacaccatgatagccaacttccacttaatgaagtatcgaaaacgccaaaatgagctccttgtgtaatacgtttattacgaattaaaacttttctatatagaacattaatatcagaaacaacaagttcgccacgaattgaggtaattggtcgtaatcctggaggtaacacaggtaaatagcttaaaatcatccaagcaggttgcatttttgttcgataaaaatcgcgaaacagttctcgttgccgtttacactttagtttcatggtttttaatcgattgatttttattcgtagccgtttatattttttcctgtcttcataagatctgaaatcaagtgctgctattttttcaaggttaattcgaatagcttcatttaatcgtatatcatgaaagataagctcagattgaattttacgtgcatctaaatgagaaagaatatgctgaaacacaaacccaccagtttgtatgccataagattgtctatgatgtggaattttttcattatgattttctatcggtaaagtaaaagcataataaggaagacttgattcataacgaaaaggtttttcccacaaataaaataagatttcttgaaattcttcaacatgtcgccatgttgcatcgtaagcaaaaccatagagtcgtggttctccactgttcatgttaaacagaaagcgtggatatttgagttctttttttgtagaaaataacctcggcatttttaactttgttttttctacaaaagttggttgtttctccccaaaactttgtttgcgggaacgggataaaaagtctaaaacaaggccacgtgggtatttttttcgtttttcaaaaagtagacttgtttgccagcgtgatcgaaaacgtgtaaacttttgaggaagactacaaaattcagttgttaacatcaccgcttgaagacgacgtgttgaccaattcatacagagacttattggagaaggtctttgtgcagcataaagaggatgaacaataggttgttttaatttaatataaccaagacgatagcgtcttactctagataacgttcgttcaacgccacatttaggacaaactagttcttttatttttgaatcttttgaactttttttaccacaagaacaggtgaaatcgagaactggaccaaaaattttctgacaaaataaaccaccagcctctggctttaaagttttataattaactgtttcccaactcgtaatttcaccgactttctccccagacggtaaaaatctttctgcccactcgcgaatttcatttggtgaagcaagaccaatttcaattttatgaaacagttgatgtggttgtttatgcatatgcgtctccgttttgattactaattttttagttttgtttgtttcttccttttcccttttcccaaaacttttttgaatcccctaccctttttgcttttcccatcccacaaatgaaatttgtgggaaaggaaagcagggattaaattaaatagagttcaattctaaaatctcttgtaactatccgcctcaacacaaaaagttttaggacgagactgttccaaaaacttcgtttttggatggcaaccagattcttgtgcaaattttctgagaaaaattcaaaatttcttattggatttgaaattttgaattttgggaacgcaaaacaaacattaagtaactgttttttgtgaaacaaaaagaaaagttacctattcctaaacaaatttttttaggctgatcccaaaaatttcatttttgggagacccgctatgctttttgaacagtatgctgattcagaaaatcaactctattctcttctaaaaattgtgtaagtttccaaagagaaaaaggacgctgattttgaggctgtttgttaaaaccataaagttgaagatcaaagcataaagcttgtaactctcgaagtattaatttaaagctttctggtcgaccaaaaggcaaacgtttattggcataaatttgaaaaacagcttgttttcttccttcaacatcatctgattttaatgttaataattcatgaagtgtttgagctgctccataagcttgtaaagcccagacttccatttctccaagtcgttgtcctccatttcgagaacgacctcttactggttgttgagtaacggctgagtaagggccagtagagcgagcatgaattttatcatcaacaaggtgaactaattttaaaatataagcacaaccgaccgtgacaggttgatcaaaaggcagacctgttcggccatcaaaaagtctgattttacctggatgttgaggttcaaatagccacgtattaccggttttgactgacgcttcataaagttttgagtacactaaacttcgtgaagcttcagcaccaaatttttcatcaaataaatgtgtactataagtttcatttaagtatcggcctgccaatcctaaaagacactctaaaatttgaccaacattcattcgtgaaggtacgcctagcggatttaaaacaatatcaataggtgtaccgtcaggtaaatagggcatatcatgttgtggtaaaatatttgaaacaattcctttattaccatgtcgacctgccattttatcaccaacttgaattcggcgtctctgaagtagaactattcgaacacgtaagatcgcatctttttcagccaaagcagcaacatcagcctcacgagatggcaaaatttgaacatctaaaacaaagccttcaacgcctttagggacacgtaaacttgtatttcgatattgtgttttttcgcgttgcataatcacgttatacaacttttcatattgttgttctacagaagggccttttggatttaatggactaaccttccctactaaataatctccttcttcaacccaactgccgattttaataagtccttgcgaatttaatctatcacgtttacgaatatctcttgttgattggtcaattttgagtggaatttgatttgttatcttttcaagcccatattgagtatttttaacttcaatatcataatgatctatatgcaaagaagtaaaaatatcctgattaactaatctttcactgattaaaatagcatcttcaaagttatagccttcccatggtaaataagctactaaaatatttttaccaattgacaatttaccttgggcactagcagcaccatctgcaagaatatcacctttttctacccaaataccttcagaacaagatggtcgttcaaaaaaacatgtactttgatttgttcgttgatagttttgaagtttataatttttttgatttttggaacttttattttttgaaacaagtgaatcagattcttttgttaacaatgaaaaactttcaattaatgtgaaatttttcaccaaatttttttctttagtattaaataaaaaacttgaaaggttttcgccaggtagatttgtatacgttttaaatttttgagaaaaggaagaaaaccgaatagtggggcgtacaagtccatgtattcgttttttatttagaaaagaaattttatgaatttttgagtaggccatttgcgatttatcgacaacaccaaaattttcaattccttctgttgtttgcccaaaaatttgatttttgaatgtaatttcgtttaatcctaaaaactttgtttttaaggccggattatacttcgttttatttaactctaaaaatatgggtgttttatctattttagagtcaattgaaaaattgagtattgaagtttgttccggtttcacttttttaaaacaattgacggtgttttgaaaaaacgaactcgatcccaaaaattttgttttaatagaagggcttaaagtttgaatttgttttgatgtgacatttgtaacataaccactctgagatgcttgaataacgtgatttacatctgaaacaacacgagtttctaatcctgtcccaaccagaggtggttctggatttagaagcggaacagcttgacgttgcatatttgatcccatcagagctcggttcccatcatcatgttctaaaaaaggaattaaactagttgcaacagaaatggtttgtaaaactccaactgcttgtgttgtaatttgagtcgacttttcataatcaaatttaaattcttttctgactggaagaagagtatttggtaaaaaattccatgaattcagagaaatatcagctggagctgtagtaagaatctcttcttggtggggtgctacaaataatggtggcaattccttttgaacttgtcctttataaacttgaaaaaatggagttttaattgttccaaaaagattttttgatggttttgcaaatacagtaaaagaatttacaagtcccgcattttgaccttcgggagtttcaattggacataatcgaccataaaaagttggatgaattccacgaatttgaatggttgtttgtttgctagaaacgcctccaggtcctaacactgtaaggcgacgtttatgtgttacttcagccaaaggatttgtttgatccataaattgtgctaatgtgccacttgtaaaaaaacttttccatgctttactaacaacttttgcaaatactgacttattttgtcgccaaagaacctctaatgaactctgttcttctactttttttggctgttgtaatgcactcgtttttgggttaggtaatgttacaacttttctattaaaaaaagtctcaaattctctaattccacgtaacagctggtcttgtaagaattcatcacaacctcgaatacgtttttgtgttaaactatcaatttcatcaccaagcctttctttataaattaatttgagtaaagcctgtgttgctgctacaagatcttcagcagtaagagcatactggttaagtggttctccacttcccagtttttcttgaaattggtgccggccaatttttcctaataacaaaatctccttattccacaattttgtccaaaagaattcccgtgcaagagcatcagtgactaattcatctcgaatgtatgggttatattcaataaaatgagcgtaaagatagcgtaaagcttcttcttgagtctgtggatgaccatataaaccagctcggattagtattttgtcatgacgcgtttttccaacacttgtaatttcagaatcacgtttctgagaaaaatcttttacaaaactattacgaagaatatcagaatatttaaatcgttcaaaaagatctttttttgaaaaacctaatgcttgtaaaataacaagcaaagatacttttctccgtaaagttcgagtcgaaaaccataatcgatattttttatcaactgttaaattaatccaaccgcccttttctggaacaattcggacagttgatgcttgagttcgtgaatcccgaggaagtgtataaacaccaggattccgtaccatttgatgtaaaacaacacgaggaatgccattaattacaaaatggccatgttttgtcattaaaggtaaggatccgactaaaaaccattccaattttcgtgtcgatgagagactagatttaatactaacaggaatataaatagatgagctataagtttgtcctaaaacaaaagcttgtttttgtgtaaattcaggttgtctaaactgaactaattctggaaaaaattttatctcaagtgtttctgtaggtgtagaaaatgaaagagctttaggaaaagcttgtataagaccctgatttacaaaattaaaaaaactagttcgttgtatatctacaagatttggaatttcaaatggaaactgaaattgcgaagaagagagtgaaatgtcctttttttcacgttgttcttgcttttttgaaatttcgaagttagaaattaatctcgtttgagccgatgaacaacttacgatttttgaaaagtgagaataccccttgttagaattaataagccgatcatttgatcccaaaaacatcgtttttgggacaggtagtcttaaaaaatttgcttttagaccaaatttttcattttttggactggagaggaaatactttttttgatatttataacggcgaggaaaattcattttctaactgtattgtttttactgactttatgatattatctagagagaaagctccttttgacaaattatgaaacatttatttttgaaataaataggtgccaaaaggttcgaaagctttcgtccaatatttaaacggcggtatagccaagtggtaaggcagtggattgcaaatcctccatcccccagttcgaatctgggtgccgcctaataacatttcatttcagaaaaaatcaaacaaaaaatgactctaatttaaaaaaatttatggatgagacgtcttaaaaataaaaaaaatcttgagactttctttttttaaaaactcaactaattataataaatttgctaaattgttaagctcgcaaaaacaaattggttgtttttatatcgggaatctaacagtaaaaattttgattagaggtcaatctctttatgtttaaacagatttattagtttgagttttttaattaagttgattttctctgaaaaaaagaatgtaaaagtagtattacgtatttgaatttgtattataaaactttattattctgttttaatagagttttttattagcaaaaaaacgataaaacagaaaaaaaagggattcaccaaaatgtagtaaacctacttagtctctcatattattttcaacccttaatgctgcttcttttcagttctgacatgattttaagttgattaatacagagtgatgaatcccataaaattataatatcatacaataaaaatctttgttgattttaaattctaaatacaaaagtgattttagttccaaagaattcaaaattcaaagaatgttttaaatcaatttgtatctgacccaaatttaaataatttgcggctaaagtaatttaaatagattaaaaaataaatgatttttgtctggattgaataataatgcaattataacttctttttagaaaaaaaaattataaagaatgtattttaatatttaaaattaaaaaattttaaattaatacaattactacttgtaaaacttgcactagtatgttagtataaactctaatagacacactttctttccgcttaatggataaaacgttgtaaaaaactttttcaaaacttcttcatgaatgatatcgtctctagacttttttttggcgggttttacttatccacaaaaactcggtttataatcttcccccttcccaaaatttgagaaattttgggaagggggaaggtcaagagataaatttttaaaggttttttacaaccgaccattttctgattgatttgcagtaattttttcgggcagagtgcaagatcgtaaaccagagattttttattagaatggctccacaaactgaaactagagcaggtgctggatttaaagcgggtgttaaagactaccgtttaacttactacactcctgactaccaaccaaaagatactgatattcttgcagcattccgtatgactcctcaaccaggtgttccaccagaagaagctggtgcagcggtagcagcagaatcatcaactggtacttggacgactgtatggactgacggtttaactagtttagatcgttataaaggtcgttgttatgatatcgagccagttccaggtgaagaaaaccaatatattgcatatattgcatatcctttagatctttttgaagaaggttctgtaacaaacttattcacatctatcgtaggtaacgtatttggtttcaaagctcttcgtgctttacgtttagaagatcttcgtattccacctgcatatgttaaaacattccaaggtcctccacacggtattcaagtagaacgtgataaactgaacaaatatggtcgtggtttattaggttgtacaatcaaaccaaaattaggtctttcagctaaaaactacggtcgtgctgtatacgaatgtttacgtggtggtcttgacttcactaaagatgatgaaaacgtaaactctcaaccattcatgcgttggagagatcgtttcttatttgtagctgaagctatttacaaatctcaagcagaaactggtgaaattaaaggtcactatttaaacgctactgcagctactgctgaagaaatgttaaaacgtgctgaatgtgctaaagatttaggtgtacctattatcatgcacgactacttaactggtggtttcacagcaaacacaagtttagctcattactgccgtgataatggtcttcttttacacattcaccgtgcgatgcacgcggttattgaccgtcaaagaaaccatggtattcacttccgtgttttagcaaaagctcttcgtttatcaggtggtgaccacttacactcaggtactgttgtaggtaaattagaaggtgaacgtgaagtaacattaggtttcgttgacttaatgcgtgatgactacattgaaaaagatcgtagtcgtggtatttacttcactcaagactgggtttctttaccaggtacgatgccagtagcttctggtggtattcacgtatggcacatgccagctcttgttgaaattttcggtgatgatgcttgcttacaatttggtggtggtactttaggtcacccttggggtaacgctccaggtgctgctgcaaaccgtgtagctttagaagcatgtacacaagctcgtaacgaaggtcgtgaccttgctcgtgaaggcggtgatgtaatccgtgcagcgtgcaaatggagtcctgaattagctgctgcttgtgaagtttggaaagaaattaaatttgaattcgaaactatcgatacactttaatttcatttttcatacactcaaacttgttctttctgccttcctacatttttaagttgcttccaaaaactctgtttttgggacaggaaaaacagaaacgatcccttcccaaaaatttcatttttgggaaagagatttctcgaactaagtttgacgtcattctcacttttttaatcatttggattgcccccaattatactttgggggctacccaaatgtgtatctcattttgtaaattcatagaattgataatatagttcgtttttaaaacttaaaaaccctcaaaaaggtttcggagattgaaactctcggcaagaaaaaaacttttggaagtttaatttggtctcaaaaacgaaatttttggaattcaaacattttttatccccaaaccttttttcgggaagggcgcaattctgatttttgtgacagagaacagtagagaagaaactctaaaaaaataagaaactcctttaagatttaaacaaaacttcaaactgaaaacttaaatcttaaagcaaccatttaaattacacctaaaaaaattagtaaatatggcaaaaaaaagcatgatagagcgtgatcgtaaacgtgctcgtttaattacaaaatatgcttcaaaacgagaacaactccttttagaaatcaaacaagcttcttctctggaagaaaaattagcgttacatcgaaaattacaacaattacctcgtaacagtgctccagttcgagctcacaatcgttgttctattacgggtcgaccgcgagggtattttcgtgattttggcttatcacgacatgttttacgtgaatatgcacttcaagggttacttcctggcgttgtaaaagcaagttggtaatataaatgtttcttgactttttgggtttcaaattacccaaaaagtatgacttttatcttaatctcttttcaagaaactgtttttttgcaaaaaaaataaaaactcgtctcttccatttcctgttccaaaacaataattttaccacttctccaaactttttcaaaaaaaagcctgttttgaaaaggtttgtgatataaacttatttttaccccaaatttgaaaagttcctctcttcccaaaaatgcaattttttggataaacactgtttttggagatggaagtggtaaagggctaactctcaaactttttgagaaaacttactttctcaccaaaattttgcatccagttgggtttgaaccaacgatgtcctttcggagtcgcattatgagtgcgatgcaatcgaccgctctgccatggatgcttttatgtacagactaactaaagttatgtttcaaaacttacaaccagtagattttttaattcttttatttggttttctttatggaaatctttttgttataacattctcaaggtttcagtgggatttttttttaattttttctgttgtactttttttggagttactaaataaattgtgttatttaaattataagaaatacaaaagtctccacgagaagaaagctcaatacgagagtcaattttgtgaaaaagctgtaaaaatacataaaaaatcagttttctttttattccaacttttcaaaagaggttttcttttaggtttttttgttgaagcttttaaagtcggtagttaaattttgtaaaaaattatgatgattgaaaggatatcctttcaatcatcataatttttttaataagctaatcccatgcttcgagttgtttcagaacctaaatagacacgaacacttaagaaatctgtcggacaagcagattcacaacgtttacaaccaacacaatcttcagttcttggtgcagaagcgatttggtttgctttacagccatcccaaggtaccatttctaaaacgtctgttgggcatgcgcgaacacattgtgtacaaccaatgcatgtatcataaatttttactgtatgtgacatacttaaactcctgttttgggctagtcttgtttttctctactaaaaaaaattaatttgcaaaattaaaaaatttaatatttgaaattttttaatagataaatctcaagacactttcagtgataggttttataagattgaagatctcttcttgtttagtatatcgaaaaaaactaaaaaaaacaattttcttttaaaaaattttttaaaagaaaaaactaggatgaaaacctccccaggtaggacttgaacctacgaccaatcggttaacagccgaccgctctaccgctgagctactgaggattttttgtttaattaaattttatcaaaaaaaaagttttttgtaaaggagagtcttctctccctttcacaataatatcacttaatgggaaaaactttgtttttgagacaggctaaaaaaagctatctagttctagcattagacacaaatatttgtatctattcaactttcaaaaaaacatttaataaaacaaaattctgctttttaataaatttgtttttagaaaaagacaattaccgcgaagtgaaaccctttttgtctgtaataaaaaaaatgtctattaaatacaacatcccaacagtagcagagacaaaaatcacagagtatactaatactatcttctttgttttatttttttagtagtatgacagaaacgacacaatttgttagcccagaaaataattttggtcaagagcagacatcagaagccgtaaatttggtaactgagaaaaattctggttcggagagggtaattgtgattacgtctggaaaaggcggtgttggaaaaacaacaacaactgccaatttaggaatgtcaattgctcgtcttggatatcgggttgctttaattgatgcagatataggattacgaaacttagatttattattaggattagaaaatcgtgtactttacacggctatggatattgttgaaggacagtgtcgcttagatcaagcgttaattcgagataaaagatggaaaaatttagctttgttagctatttcaaaaacaaggcaaaaatataatgttactagaaaaaatatgcagaatttaataaattctgttaaagaacttggttttcaatttattttaatcgactgtcccgctggtattgatgttggttttgtaaatgcaattgcaccagctgaagaagctgttattgtaactacaccagaaattactgctattcgagatgccgatcgagttgctggtcttttagaagcaaatgggatttataacgtgaaattaattgtaaatcgagttcgacctgatatgattcaaaaaaatgacatgatgtctgttagagatgttcaagaaatgttagggattccattattaggtgctattccagaagatacaaatgtaattatatcaacaaataagggtgaacctcttgttttaaacaaaaaattaactctttctggtattgctttcgaaaacgcagctcgaagattaatcggaaaacaagattattttattgatttaacaagtcctcaaaaaggaatgtttcaaaaattgcaagaattttttcttggtgaggaataaattgatatgttttctttttctgctcttaaactcgacgttattaaaatttgttaagagcagaaaaagaaaacaaagttttttagaaaaccgaaaaacaaagtctcaaatagcatttctataaataatagaagaatttagcctctctcaaaaatttcatttttgaaaaatttggaatcaaactttgttaaatcttttttctaaaattgtgttattttacaaattttatttagccctcaaaaaaattaaatttaagggaaaacgaaaaaaattggttaccccaaatttttcaaatttggaaaagggaaagaaatcaaattaaaaatatttttctaaatatttgttttttattaaaatttttttcgggcgaggagggattcgaacccccgacaccgcggttcgtagccgcgtgctctagtccactgagctacaggccctagtaagtgaatattaaaagtatacactttataaaaaagtcttgcaataccaatttaatttttttctcttttatccaaacaaaatttggataaaagagaaaaaaaaaaatattaaaaaacttcaatttgtcttttttgatatcaaaacagtaaaatttaatttttgacgagttgattccaccctaaactttcaagtgcttgatttcgacgtaatggtcgagtaacaagttcaagaatatctcttgcatttgtaaacccatgaatttgtgcaaaagtaaattctacagaccattttgtattaataccacgagcttctaatgggtttgcatgagccataccagtaattgctaaatccggttggagttcacgtattcgttgaatttgattgtaattatctggtttttccactatgcgtggcatagagacttgcatttgtttacatgttttttctagaagatccaattcggctgcttgaaatcttttatctaaataggggattcctacttcataaacactcattccacaacgaattaaaaaacgtgctaaagatatttctaggaggttatctcccataaagaaaactgattttccacgaatcaaagaaatataatcttctaaagattcccaaatttgattttctctttcaacaagacctgaaggaaccacgccaaaaactgaacatattttttctaaccaggcacgagttccatctggaccaattggaaaaggagcactaatcaatttacattttcttcttcgcattaaggtagtagctgtacgactcaaaaaaggattaactccacagacgtaaacattttcattaagaactggtaaatcagcatatcgtggcgatggtagccagcctgcgacttgaattccacaacgttctaattctaaagttaattgtgtagcaacgtttgttggcaatgacccaaataaaactaaagatttttcagcagtttttgattccgtcccgttttcaaaacttttatttttcaggctaatgtggttcttgggagtatgacctttttctttttcgcttgtagaatttgttggacatctttgtaccattgaagctaatacagtgtcttctccttgtgtaaaagcataatctagtccatttgctcgtgcaacaacgataggagtttgaatttcagcttctaatcgtggagccatcccttctaagtccattttaataatttcagttgtacatgttccgatccaaacaataacactaggatttcgatcttgtttaatttgtaaacataatctttttaattctttataatcatttaattgagcagaaatgtcggcttcttctaattccgccatagcatagcgtggctcagcaaaaatcattacaccaagagcattttgaagaaaataaccacatgttttagttccaataactaaaaaaaaactgtcctctattttttgataaagccaagcaacacaactaatagggcaaaatgtatgataatttccagtttcacaatcaaaggttaatttttcagtactagataaatttaccatagttatatatatattaaatcaaacaaaaattaaataacccctttttacatgtctgccgatcaacatactaaccaattttctatggatttttttctcttccccaaattttaaaaatttggggaaaaatcaaggcaattttttatttaaaaagaaagaacctttgtaaaaaaattttaatttttattctaaaatcagtccaagttaaatgtttaaaaagatatgctaacttatttaggctgattttcctaagttttcttaacagactgaaaaaggagtagtaaaaaaagttcaagatttatgcttttatatttttttatttttttaaaccattaaaaaatctaattcacttgaactagtaattttttcatttgtaaaaatttgatcagttgttgactgtgaagttccagataaataataagttgataacaaagtaaataatttacggtcttcaagttcaacaggaattacaccttcagggtgtgctaaaagttgatcagcaatatttaaataaaaatcacaaatataagttaaacttgattcaaactctgccatttcaaatacagttttacctttgactcttgaaacacgaatatcttcaattaaaggtaaaacttcaagaacaggcattggacaaacttctacatatttatcaataagatcacgtttagcagttcgatttccaattagaccagcaagacgaagtgggtgagttttagatttttcccgaacagaagctacaattcggtttgcagcaaaaagagcatcaaagccattatcagtgactattagacaatagtcagcataatttaaaggtgccgcaaacccgccgcagactacatcacctaatacgtcaaaaagaataacgtcatattcataaaaagcatttaattcctttaaaagtttgactgtttcaccaacaacataaccaccacagcccgctccagctggtggaccacctgcttcaacagaatccacttccccatatccttgataaataacgtcttccaattgtgttaattttgataaaaaaaaattctatcaaaagaccctctaacacttagaataaagttcacacgttttgtataaatttttcggtgaaagtagacttagcaaaatttttagaccaagaagcgggttacaatacattttagagggttttactaaggcggaaatttctaatgtattgattaaataatgtactaaaaatgatagttttgggacaggtatttatagcttcaatttttacttttttcattatttctgaataactcacaaagagtttctttccctggtgtcataaaaaactaatcaatgaaaaaagtaaaccagattgttttgttagagggtctctttacatcgctgtaaagttcagactatatcatcaaccacagatactgtggttgtccggcgtgtagtcgttgaggggtcattaattgtttattaagaggacttccctgctgattgtccgcaccgagcatattatcaccatttattttttaggtttatttgttggtaatgtcggattctacaccacttttccttgttgtttggattgtggacgggtgttccagcatatagccagatttgaacttctctgttaccaggagaagtgccccaaaatgttaaggccagacgtcttcataatggtaatccttagcttgtaaggtatcaataatagttggaattaaaaaaccagttaaagtaaatgtactatcatgttttggatcacaaccaatctgtaatactttttttcctcgtcttgctaaagcaattgatatattacaacttgttgtagattttccaattccaccttttccataaaccgctaatttcataaaaaggaactccttttttagtaaacaatatctcttcaatttatttttattttgtgtttctcaaaaaattaataattttcatttgagcaataattaataaatggaataagttgattattttgatttgtcttttttattctatttcattttatttataaaaaaaattatatgttttgttttttactaaaataaaatttaatcggcataaaattaattagttaaaaaaatctatcactttaaaaaatcaattttttagttttcatttccctactagtcgtctataataaataaaaataaaatatgtgagattcttattttctgaattagtatctcagaagaaagcaattcttgaacttttttcttaaaaaaatagatttaaacttgattctttacatttgagtttaaaaattttgttttaatccaaagtattaaaaaaatctcaaaaaacaatctaacttaatccaaaaagtactagctttattttcctccgaactttattttttgatacctttcgttttatcccaaaaaccaagttttttagaaagggacggactcattttgtttttgagaaaaggggagagaccccagtgctttgatatgctaaaataaaaaacaagaagagcgttatagaaaatttttatggatattgcaaaaatcgaaagcttttgtattaatagctgctttattagtttatttattactacaatctattattggtgcaaaactctttttattccgaacccgacctatagtaaatcgggattagttggatatggaattgcatttatttttttaacaagccatttaatttttcgatggcttgattctggacatttccccttaagtaatttatacgaatccctcctttttctagcttggtgtttagtttttttgcagatttatctggaaatttttctgaaaactttatttttaggggtcttcacttcaccggctttgttatgtttaattgcatttacggacttaagtttacctcaagagttgcaaaaaagtgagcctcttgttccggcattacaatcaaattggttagtcatgcacgtaacagttatgattgcaagttatgcagctttattattaggttgtttactttcaattgtttatcttatttttttacaagtttttaaagtagaaacagaaaaaactacactttttgataattctcaaaataacggtgcttatcctattacaaaaaaatcttccaatctcaaccaaatttccgctttttccgaaaatttatctgactcttttcctttttcaaaaaacgaagttagtcccgaaaataaattttttgggcaagaggcatctattttggtagaaaaaaataaacaaatagaagccggtactatggcatttttctctgagaaagttgagaacacttcgtttctcacaatgttagataatctcagttatcggacaattggtattgggttttgtttcttgacattaggtattttgtcaggtgcgatttgggctaacgaaacttggggaaattattggagttgggacccaaaagaaacttgggctttcatcacttggttaacttttgcaacttatctacatagccgattaatagcaggctggacagggtcaaaacctgcttggattgctagctttggtttcattattgtttggatttgttatttgggtgtaaaccttattgggaaaggtcttcatagttatggttttttccaactttaaaaagtatttttctctttacgagagcttttttttcacgtatactaaattgtacgaaaatgggaaaaaatttagtctaatctacacttaatcccaaaacatggtagttcctatcccaaaaacatagtttttggggttaaatttcatttgctcccaaaaaacatagtttttcgggcaggaaaaggaattaataatttttttttcaattttatgtagaagttctctttgaaaaagagatagaaaacctataaaggagatatatatggcagttcctaaaaaacgaacatcaaaaatgaagacacgattacgtaaagctcaatggaaagctcaagctagtcgagaagcagcaaaagccctttctaaagcaaaaacaattcttaaatcattaagaagtgaatctgagggatcttcttcagcaaaaacagaagatgccccaacgcaaacaaactaatactgccccttggttacttttaacacgggtttcaaacggaaccacaccaatctttttaggattagcctaaagtttttatgaatactatattttccacgttgttaacaaaaattactctgtttgttaaaaaatttatcaatccgattctagttttaacaaacagagttggtccggccacaaaaactttgtttttgggattaaatagtgttagtcctaaaaatgaaagttttgggaaagggaaaggaaaaggtcaaaaaaaagccatttattccacttttgttttaaacatttttgttatttttcaagttttttttctgttaatcttacccctaaccgttttatttcttggaattgcacaaactgagtggagtgaagttataagaaaagcgactgatccaatcgctgtatcagcatacgttttaactgtgcaaatggctttatatgctgctttgctgaatactgtgtttggatttataattacttgggttttgacacgctaccaattttttggaaaaaagtttttagatgcagctgttgatcttccttttgctttaccaacttctgttgctgggttgactttagccacagtatatggagatcagggttggattggaagttttttcagtccttttgggcttcaaattgtttttacaaaaagtggtgttttactagctatgatttttgtgtctttcccttttgttataagaactttacagcctgttttgcaagaaatcgaacaaagtttagaagaagctgcctggtcattaggcgcatcaccgtggcaaactttccgtcgagttattttaccgacactttggccggcactttttacaggttttacgttaagtttttcacgagcacttggtgaatttggctcaattgttatgatttcatcaaacttaccatttaaagatttagtcgcttctgttttaatctatcagtcgttagaacaatatgattatttgggtgcatctgttattggttcagttgtgctgttaatagcttttacaaccttaattttaattaatgcgtttcaagcacttaaatatcgagtctaattgttgttttaaaaaaaaactcatgaagatccgaaaatcttcaaaataatttgaagattaaaacctctccatcccaaaaaacttttggaagtttaaccccaaaaacgatgtttttgggacatcctaaaaaaagtattttttttaggtgggaacagtcgagaaaaaattcgtctgtaacaaaaagaatctacagacgatcctacaaatcacaacatctgttttttaaaaatgaaatttatcccattcccaaaaagaatttttttgggaaccccatttactaagtaaatgaggataaacttcgtttttgagaaagggacagcgaaaaactgttcgttttgaaaattttgtaggaaagataagaatgtctttttgaaatgtttatagaaatagtagtttttaaatacaaaaccaatactttatgtcttttgtaaccgcaattcgagattatgttgaaactcttaatacagtaacaactacactgggtcaagattttacattgacaacttttctttctgaaacaggagtctatgttttaaaaacattgcaaagtgcgttactttatgttgtgagttttcagtggattagagattttactttattaccaatagttttacctcaactttctactgcccttttcaaagaaacttttgttttagaaactccagcaaaggtctttttcgattttttagaaataccggactttcatcaaaataaatttcttttaggtttttttaatagcttttttttcactttacctctttctattgttcacattgtcactgttcgaagacttttaattcaaggaattccatctgctgtttatacaattggtggttatttgcttgggcaaattttctttttaacatgtacagtttttggtcttcgatttattttgatcccttggcttacactcgaacctttgaattatgttctcggaattattttaatttttcgaactatttatcgaatggtcagagaaaatttagttgaattaaaaggctggggtgatgtaataacttggccacgctacaaatcattctttattacaagttttattttagcttggtgtgagcaaagttcaatttttctcttatttaggaaatctcacatttagttcaaatgtgagccttttagaaagtttttcaagcagtaatcctctttcgagtttttttactcatattacctacatcctagggatttctattggagctttgtttttcacttgtctttggggtattgttttcttacaagtcaaaaatttgtttttaatctatacaccactttttacaagtacatttattcaatggattaataaaacaacgtttgttgttactcttgctttaagtttaacttcgattccattttatggatttgaatatctcatgacaagccctttaggttttgtttcacaagacagtgtttttaaaacaactatttttgaccaaacccgtgtcaaagatattggtggaattggcttattgtcaagcccggaaagtaatcttcaatttcttgatgttgaagttgctccttttgatcgaggtgaatatcttactactcccggtgtagcacaaccattaagttttgaagagttaaattatcggggtgagtttgattggattgctcgacaagataaagtatctacccttactgattcaagaggtggtttttttagtctttcaaaactttttaaaaaacaattaaaatctcaagaccaaatagattctcgtcagcttgatcgaggtgatatcaacacattcctttctaaaaatgatcagactgctgcgtttgatctgttagacactagttctccagttattcaaagatttactacagaatcagctttagattttgagaattcgcaagaaaacgatcttataggttcatatcatgatctttatattacaagttttccagaaaatagagtgaatccacaaggagaaattgaaaaaaatatacaaaagaaaattaagcaaaaatattattcaaatcctgtctataaaacacttttaactttagacattgatttgtttttaaatcgccaacctaaaacatttcaattaaatgcttcacaagaaattgatttagatacaaaacgaaaaatggttgaatcttattatgattcattacgagaatacgctaaccttccttatagtgaaagttttgacgttttttttgatgggacaaagagtttttctaataaagtttataatcaacagtttaaaggaactttaagatcagttcgccgccttttctcactaacaccaaattctgaagaccaatccgtttcaacatctgaaaaatttagtggcaaaacagtcttaaaatttgatcaacctgcttatgagttgtctgaaaatcaaatgttctcggcatatcacgaagagttggttcctcaagaaaaacaagtgggaaaagataatcttctaactgacggattaactcaaaattttaaaaatgagaaaggaataagtttaataggaaatcctttgtatgctggttgggatgaaacaatgcgtaaatttgtcattacaaataaacttcttccaagaagttgggctggttataaagtaatttttcaaccagaaaatcaagctgttttagcacaaaaatcacaaaagcaaaaacagaaaattaaatttacagcatggccactttcacaagaacttattaacaatcaatcaacaattcactctccatatattacgctttttaagccacgctctattttgacagataaaggtcaacaacttacatatcaaatcccagcaaaccttgaacgttttgaattaaataagaaaaaagaatatcaatttatttctttacaaccaaaacgtggcggttttatttggccaggaaatgatcaatttaatttttctaccgtttttccgtttctgaaggctaactaagtaagttctaatgtttttcaattttttttctaaaaaacttttctttaatttcaaaaacatccttttttaaagagcaccaattatcaaaaaaaaccaagaggattaatcctcttggtttttgtacaatttctatattttccccttgcctgcctcaaaaacgaaattttcgggacaggcattcatcctaaaaaaagtctttttttaggcagccgtttaatgtaaaaaacaacgttttacccaaaaattttcaaagtttggggaggaaaaaaacttagtctttttaatttttaattaagttttaaaaaagacctaatgttaatgaaatatcaatagggagagcagcaccaattcctaaccaaattgctactactgtaccaattaaaaagacagttgttgcaacagggcggcggaaagggttttgaaatttgttgatattttcaataaatggaacagttaatagacctgctggaactgctgccattaaaagaacacctaaaagtttatttggaactacacgtaaaatttggaaaaccggataaaaataccattctggtaaaatttctaatggtgttgcaaaaggatttgctggttcaccaattgctgctggatctaaaactgataagccgataacgcaagcaaaagtaccaaaaattactactgggaaaatgtaaagtagatcatttggccatgctggttcaccataatagttatggcccatgccttttgcaagctttgcacgtaatactggatctgataaatctggttttttagtaactgccataaaagtctcctttgagaaaatctccccttttttttccaaaaacaccgtttccttccaaaaacttcgtttttgtttttggggcgaggactacacacgtttaaataattagaacgtttatgctttggaatgtttgaaaattagatattgaatgttttttgactgaaataccattcagtatcttgttgtgggagagttaaaacgggcattggggtctctttccaaaaattgataattttcgggttaactttgttttcccaaaaatactatttttgggactagctaggcttgtgaattggattgtttttgtttcaaataacagtaaaaattccaaaaaacacgttcattagttttttatttttcaaactaagacaaagaaagtcttggcgtaaagttgtaagagaaaattccagtttgcctttgttttctggtttttttaaagtggaccagaaataccttgtttacgaatcattaaaaagtgcatcagcataaagactgcagtcaatagcggtaacacgaaagtatgaagactatagaaacgtgttaatgttgcttggcctacaccaacaccacctcttaataattctactaaagctggtccaacaccaggaatagcgtcaggaacacctgttacaatttttaccgcccagtagcctacttggtcccatgggagtgaatagcctgttacaccgaaagaaacagtacatacagccatgataacaccagtaacccaagtaagttcacgtggctttttgaatccaccagttaaataaactcgacaaacatgaaggatcatcataagaaccatcatactagctgaccaacggtgaattgaacgaattaaccaaccaaagttcacttcagtcatgatgtactgtaccgaagcaaaagcttctgcaactgtaggacggtaataaaaagtcatagcaaaaccagttgctacttgaactaaaaaacatgtaaaagtgattccaccaatacaataaaaaatattaacatgtggtggaacatatttacttgaaatatcatcagcaatggcttgaatttcaagacgttcttcaaaccaatcgtacactttacccataaacaattttgaaaagatgtaaatacgcgtactttgttttttacataaaccctatttaggctaaaaaaaacgcaactacgcattaaaactagttagaccacaattaggctaaaataggcaaaaaattatcgtttttcttccattctcaaaaacaaattacggaagcgcttccttgccctaaaaacttcgtttttgggacagggaaagcgattaatattttttttaacccaaaacatacttactgggttatttttggagaaaggagcgagtaattttcgaattgtggatgattagtttgttgaccattttgtatcgacagctacttgatccacaagaccatagtcttttgcttcacgagcggataaaaactgatctcgatccatatccctcgaaattcgacttaatggttgacctgttctttcggcataaatgcgaccgacttgtcgtctaattcgcattacttcttcagattcagaaagaacttcggaagcttgcccttgacttccgccttcaggttggtgaatcataatacgagaatgtggaagagcaattcgttttccacgatcaccaccggctaaaataaaagaagccattgaagctgctgttcctacgcaaattgtagtcacgtctgatttaatatagttcattgcatcgtatacagcaataccacatgttacagatccaccaggagaattgatataaatatacagacctttattttgttcttctgcatttaaataaagcataataccaatgagttgatttgccaactcatcatctaattcttgacagagaaataaaacgcgttcccgatataaacggttatataaatcaacccattgagctgcaggttcaccgggtaaacgaaaagggactttagggacaccgattggcataatttacatgttgaatactttctaaattcttgttatttctttagtagttttttatttaaaatattataataagaaataacaagaaagacttttattaaaactaaagatctagatttagtatactctaaaagttcgaaaaattaaagaaaaaattgaaaagaataaatcccattaaaattacaatatcgttcttttttatctgctttaattttttatataagtaaaaatcttagtttgtagcatcttgaaaagatgctaggtaaaaaaaatgtgatatactaaaattaagaacacgtagtataaaaaagcagttatttttttctcatttttgactcggtttttgatgctcttctagatattggtaaatttttcaaattttttgttaaagcttattttatttttaatgagcttttctttatttaaggcatcagttaaattttacaaacaaaaaaacttctaaaattatttagaaagtcagtgctaaaaattagtttcaacctaaaaaggaagcttttgcgacacacaaagttcaatttttagttaaaaccgtcaattttcatataaaaacgacattttttttcgcagaatgagaaagaaataaaattagcctgttctaaaaaacaatgttttaataccaaaaatttcgtttatcttaaaatttgaaaaatttatcccaaaaacagtgtttttgggaatggacgggatagtattaaagtttgtttttggaagggcaaataaaacgcttttttatacgaaaagaaagaaagtaaaaaaaattacaaaagagagaattttttatgggtctcccatggtatagagttcatacagtagttttaaacgatccaggtcgtttaattgctgtgcatttaatgcatacttctttagtttctggttgggctggttcaatggctttttatgaacttgctgtttttgatccttctgatccagttttaaacccaatgtggcgtcaagggatgtttgttcttcctttcatgacacgtttaggcatcactcaatcttggggtggttggactattagcggtgaaacagcatcaaatccagggatttggagttatgaaggagttgctgctgctcacattattttatctggattactatttgcggcttctatttggcactgggtttattgggatcttgagcttttccgtgacccacgcacatcaaatccagcattagaccttccaaaaatttttggtattcacttatttttatcaggtcttctttgttttggtttcggagctttccacgtaactggattatttggtcctggtatttgggtttcagacccttacggtattacaggaactgttcaagcagttgctccttcttgggatgctacaggatttgatccttataacccgggtggcatttcagcacaccacattgctgcaggtatcttaggagttttagctggtttattccacctttgtgttcgtccaccacaaagattgtataacgggcttcgtatgggtaatattgaaactgttctttctagcagtattgcagcagttttctgggcagcgtttgttgtatctggaactatgtggtatggttctgccgcaacaccaattgaactttttggtccaactcgttaccaatgggatttaggtttcttccaacaagaaattgaacgtcgtgtacaaacaagtcttgctgaaggaaaatcagcttcacaagcttgggcagaaattccagaaaaattagctttttacgattacattggaaacaacccagcaaaaggtggtcttttccgtgccggtgctatgaatagtggtgatggtattgcagtaggttggttaggtcatgctgttttcaaagataaacaaggaaacgagctttttgttcgtcgtatgccaactttctttgaaacgttccctgtagtacttcttgacaaagacggtgttgttcgtgcagacgtacctttccgtcgttctgaatctaaatacagtattgaacaagtaggtgtttctgttacattctacggtggtgaattagatggtgtaacatttagtgatccagcaacagtgaaaaaatatgctcgacgcgctcaattaggagaaattttcgaatttgatcgtgcaactcttcaatctgatggggttttccgtacaagccctcgtggttggtttacttttgctcatttatgttttgcactccttttcttctttggtcatatttggcacggtgctcgtacaatcttccgagatgtatttgcaggtattgatgcagatttagacgaacaagtagaatttggtgccttcttaaaacttggcgatacttcaactcgtcgtcaatcagtataattccagttttctttaacttcctaaaaattctcaaaatttcaaaagtttctttttgaaatttttttacaagtctttagagaaaacagttaatctgctcctttccccatttcttaggaaatggggagaaacgcaggttttggaattaaaacgaagttagcctctcttgcaaaaacaaagtttttgagagagcggctaaccataaaagcaaataagagaaaaacggtaaatagtgcacttttctcccaaacttgatgatttttcgtctcgttggttactaacaaacagaacttttttctatggaagcgttagtttatacttttttattagttggaacattaggtattatcttttttgccattttctttagagaaccaccacgtattgtaaaataaaaataacagtttttccctcaaaggaaatttgagggaaaggttttcggtaagaaaaactttttttgtattttttttcttattccagaaattactaaattcggaataaaattttaaatttaaaaccatactcaagtgaaaactattactgttaaagactcccagtctaaaaaaatagttttcacaaaaaggaaaattaacagttccaa

(19) United States
(12) Patent Application Publication
(10) Pub. No.: US 2024/0368529 A1
(43) Pub. Date: Nov. 7, 2024

(54) A STRAIN OF CHLORELLA SOROKINIANA

(71) Applicant: ALIGA APS, Hjoerring (DK)

(72) Inventors: Sidsel Koggersbøl THRANE, Hjoerring (DK); Michael Krag NIELSEN, Hjoerring (DK)

(21) Appl. No.: 18/578,576

(22) PCT Filed: Jul. 11, 2022
(86) PCT No.: PCT/EP2022/069351

§ 371 Date: Jan. 11, 2024

(30) Foreign Application Priority Data
Jul. 12, 2021 (DK) PA 2021 70378
Dec. 17, 2021 (DK) PA 2021 70631

(51) Int. Cl. C12N 1/12; A01G 33/00; A23K 10/18; A23L 33/105; A61K 8/9722; C12N 9/50; C12N 15/01

(52) U.S. Cl. CPC C12N 1/125; A01G 33/00; A23K 10/18; A23L 33/105; A61K 8/9722; C12N 9/63; C12N 15/01; A61K 2800/10; C12N 2510/00

(57) ABSTRACT

The present invention relates to a strain and/or a modified strain of Chlorella sorokiniana (C. sorokiniana) having a reduced content of chlorophyll when compared to a wild type strain of C. sorokiniana cultured under the same conditions.

### TECHNICAL FIELD OF THE INVENTION

The present invention relates to a strain and/or a modified strain of Chlorella sorokiniana (C. sorokiniana) having a reduced content of chlorophyll when compared to a wild type strain of C. sorokiniana cultured under the same conditions. The present invention also relates to a method for producing the strain and/or the modified strain of C. sorokiniana, to a method for producing an algae biomass using said modified C. sorokiniana, to compositions comprising the modified C. sorokiniana and to various uses of the modified C. sorokiniana.

### BACKGROUND OF THE INVENTION

Algae are ubiquitous, photosynthetic organisms that appeared on the earth around 3.5 billion years ago. Since the taxonomic group of algae does not form a single monophyletic group, it is a very diverse group. They range from multicellular macroalgae, known as seaweed, that can reach a length of up to 60 m, to unicellular microalgae as small as 0.2 μm. Despite the common phototrophic abilities of microalgae, the group is heterogeneous. Industrial relevant species include prokaryotic species, such as the cyanobacterium Arthrospira with the common name Spirulina, as well as eukaryotic species, such as the astaxanthin-rich Haematococcus pluvialis, the halotolerant Dunaliella salina, and the green algae Chlorella, the first microalga to be isolated and cultivated in 1890.

Although microalgae are defined as photoautotrophic organisms, several species have the ability to grow heterotrophically. Heterotrophy is possible, when uptake systems for organic compounds are available, such as the inducible hexose/H+ transporter in Chlorella species. The ability to grow heterotrophically increases the value of the microalgae from a commercial point of view, since there are comparatively low productivities of phototrophic microalgal cultures, compared to other heterotrophic microbial cultures. Phototrophic cultures are limited by the inhomogeneous distribution of light through the culture, which results in zones with low or no phototrophic activities, a problem that is not present when cultivating heterotrophically. Heterotrophic microalgal cultures can be up to two orders of magnitude more productive than phototrophic microalgal cultures.

The world population is rapidly increasing and expected to reach 9.1 billion by 2050, which increases the demand for food. As the world population more than doubled from 1961 to 2007, the agriculture kept pace, and current projections estimate that this will not change. If the agriculture is to continue to keep pace along with the growing world population, it will have a huge impact on natural environments, since increased use of fertilizers, pesticides, and ground-water will be necessary, as well as the takeover of several hundreds of millions of hectares wildlands. Thus, alternatives to the traditional, environmentally destructive agriculture are needed. The cultivation of microalgae for human consumption can be part of a solution to this issue.

Microalgae are a good source of essential fatty acids, several bioactive compounds such as carotenoids, vitamins and minerals, and especially protein, including all the essential amino acids. Compared to other traditional sources of protein, species of Chlorella has a high protein content: Beef, fish, and chicken have a protein content of 17.4%, 19.2%-20.6%, and 19-24% (dry matter) respectively, soybean flour and whole eggs contain 36% and 47% protein (dry matter) respectively, whereas Chlorella sp. can contain 50-60% protein (dry matter).

The microalga now classified as Chlorella sorokiniana (C. sorokiniana) was first categorized as a thermotolerant Chlorella pyrenoidosa mutant, after being isolated by Sorokin in 1953, but genetic analysis has now classified C. sorokiniana as an individual species. On average, the C. sorokiniana cell is composed of 30-38% carbohydrates, 40% protein, and 18-22% lipids, measured in dry weight, and the strain C. sorokiniana UTEX 1230 is one of the most productive strains, when cultivated, that have been identified so far.

Microalgal biomass has an intense green color, a distinct taste, and odour. One reason for these distinct organoleptic properties is the high chlorophyll content in microalgae. Chlorophyll has an intense green color, which is difficult to mask, and since the color of a food product is the first parameter to be assessed, several green microalgal food products come with a low acceptance from the consumers. Additionally, a correlation has been shown between the content of chlorophyll and a grassy taste, which can also impact the consumer acceptance of microalgal food products.

Hence, it would be advantageous to provide a strain and/or a modified strain of C. sorokiniana having a reduced content of chlorophyll. In particular, such modified strain will accommodate the drawbacks of known strains and the applicability of such modified strain in various food and feed products will increase significantly.

### SUMMARY OF THE INVENTION

The present invention relates to a strain and/or a modified strain of Chlorella sorokiniana (C. sorokiniana) having a reduced content of chlorophyll when compared to a wild type strain of C. sorokiniana cultured under the same conditions. Due to the reduced content of chlorophyll the strain and/or the modified strain of C. sorokiniana has a pale color. Since the grassy taste and odour is linked to the content of chlorophyll the strain and/or the modified strain of C. sorokiniana is well suited in a variety of food and feed applications. Also, the strain and/or the modified strain of C. sorokiniana is well suited for production as it shows growth rates similar to the wild type when cultured under the same conditions.

Thus, an object of the present invention relates to the provision of a strain and/or a modified strain of C. sorokiniana as well as a method for producing the strain and/or the modified strain of C. sorokiniana. In particular it is also objects of the present invention to provide a method for producing an algae biomass using the modified C. sorokiniana, to provide compositions comprising the modified C. sorokiniana and to provide various uses of the modified C. sorokiniana.

Thus, an aspect of the present invention relates to a strain and/or a modified strain of Chlorella sorokiniana having a chlorophyll content lower than the chlorophyll content of a wild-type strain of Chlorella sorokiniana when cultured under the same conditions.

A further aspect of the present invention relates to a strain and/or a modified strain of C. sorokiniana comprising a content of chlorophyll a (a-chlorophyll), chlorophyll b (b-chlorophyll) and/or the sum of chlorophyll a (a-chlorophyll)+chlorophyll b (b-chlorophyll) at or below 11 mg/g dry cell weight.

Another aspect of the present invention relates to a method for producing a strain and/or a modified strain of Chlorella sorokiniana having a chlorophyll content lower than the chlorophyll content of a non-modified wild-type strain of Chlorella sorokiniana, wherein said method comprises the steps of:
a) obtaining a parental strain of C. sorokiniana,
b) subjecting the parental strain of C. sorokiniana to mutagenesis,
c) cultivating the mutated strain of C. sorokiniana on a medium comprising nicotine, norflurazon and/or diphenylamine and
d) identifying colonies of the mutated strain of C. sorokiniana having a phenotype different from the parental strain of C. sorokiniana as the strain and/or the modified strain of C. sorokiniana,
e) obtaining the strain and/or the modified strain of Chlorella sorokiniana, and
f) verifying and comparing the sequence of the chloroplast genome from wild type and the modified C. sorokiniana

A further aspect of the present invention relates to a Chlorella sorokiniana obtainable by the method of the present invention.

An even further aspect of the present invention relates to a method for producing an algae biomass, said method comprising the steps of:
(a) culturing the strain and/or the modified strain of Chlorella sorokiniana of the present invention and/or a composition of the present invention aerobic heterotrophically in a media and
(b) obtaining an algae biomass.

Yet another aspect of the present invention relates to an algae biomass obtainable by the method of the present invention.

An even further aspect of the present invention relates to the use of the modified Chlorella sorokiniana of the present invention or the composition of the present invention for the manufacture of an algae biomass.

A still further aspect of the present invention relates to a composition comprising the strain and/or the modified strain of Chlorella sorokiniana of the present invention or an algae biomass comprising the strain and/or the modified strain of Chlorella sorokiniana.

Yet another aspect of the present invention relates to a method for using the composition above as an ingredient in at least one of the group consisting of human foods, human nutraceutical preparations or formulations, animal feeds, pharmaceutical compositions including drugs, vaccines, cosmetics, personal care compositions, personal care devices or textiles, dyes, inks, packaging material and combinations thereof.

A further aspect relates to the use of a strain and/or a modified strain of Chlorella sorokiniana as defined above or an algae biomass comprising the strain and/or the modified strain of Chlorella sorokiniana as defined above in at least one of the group consisting of in human foods, nutraceutical preparations, nutritional supplements or formulations, animal feeds, pharmaceutical compositions including drugs, vaccines, cosmetics, personal care compositions, personal care devices or textiles, dyes, inks, packaging material and combinations thereof.

A still further aspect relates to the use of a strain and/or a modified strain of Chlorella sorokiniana as defined above, an algae biomass as defined above or a composition comprising the strain and/or the modified strain of Chlorella sorokiniana as defined above or an algae biomass as defined above for increasing the protein content in a food product and/or a feed product.

### BRIEF DESCRIPTION OF THE FIGURES

FIG. 1 summarizes the growth of the wild type (dark black) and M12 (light black) with regards to the dry weight (g/l) of the cultures. The dotted line represents a longer period in which it was not possible to take samples. Addition of nutrients to the culture is marked with a square.

FIG. 2—illustrates a HPLC chromatogram showing two isomers of chlorophyll-a, where the peaks are the areas of the pigment isomers.

FIG. 3 summarizes the growth of the wild type (C. sorokiniana UTEX 1230) on solid photoautotrophic medium and solid heterotrophic medium.

FIG. 4 summarizes the growth of the modified strain of C. sorokiniana (M12) on solid photoautotrophic medium and solid heterotrophic medium.

M12 is the strain and/or the modified strain of C. sorokiniana as obtained in Example 1-5.

### DETAILED DESCRIPTION OF THE INVENTION

### Definitions

Prior to outlining the present invention in more details, a set of terms and conventions is first defined:

The term “genus” means genus as defined on the website www.ncbi.nlm.nih.gov/taxonomy. An algae “strain” as used herein refers to an algae which remains genetically unchanged when grown or multiplied. A multiplicity of identical algae are included.

Chlorella sorokiniana (C. sorokiniana) designates a species of freshwater green microalga in the Division Chlorophyta and of the Genus “Chlorella”. C. sorokiniana can grow both phototrophically and heterotrophically. Phototrophical growth is to be understood as the formation of cellular carbon solely from carbon dioxide by photosynthesis while heterotrophical growth is the formation of cellular carbon using organic substrates as a source of carbon and in the presence of oxygen. Heterotrophical growth does not need light like phototrophical growth.

The wild type strain of Chlorella sorokiniana is to be understood as UTEX1230

“Chlorophyll” means any of several related green pigments found in the chloroplasts of algae. Two types of chlorophyll exist in the photosystems of algae: chlorophyll a and b. If not otherwise specified the term “chlorophyll” is to be understood as a sum of chlorophyll a and b. Besides being a green pigment chlorophyll also has a distinct “grassy” taste and a characteristic odour.

Carotenoids are pigments that exist in plants and algae, and which produce the bright yellow, red, and orange colors in them. There are around 750 naturally occurring carotenoids which for humans are important as they act as antioxidants as well as they can be converted into essential vitamins. Following carotenoids existing in Chlorella sorokiniana (C. sorokiniana)

“β-carotene” means a red-orange pigment that can convert into Vitamin A and act as an antioxidant.

“Lutein” means a red pigment with antioxidant effects that is important for maintaining eye health, reducing the risk of macular degeneration and cataracts as well as having protective effect on skin and cardiovascular system.

“Neoxanthin” means a carotenoid and xanthophyll that acts as a precursor of the plant hormone abscisic acid.

“Unidentified carotenoids” means Carotenoids that are present in the Chlorella sorokiniana (C. sorokiniana) yet haven't been determined by the standard curve measurement analysis.

“Fresh biomass” is to be understood as algae biomass used or analysed immediately after the end of cultivation, or algae biomass frozen immediately after the end of cultivation and thawed immediately before being applied or analysed.

“After storage” or “an algae biomass after storage” is to be understood as storage of an algae biomass for 1-14 days and at a temperature in the range of 1-32° C. before the algae biomass being applied or analysed. Storage can either be in daylight or in the dark.

In the present context, the term “mutant” or “mutant strain” should be understood as a strain derived, or a strain which can be derived, from a strain of the invention (or the mother strain) by means of e.g. genetic engineering, radiation and/or chemical treatment. It is preferred that the mutant is a functionally equivalent mutant, e.g. a mutant that has substantially the same, or improved, properties (e.g. regarding color, taste and/or odour) as the strain from which it is derived. Such a mutant is a part of the present invention. Especially, the term “mutant” refers to a strain obtained by subjecting a strain of the invention to any conventionally used mutagenization treatment including treatment with a chemical mutagen such as ethyl methanesulfonate (EMS) or N-methyl-N′-nitro-N-nitroguanidine (MNNG), or a physical mutagen such as UV light, gamma-rays, x-rays, or to a spontaneously occurring mutant. A mutant may have been subjected to one or several mutagenization treatments (a single treatment should be understood as one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the algae genome have been shifted with another nucleotide, or deleted, compared to the mother strain. As will be clear to the skilled person, mutants of the present invention can also be mother strains.

In the present context, the term “variant” or “variant strain” should be understood as a strain which is functionally equivalent to a strain of the invention, e.g. having substantially the same, or improved, properties or characteristics e.g. colour, taste and/or odour. Such variants, which may be identified using appropriate screening techniques, are a part of the present invention.

In the present description and claims the conventional one-letter code for nucleotides is used following the analogous principles as described for amino acids nomenclature supra.

Algorithms for aligning sequences and determining the degree of sequence identity between them are well known in the art. For the purpose of the present invention a process may be carried out for aligning nucleotide sequences using blast as provided by the National Center for Biotechnology Information (NCBI) on https://blast.ncbi.nlm.nih.gov applying standard parameter.

In the context of the present invention, a mutation in the gene (gene mutation) or modification of the gene is to be understood as an alteration in the nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift. In the context of the present invention, a deletion is to be understood as a genetic mutation resulting in the removal of one or more nucleotides of a nucleotide sequence of the genome of an organism; a insertion is to be understood as the addition of one or more nucleotides to the nucleotide sequence; a substitution is to be understood as a genetic mutation where a nucleotide of a nucleotide sequence is substituted by another nucleotide; a frameshift is to be understood as a genetic mutation caused by a insertion or deletion of a number of nucleotides in a nucleotide sequence that is not divisible by three, therefore changing the reading frame and resulting in a completely different translation from the original reading frame; an introduction of a stop codon is to be understood as a point mutation in the DNA sequence resulting in a premature stop codon; an inhibition of substrate binding of the encoded protein is to be understood as any mutation in the nucleotide sequence that leads to a change in the protein sequence responsible for preventing binding of a substrate to its catalytic site of the protein. Furthermore, a knockout mutant is to be understood as genetic mutation resulting in the removal or deletion of a gene, such as an entire gene or an entire open reading frame from the genome of an organism.

A “modified algae” or a “modified strain” as used herein refers to a natural (spontaneous, naturally occurring) modified algae or an induced modified algae comprising one or more mutations in its genome (DNA) which are absent in the wild type DNA. An “induced mutant” is an algae where the mutation was induced by human treatment, such as treatment with chemical mutagens, UV- or gamma radiation etc. In contrast, a “spontaneous mutant” or “naturally occurring mutant” has not been mutagenized by man. Modified algae are herein, non-GMO (non-genetically modified organism), i.e. not modified by recombinant DNA technology.

“Chloroplast genome” is to be understood as a circular DNA containing some number of genes necessary for functioning of the chloroplasts and maintaining their structure. These DNA also contain some genes of ribosomal and transport RNA. SEQ ID NO 1 is circular DNA, such that residue 109756 is adjacent to residue 1.

PSII reaction center protein M encoded by psbM is a protein located in the core complex of photosystem II (PSII).

t-RNA encoded by trnS-UAG is transfer RNA which transfer amino acids to ribosomes during translation of messenger RNA (mRNA).

PSII reaction center protein I encoded by psbI is a protein located in the core complex of photosystem II (PSII), and is required for the stability and/or assembly of PSII.

PSI reaction center protein Ycf3 encoded by ycf3 is a protein which is essential for the assembly of the photosystem I (PSI) complex, in which it might act as a chaperone in the assembly of the PSI subunits.

Chloroplastic ATP synthase subunit c encoded by atpH is a protein which act as a key component in the F0 channel in the ATP-synthase, an enzyme that produces ATP from ADP and inorganic phosphate using a proton gradient.

PSI P700 chlorophyll a apoprotein A2 encoded by psaB is an apoprotein that binds P700, which is the primary electron donor in photosystem I (PSI).

ATP-dependent Clp protease proteolytic subunit encoded by clpP is a protein-subunit in the enzyme Clp protease, which cleaves peptides in various proteins; a process that requires the hydrolysis of ATP.

The inventors of the present invention surprisingly succeeded in developing a strain and/or a modified strain of C. sorokiniana having a stable phenotypic color different from the wild type. The strain and/or the modified strain of C. sorokiniana showed growth rates comparable to the wild type, thus making it highly suitable for industrial scale production.

Thus, one aspect of the present invention relates to a strain and/or a modified strain of C. sorokiniana having a chlorophyll content lower than the chlorophyll content of a wild-type strain of C. sorokiniana when cultured under the same conditions.

In the present context “cultured under the same conditions” is to be understood as cultivation:
at the same temperature, pH and oxygen level
for the same period of time,
without the presence of light, and
in the same culture media.

An example of how to compare the strain and/or the modified strain of C. sorokiniana and the wild type strain could be done as follows: Biomass from the strain and/or the modified strain of C. sorokinana and the wild type strain C. sorokinana are inoculated into separate baffled 250 ml Erlenmeyer flasks with filtered lids containing 50 ml liquid medium. The cultures are incubated at 32° C. and 150 rpm for 3 days in the dark. The cultures are transferred to separate 1000 ml baffled Erlenmeyer flasks with filtered lids containing 100 ml liquid medium. The cultures are incubated at 32° C. and 150 rpm for 4 days in the dark. The cultures are transferred to separate 3-L bioreactors containing 1.8 l liquid medium. The cultures are cultivated in the bioreactors up to 30 hours in the dark, during this time nutrients are added, pH is automatically adjusted around 7, DO (dissolved oxygen) is automatically adjusted to 70-75%. During cultivation in the bioreactors, the dry weight of the cultures are monitored.

Another aspect of the present invention relates to a strain and/or a modified strain of C. sorokiniana comprising a content of chlorophyll at or below 11 mg/g dry cell weight.

In a further embodiment chlorophyll is chlorophyll a (a-chlorophyll) and/or chlorophyll b (b-chlorophyll).

A further aspect of the present invention relates to a strain and/or a modified strain of C. sorokiniana comprising a content of chlorophyll a (a-chlorophyll), chlorophyll b (b-chlorophyll) and/or the sum of chlorophyll a (a-chlorophyll)+chlorophyll b (b-chlorophyll) at or below 11 mg/g dry cell weight.

It will be appreciated that the strain and/or the modified strain of C. sorokiniana comprises a content of chlorophyll at or below 11 mg/g, such as below 10 mg/g, e.g. below 9 mg/g, such as below 8 mg/g, e.g. below 7 mg/g, such as below 6 mg/g, e.g. below 5 mg/g, such as below 4 mg/g, e.g. below 3 mg/g, such as below 2 mg/g, e.g. below 1 mg/g, such as below 0.9 mg/g, e.g. below 0.8 mg/g, such as below 0.7 mg/g, e.g. below 0.6 mg/g, such as below 0.5 mg/g, e.g. below 0.4 mg/g, such as below 0.3 mg/g, e.g. below 0.2 mg/g, such as below 0.1 mg/g, e.g. in the range of 0-11 mg/g, such as in the range of 0.1-10 mg/g, e.g. in the range of 0.2-9 mg/g, such as in the range of 0.3-8 mg/g e.g. in the range of 0.4-7 mg/g, such as in the range of 0.5-6 mg/g, e.g. in the range of 0.6-5 mg/g, such as in the range of 0.7-4, e.g. in the range of 0.8-3 mg/g, such as in the range of 0.9-2, e.g. in the range of 1-11 mg/g and preferably below 11 mg/g dry cell weight.

In the present context the amount of chlorophyll a and b is measured by solvent extraction followed by HPLC analysis using a photodiode array detector and as disclosed in further detail in Example 5.

It will be appreciated that the methods applied to measure chlorophyll a (a-chlorophyll), chlorophyll b and carotenoids like, Lutein, β-carotene, Neoxanthin and unidentified carotenoids all have a minimum detection rate. If the measurement is below the detection rate it will include complete absence of the respective compound.

In one embodiment the strain and/or the modified strain of C. sorokiniana comprises a chlorophyll a content at or below 7.5 mg/g, e.g. below 7 mg/g, such as below 6 mg/g, e.g. below 5 mg/g, such as below 4 mg/g, e.g. below 3 mg/g, such as below 2 mg/g, e.g. below 1 mg/g, such as below 0.9 mg/g, e.g. below 0.8 mg/g, such as below 0.7 mg/g, e.g. below 0.6 mg/g, such as below 0.5 mg/g, e.g. below 0.4 mg/g, such as below 0.3 mg/g, e.g. below 0.2 mg/g, such as below 0.1 mg/g, e.g. in the range of 0-7.5 mg/g, such as in the range of 0.1-7 mg/g, e.g. in the range of 0.2-06 mg/g, such as in the range of 0.3-5 mg/g e.g. in the range of 0.4-4 mg/g, such as in the range of 0.5-3 mg/g, e.g. in the range of 0.6-2 mg/g such as in the range of 0.7-1 mg/g, e.g. in the range of 0.8-0.9 mg/g-0.7 mg/g such as in the range of 0.1-0.8 mg/g, e.g. in the range of 0.2-0.7 mg/g and preferably below 7.5 mg/g dry cell weight.

In another embodiment the strain and/or the modified strain of C. sorokiniana comprises a chlorophyll b content at or below 4.5 mg/g, e.g. below 4 mg/g, such as below 3 mg/g, e.g. below 2 mg/g, such as below 1 mg/g, e.g. below 0.9 mg/g, such as below 0.8 mg/g, e.g. below 0.7 mg/g dry cell weight, e.g. below 0.6 mg/g, such as below 0.5 mg/g, e.g. below 0.4 mg/g, such as below 0.3 mg/g, e.g. below 0.2 mg/g, such as below 0.1 mg/g, e.g. in the range of 0-4.5 mg/g, such as in the range of 0.1-4 mg/g, e.g. in the range of 0.2-3 mg/g, such as in the range of 0.3-2 mg/g, e.g. in the range of 0.4-1 mg/g, such as in the range of 0.5-0.9 mg/g, e.g. in the range of 0.4-0.8 mg/g, such as in the range of 0.5-0.7 mg/g and preferably below 4.5 mg/g dry cell weight.

It might be an aim to use protein from algae and e.g. C. sorokiniana as a substitute or partly substitute from proteins derived from animals. The protein content of a wild type C. sorokiniana is in the range of 23-63% w/w and it may therefore be contemplated that the strain and/or the modified strain of C. sorokiniana comprises a protein content roughly similar to the wild type. Often protein content and yield are opposed factors when culturing algae and thus, optimizing yield often leads to a decrease in the protein content while optimizing the protein content often leads to a reduced yield. High yield leads to a high dry matter content.

It may be contemplated that the strain and/or the modified strain of C. sorokiniana comprises a protein content of at least 20% w/w, e.g. at least 25% w/w, such as at least 30% w/w, e.g. at least 35% w/w, such as at least 40% w/w, e.g. at least 45% w/w, such as at least 55% w/w, e.g. at least 60% w/w, such as at least 65% w/w dry cell weight, e.g. in the range of 20-65% w/w, such as in the range of 25-60% w/w, e.g. in the range of 30-50% w/w, such as in the range of 35-45% w/w, e.g. in the range of 40-65% w/w and preferably in the range of 12-15% w/w dry cell weight.

In the present context the amount of protein is measured by the Kjeldahl method (§ 64 LFGB L 17.00-15:2013-08 (mod.) on original washed sample g/100 g with parameter method Protein (N×6.25)).

Besides a reduced content of chlorophyll, the modified stain of C. sorokiniana according to the present invention uniquely also comprises a reduced content of other carotenoids (when compared to the wild type and when grown under the same conditions) like lutein, β-carotene, Neoxanthin as well as other unidentified carotenoids. If one pigment is reduced one would expect that the content of other pigments remains unchanged thus, obtaining a modified stain of C. sorokiniana having a reduced content of chlorophyll, lutein, β-carotene and Neoxanthin is extremely surprising and exceptional and increases the industrial applicability significantly.

Odour is linked to chlorophyll a and b and may possibly also be linked to carotenoids.

The strain and/or the modified strain of C. sorokiniana may comprises a lutein content below the lutein content of a non-modified wild-type C. sorokiniana when cultured under the same conditions. In a specific embodiment the strain and/or the modified strain of C. sorokiniana comprises below 1.5 lutein mg/g dry cell weight, such as below 1, e.g. below 0.4, such as below 0.9, e.g. below 0.8, such as below 0.7, e.g. below 0.6, such as below 0.5, e.g. below 0.4, such as below 0.3, e.g. below 0.2, such as below 0.2, e.g. below 0.1, such as in the range from 0-1.5, e.g. in the range from 0.1-1, such as in the range from 0.2-0.9, e.g. in the range from 0.3-0.8, such as in the range from 0.4-0.7, e.g. in the range from 0.5-0.6, e.g. in the range from 0.1-0.5, such as in the range from 0.2-0.4, e.g. in the range from 0.3-0.6 lutein mg/g dry cell weight and preferably below 1.5 lutein mg/g dry cell weight.

In the present context the amount of lutein is measured by solvent extraction followed by HPLC analysis using a photodiode array detector and as disclosed in further detail in Example 5.

The strain and/or the modified strain of C. sorokiniana may comprise a β-carotene content below the lutein content of a non-modified wild-type C. sorokiniana when cultured under the same conditions. In s specific embodiment the strain and/or the modified strain of C. sorokiniana comprises below 0.33 β-carotene mg/g dry cell weight, such as below 0.3, e.g. below 0.2, such as below 0.1, such as in the range from 0-0.33, e.g. in the range from 0.1-0.3, such as in the range from 0.1-0.3, β-carotene mg/g dry cell weight and preferably below 0.33 β-carotene mg/g dry cell weight.

In the present context the amount of β-carotene is measured by solvent extraction followed by HPLC analysis using a photodiode array detector and as disclosed in further detail in Example 5.

The strain and/or the modified strain of C. sorokiniana may comprise a Neoxanthin content below the lutein content of a non-modified wild-type C. sorokiniana when cultured under the same conditions. In s specific embodiment the strain and/or the modified strain of C. sorokiniana comprises below 0.16 Neoxanthin mg/g dry cell weight, e.g. below 0.13, such as below 0.10, e.g. below 0.7, such as below 0.4, e.g. below 0.1, such as in the range from 0-0.16, e.g. in the range from 0.1-0.13, such as in the range from 0.4-0.13, e.g. in the range from 0.7-0.10 Neoxanthin mg/g dry cell weight and preferably below 0.33 Neoxanthin mg/g dry cell weight.

In the present context the amount of neoxanthin is measured by by solvent extraction followed by HPLC analysis using a photodiode array detector and as disclosed in further detail in Example 5.

The pigment analysis and protein analysis may be performed on fresh algae biomass or an algae biomass after storage.

In an embodiment of the present invention The strain and/or the modified strain of C. sorokiniana comprises at least one mutation in, upstreams or downstreams of one of the genes comprised in the chloroplast genome.

Genes of the chloroplast genome may be selected from the group consisting of psbN, psbH, trnY-GUA, psbM, trnS-UGA, trnG-GCC, trnM-CAU, trnE-UUC, rpl20, rps18, trnW-CCA, trnP-ugg, psaJ, rps12, rps7, tufA, rpl19, ycf4, cemA, rpl23, rpl2, rps19, rps3, rpl16, rpl14, rpl5, rps8, infA, rpl36, rps11, rpoA, rps9, rpl12, trnR-UCU, chII, petA, petL, petG, psbD, psbC, trnS-GCU, psbI, ycf3, trnR-CCG, trnT-UGU, rps2, atpI, atpH, atpF, atpA, trnM-CAU, trnG-GCC, trnD-GUC, atpB, atpE, rps4, trnL-GAG, trnS-GGA, rrnS, trnI-GAU, trnA-UGC, rrnL, rrnF, trnL-UAG, trnV-UAC, psaI, accD, cysA, trnT-GGU, ftsH, psbE, psbF, psbL, psbJ, trnL-CAA, trnQ-UUG, psaM, ycf12, psbK, trnG-UCC, trnH-GUG, ycf47, trnF-GAA, trnK-UUU, psbZ, chiB, psaA, psaB, psbA, rpoC2, rpoC1, rpoB, trnC-GCA, rbcL, rps14, trnM-CAU, psaC, trnN-GUU, minD, trnR-ACG, chIN, chIL, ccsA, rpl32, cysT, ycf1, petD, petB, clpP, psbB and psbT.

Upstream of a gene is towards the 5′ end from the transcription initiation site of the coding sequence.

Downstream of a gene is towards the 3′ end from the transcription initiation site of the coding sequence.

In one embodiment the one or more mutations are located in, upstreams or downstreams of a gene associated with:
Photosystem I and photosystem II
Chloroplast ATP synthase,
Chloroplast Clp protease and/or
Transfer RNA

In order to obtain the reduced content of chlorophyll it may be contemplated that the one or more mutations are located in, upstreams or downstreams of a gene associated with:
Photosystem I and photosystem II, such as
   psbM
   psbI
   ycf3, and/or
   psaB
Chloroplast ATP synthase
   atpH
Chloroplast Clp protease
   clpP
Transfer RNA
   trnS-UAG

In an embodiment of the present invention one or more mutation(s) are located in the promoter region of any one of the genes comprised in the chloroplast genome. When a mutation is present in the promoter region it means that the gene itself is not affected; it is the regulation of the gene-expression that is affected.

In a specific embodiment the one of the genes comprised in the chloroplast genome is selected from the group consisting of:
psbM encoding the protein PSII reaction center protein M
trnS-UAG encoding t-RNA
psbI encoding the protein PSII reaction center protein I
ycf3 encoding PSI reaction center protein Ycf3
atpH encoding ATP synthase subunit c
psaB encoding PSI P700 chlorophyll a apoprotein A2
clpP encoding ATP-dependent Clp protease proteolytic subunit
and/or combinations thereof.

In an embodiment the one or more mutations(s) are located in
(a) atpH.

In an embodiment the one or more mutations(s) are located upstream of:
(a) psbM,
(b) psbI,
(c) ycf3,
(d) cipP

In an embodiment the one or more mutations(s) are located downstream of:
(a) trnS-UAG, and/or
(b) psaB

In a more specific embodiment the one or more mutation(s) are:
located in atpH,
located upstream of a gene selected from the group consisting of psbM, psbI, ycf3, clpP and any combination thereof, and/or
located downstream of trnS-UAG and/or psaB

In a more specific embodiment the one or more mutation(s) are:
located in atpH,
located upstream of a gene selected from the group consisting of psbI, ycf3, clpP and any combination thereof, and/or
located downstream of trnS-UAG and/or psaB

In a more specific embodiment the one or more mutation(s) are:
located in atpH,
located upstream of a gene selected from the group consisting of psbM, psbI, ycf3, clpP and any combination thereof, and/or
located downstream of psaB

In an embodiment the one or more mutations(s) are located in
(a) atpH at a position corresponding to position 34544-34554 in SEQ ID NO 1

In an embodiment the one or more mutations(s) are located upstream of:
(a) psbM and at a position according to 2992 in SEQ ID NO 1,
(b) psbI and at a position according to 29466 in SEQ ID NO 1,
(c) ycf3 and at a position according to 30075-30076 in SEQ ID NO 1, and/or
(d) clpP and at a position according to 107103 in SEQ ID NO 1

In an embodiment the one or more mutations(s) are located downstream of:
a) trnS-UAG and at a position according to 2992 in SEQ ID NO 1, and/or
b) psaB and at a position according to 72653 in SEQ ID NO 1

In a specific embodiment the one or more mutation(s) are:
located in atpH at a position corresponding to position 34544-34554 in SEQ ID NO 1
located upstream of psbM and at a position according to 2992 in SEQ ID NO 1, psbI and at a position according to 29466 in SEQ ID NO 1, ycf3 and at a position according to 30075-30076 in SEQ ID NO 1, and clpP and at a position according to 107103 in SEQ ID NO 1, and
are located downstream of trnS-UAG and at a position according to 2992 in SEQ ID NO 1 and psaB and at a position according to 72653 in SEQ ID NO 1.

In a specific embodiment the one or more mutation(s) are:
located in atpH at a position corresponding to position 34544-34554 in SEQ ID NO 1
located upstream of psbI and at a position according to 29466 in SEQ ID NO 1, ycf3 and at a position according to 30075-30076 in SEQ ID NO 1, and clpP and at a position according to 107103 in SEQ ID NO 1, and
are located downstream of trnS-UAG and at a position according to 2992 in SEQ ID NO 1 and psaB and at a position according to 72653 in SEQ ID NO 1.

In a specific embodiment the one or more mutation(s) are:
located in atpH at a position corresponding to position 34544-34554 in SEQ ID NO 1
located upstream of psbM and at a position according to 2992 in SEQ ID NO 1, psbI and at a position according to 29466 in SEQ ID NO 1, ycf3 and at a position according to 30075-30076 in SEQ ID NO 1, and clpP and at a position according to 107103 in SEQ ID NO 1, and
are located downstream of psaB and at a position according to 72653 in SEQ ID NO 1.

It may be contemplated that at least one mutation(s) is/are point mutations. It is to be understood that a point mutation is a mutation where a single nucleotide base is substituted, inserted or deleted from the DNA.

In a specific embodiment the mutation(s) on the nucleotide level are:
(a) a substitution from G to A at a position corresponding to position 2992 in SEQ ID NO 1;
(b) a substitution from T to A at a position corresponding to position 29466 in SEQ ID NO 1;
(c) a deletion of AA at a position corresponding to position 30075-30076 in SEQ ID NO 1;
(d) a deletion of CAGAAGCAGAA at a position corresponding to position 34544-34554
(e) a deletion of A at a position corresponding to position 72653 in SEQ ID NO 1, and/or
(f) a deletion of A at a position corresponding to position 107103.

We have determined that the C. sorokiniana M12 strain (deposit number CCAP 211/136) has the six mutations (a) to (f) immediately above, which strain was sequenced as described in Examples 4 and 5 below.

In a further embodiment, the invention provides a C. sorokiniana strain having the mutations of the C. sorokiniana M12 strain (deposit number CCAP 211/136).

In a further embodiment, the invention provides the C. sorokiniana strain according to deposit number CCAP 211/136.

In one embodiment the chloroplast genome comprises a nucleotide sequence having at least 98% sequence identity to the sequence of SEQ ID NO 1, such as at least 98.1%, e.g. at least 98.2%, such as at least 98.3%, e.g. at least 98.4%, such as at least 98.5%, e.g. at least 98.6%, such as at least 98.7%, e.g. at least 98.9%, such as at least 99%, e.g. at least 99.1%, e.g. at least 99.3%, such as at least 99.4%, e.g. at least 99.5%, such as at least 99.6%, e.g. at least 99.7%, such as at least 99.8%, e.g. at least 99. % and preferably 99.7% sequence identity to the sequence of SEQ ID NO 1.

It may be appreciated that modified strain of C. sorokiniana is obtained from a wild type strain of Chlorella sorokiniana. The strain and/or the modified strain of C. sorokiniana may be obtained from a wild type strain of C. sorokiniana by mutagenesis. Mutagenesis may be selected from the group consisting of chemical mutagenesis or physical mutagenesis. The physical mutagenesis may be selected from the group consisting of UV light, gamma rays, X-rays and combinations thereof. Chemical mutagenesis may be a mutagenic chemical selected from the group consisting of ethyl methanesulfonate (EMS), N-methyl-N′-nitro-N-nitrosoguanidine (MNNG) and combinations thereof.

Although not preferred the modified strain of the present invention may also be obtained by site-directed mutagenesis such as CRISPR/Cas9.

Thus, The strain and/or the modified strain C. sorokiniana of the present invention may be obtained from a wild type (or mother strain) by means of e.g. genetic engineering, radiation and/or chemical treatment. It is preferred that the strain and/or the modified strain is a functionally equivalent modified strain, e.g. a strain and/or a modified strain that has substantially the same, or improved, properties (e.g. regarding growth rate and/or protein content) as the strain from which it is derived. Such a strain and/or a modified strain is a part of the present invention. Especially, the term modified strain refers to a strain obtained by subjecting a strain of the invention to any conventionally used mutagenization treatment including treatment with a chemical mutagen such as ethyl methanesulfonate (EMS) or N-methyl-N′-nitro-N-nitroguanidine (MNNG) or irradiation such as x-rays, gamma-rays or UV light. A strain and/or a modified strain may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred modified strain, less than 5%, or less than 1% or even less than 0.1% of the nucleotides in the algae genome have been shifted with another nucleotide, or deleted, compared to the wild type and/or mother strain.

It may be preferred that the strain and/or the modified strain of C. sorokiniana is genetically stable. In the present context “genetically stable” is to be understood as the mutations in the strain and/or the modified strain of C. sorokiniana are stable and do not reverse to the genetics of the wild strain of C. sorokiniana.

In a specific embodiment the wild type strain of C. sorokiniana is UTEX1230.

In a further specific embodiment the strain and/or the modified strain of C. sorokiniana is CCAP 211/136 and mutants and variants thereof. CCAP 211/136 is the strain developed and applied in Examples 1-5 and is in the Examples termed M12.

Another aspect of the present invention relates to a method for producing a strain and/or a modified strain of C. sorokiniana having a chlorophyll content lower than the chlorophyll content of a non-modified wild-type strain of Chlorella sorokiniana when cultured when cultured under the same conditions, wherein said method comprises the steps of:
a) obtaining a parental strain of C. sorokiniana,
b) subjecting the parental strain of | C. sorokiniana to mutagenesis,
c) cultivating the mutated strain of C. sorokiniana on a medium comprising nicotine, norflurazon and/or diphenylamine and
d) identifying colonies of the mutated strain of C. sorokiniana having a phenotype different from the parental strain of C. sorokiniana as the strain and/or the modified strain of C. sorokiniana, and
e) obtaining the strain and/or the modified strain of Chlorella sorokiniana

A further step of:
f) verifying and comparing sequence of chloroplast genome from wild type and the modified C. sorokiniana
   may be included in the method of the present invention.

It will be appreciated that the term “parental strain” is used interchangeably with the term “mother strain”. The “parental strain” may be a wild type strain such as but not limited to UTEX1230.

In an embodiment the culturing in step c) is performed without the presence of natural or artificial light.

In an embodiment the parental strain of C. sorokiniana is a wild-type strain of C. sorokiniana or a variant of a wild-type strain of C. sorokiniana and wherein the mutagenesis is selected from the group consisting of chemical mutagenesis and physical mutagenesis. The physical mutagenesis may be selected from the group consisting of UV light, gamma rays, X-rays and combinations thereof.

The chemical mutagenesis may be performed by exposure of the parental strain of C. sorokiniana to a mutagenic chemical selected from the group consisting of ethyl methanesulfonate, N-methyl-N′-nitro-N-nitroguanidine (MNNG) and combinations thereof. In an embodiment the exposure of the parental strain of C. sorokiniana to the mutagenic chemical in the range of 0-500 mM of the mutagenic chemical, such as in the range from 100-400 mM, e.g. in the range from 200-300 mM and preferably in the range from 200-400 mM of the mutagenic chemical.

In an embodiment step c) is performed at a temperature in a range of 20 to 35° C., and in a for a period of time of 2 to 5 weeks. It may be preferred that the temperature is 28° C., preferably 17 days.

To promote heterotrophic growth the media may comprise an organic carbon energy source selected from the group consisting of glucose, acetate, glycerol and combinations thereof. Likewise it may be contemplated that the media further comprises a nitrogen source selected from the group consisting of urea, nitrate and combinations thereof. The media may be a solid or liquid media. The solid and liquid media may be any media known to the skilled person. The liquid media may for example be media disclosed for high cell density culture defined in Sansawa and Endo 2004 (i.e. a media prepared by dissolving 7 g glucose, 1.0 g KH₂PO₄, 1.0 g MgSO₄·7H₂O, 0.06 g citric acid, 0.009 g FeSO₄·7H₂O, 1.0 ml Arnon's A5, 0.016 g CaCl₂·2H₂O, and 2.5 g urea in 700 ml of tap water). Likewise, the solid media may be the media disclosed for high cell density culture defined in Sansawa and Endo 2004 with the addition of agar. The solid media may for example be prepared such as the liquid media disclosed above (i.e. a media prepared by dissolving 7 g glucose, 1.0 g KH₂PO₄, 1.0 g MgSO₄·7H₂O, 0.06 g citric acid, 0.009 g FeSO₄·7H₂O, 1.0 ml Arnon's A5, 0.016 g CaCl₂·2H₂O, and 2.5 g urea in 700 ml of tap water) and additionally adding 1.5-2% w/w agar. During cultivation, a feeding media may be used. The feeding media may be any media known to the skilled person such as for example the media disclosed for feeding medium-1 defined in Sansawa and Endo 2004 (i.e. a feeding medium prepared by dissolving 171 g glucose, 5.9 g KH₂PO₄, 3.5 g MgSO₄·7H₂O, 1.4 g citric acid, 0.22 g FeSO₄·7H₂O, 3.0 ml Arnon's A5, 0.38 g CaCl₂·2H₂O, and 8.2 g urea in 300 ml of tap water).

The method may comprise an additional step of culturing the strain and/or the modified strain heterotrophically in a liquid media either before or after step (f).

As will be appreciated that phenotype is selected from the group consisting of colour, smell, taste, texture and any combination thereof. In respect of the present invention the phenotype of modified strain of C. sorokiniana may be identified by a colour-shift compared to the colour of the wild-type strain of C. sorokiniana. The colour-shift may be a decrease in the green colour of the strain and/or the modified strain of C. sorokiniana when compared to the wild-type strain of C. sorokiniana when cultured under the same conditions.

Yet an aspect of the present invention relates to a C. sorokiniana obtainable by the above method.

A further aspect of the present invention relates to a method for producing an algae biomass, said method comprising the steps of:
(a) culturing the strain and/or the modified strain of C. sorokiniana of the present invention and/or a composition comprising the strain and/or the modified strain of C. sorokiniana of the present invention aerobic heterotrophically in a media and
(b) obtaining an algae biomass.

In an embodiment the present invention relates to a method for producing an algae biomass, said method comprising the steps of:
(a) culturing the strain and/or the modified strain of C. sorokiniana of the present invention and/or a composition comprising the strain and/or the modified strain of C. sorokiniana of the present invention aerobic heterotrophically in a media and
(b) obtaining an algae biomass, and
(c) storing the algae biomass at 1-32° C. for 1-14 days

In an embodiment storage is performed at a temperature in the range of 1-32° C., such as 2-31° C., e.g. 3-30° C., such as 4-29° C., e.g. 5-29° C., such as 6-28° C., e.g. 7-27° C., such as 8-26° C., e.g. 9-25° C., such as 10-24° C., e.g. 11-23° C., such as 12-22° C., e.g. 13-21° C., such as 14-20° C., e.g. 15-19° C., such as 16-18° C., e.g. 17-19° C. In a further embodiment storage is performed for 1-14 days, such as 2-13 days, e.g. 3-13 days, such as 4-12 days, e.g. 5-11 days, such as 6-10 days, e.g. 7-9 days, such as 8-10 days.

In the present context a heterotroph is an organism that takes in organic carbon as a nutrient source, instead of producing it itself. Aerobic means that oxygen is present. So, when culturing Chlorella aerobic heterotrophically, it states that the cultivation involves the cell to take up organic carbon in the presence of oxygen, which is necessary for the aerobic respiration.

To promote heterotrophic growth the media may comprise an organic carbon energy source selected from the group consisting of glucose, acetate, glycerol and combinations thereof. Likewise it may be contemplated that the media further comprises a nitrogen source selected from the group consisting of urea, nitrate and combinations thereof. The media may be a solid or liquid media. The culturing may be performed without the presence of natural or artificial light.

The culturing in step (a) may be conducted according to Example 1.

In the present context the growth of the C. sorokiniana strain of the present invention was tested by cultivating at photoautotrophic and heterotrophic conditions on Solid Medium, as disclosed in further detail in Example 6.

The method may comprise an additional step d) comprising a step of drying the algae biomass to obtain a dry biomass. The drying step may be performed by any known means in the art such as but not limited to spray drying, vacuum drying, air drying, freeze drying, tray drying and vacuum tray drying.

It may be contemplated that the water activity (Aw) of the dry biomass in the range from 0.01-0.8, preferably in the range from 0.05-0.4.

Aw of a dry biomass is the ratio between the vapor pressure of the dry biomass itself, when in a completely undisturbed balance with the surrounding air media, and the vapor pressure of distilled water under identical conditions. In the present context Aw is measured to 0.4

Yet another aspect of the present invention relates to an algae biomass obtainable by the method of the present invention.

Yet another aspect of the present invention relates to an algae biomass comprising the modified C. sorokiniana of the present invention or a composition comprising the modified C. sorokiniana of the present invention.

An even further aspect of the present invention relates to the use of the modified C. sorokiniana of the present invention or the composition of the present invention for the manufacture of an algae biomass.

It is to be understood that an algae biomass comprising the strain and/or the modified strain of C. sorokiniana and an algae biomass derived from the strain and/or the modified strain of C. sorokiniana are used herein interchangeably.

A still further aspect of the present invention relates to a composition comprising the strain and/or the modified strain of C. sorokiniana of the present invention or an algae biomass comprising the strain and/or the modified strain of C. sorokiniana. In an embodiment the composition may be selected from a concentrate or a dry composition. The composition may comprise a lysate of the strain and/or the modified strain of C. sorokiniana according to the present invention.

In an embodiment the composition may be selected from the non-limiting group consisting of group consisting of foods, nutraceutical preparations or formulations, feed, pharmaceutical compositions including drugs, vaccines, cosmetics, personal care compositions, personal care devices or textiles, dyes, inks, packaging material and combinations thereof.

In an embodiment the composition may be a feed, food, food ingredient and/or a nutritional supplement. In a particular embodiment the composition may be a microalgae ingredient. The microalgae ingredient can be in a powdered form (such as flour), as a liquid or as a paste.

In a further embodiment the composition may be a cosmetic or cosmetic ingredient.

Yet another aspect of the present invention relates to a method for using the composition above as an ingredient in at least one of the group consisting of human foods, human nutraceutical preparations or formulations, animal feeds, pharmaceutical compositions including drugs, vaccines, cosmetics, personal care compositions, personal care devices or textiles, dyes, inks, packaging material and combinations thereof.

A further aspect relates to the use of a strain and/or a modified strain of Chlorella sorokiniana as defined above or an algae biomass comprising the strain and/or the modified strain of Chlorella sorokiniana as defined above in at least one of the group consisting of human foods, food additive, nutraceutical preparations, nutritional supplements or formulations, animal feeds, pharmaceutical compositions including drugs, vaccines, cosmetics, personal care compositions, personal care devices or textiles, dyes, inks and combinations thereof.

A food additive may e.g. be an emulsifier, a gelling agent, foaming agent, protein enrichment or an egg replacement ingredient. Human food may for example be pasta, vegan fish, vegan cold cuts, vegan burger patties, smooties, juices, bread, crispbread, cakes, cookies, purée, meat supplements, vegan dairy supplements and any combination thereof.

A still further aspect relates to the use of a strain and/or a modified strain of Chlorella sorokiniana as defined above, an algae biomass as defined above or a composition comprising the strain and/or the modified strain of Chlorella sorokiniana as defined above or an algae biomass as defined above for increasing the protein content in a food product and/or a feed product.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method for referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the microencapsulated microbial culture and all its features, which may readily be part of the final composition obtained by the method as described herein. Embodiments and features of the present invention are also outlined in the following items and also illustrated by the following non-limiting examples.

### Deposits and Expert Solutions

The applicant requests that a sample of the deposited C. sorokiniana stated in the table below may only be made available to an expert until the date on which the patent is granted.

TABLE 1
Deposits made at a Depositary institution having acquired the
status of international depositary authority under the Budapest
Treaty on the International Recognition of the Deposit of Microorganisms
for the Purpose of Patent Procedure: Culture Collection of Algae
and Protozoa, SAMS Limited, Scottish Marine Institute, OBAN,
Argyll PA37 1QA, Scotland, United Kingdom.

| Strain | Accession No | Deposit date |
|---|---|---|
| Chlorella sorokiniana | CCAP 211/136 | 18 Aug. 2021 |
| M12 | | |

### Sequences

SEQ ID NO 1: chloroplast genome sequence (DNA)-UTEX 1230 wild type C. sorokiniana

### Items

A1a. A strain and/or a modified strain of C. sorokiniana having a chlorophyll content lower than the chlorophyll content of a wild-type strain of C. sorokiniana when cultured under the same conditions

A1b. A strain and/or a modified strain of C. sorokiniana comprising a chlorophyll content at or below 11 mg/g dry cell weight.

A1c. A strain and/or a modified strain of C. sorokiniana having a chlorophyll content lower than the chlorophyll content of a wild-type strain of C. sorokiniana when cultured under the same conditions, and/or wherein the modified strain of C. sorokiniana comprising a content of chlorophyll at or below 11 mg/g dry cell weight

A2. The strain and/or the modified strain of C. sorokiniana according to item Ala wherein the content of chlorophyll is at or below 11 mg/g dry cell weight.

A2a. The strain and/or the modified strain of C. sorokiniana according to items A1b or A2, chlorophyll is chlorophyll a (a-chlorophyll) and/or chlorophyll b (b-chlorophyll).

A3. The strain and/or the modified strain of C. sorokiniana according to any one of the preceding items, wherein the strain and/or the modified strain of C. sorokiniana comprises a chlorophyll A content at or below 7.5 mg/g dry cell weight

A4. The strain and/or the modified strain of C. sorokiniana according to any one of the preceding items, wherein the strain and/or the modified strain of C. sorokiniana comprises a chlorophyll B content at or below 4.5 mg/g dry cell weight

A5. The strain and/or the modified strain of C. sorokiniana according to any one of the preceding items, wherein the strain and/or the modified strain has a protein content of at least 20%, 25%, 30%, 35%, 40%, 45%, 55%, 60% or 65% w/w dry cell weight

A6. The strain and/or the modified strain of C. sorokiniana according to any one of the preceding items, wherein the strain and/or the modified strain comprises a lutein content below 1.5 mg/g dry cell weight

A7. The strain and/or the modified strain of C. sorokiniana according to any one of the preceding items, wherein the strain and/or the modified strain comprises a β-carotene content below 0.33 mg/g dry cell weight

A8. The strain and/or the modified strain of C. sorokiniana according to any one of the preceding items, wherein the strain and/or the modified strain comprises a Neoxanthin content below 0.16 mg/g dry cell weight

A9. The strain and/or the modified strain of C. sorokiniana according to any one of the preceding items, wherein the strain and/or the modified strain of C. sorokiniana comprises at least one mutation in, upstreams or downstreams of one of the genes comprised in the chloroplast genome.

A10. The strain and/or the modified strain of C. sorokiniana according to any one of the preceding items, wherein the chloroplast genome comprises a nucleotide sequence selected from SEQ ID NO: 1

A11. The strain and/or the modified strain of C. sorokiniana according to any one of the preceding claims, wherein the one or more mutations are located in, upstreams or downstreams of a gene associated with:
Photosystem I and photosystem II, and/or
Chloroplast ATP synthase
Chloroplast Clp protease
Transfer RNA

A12. The strain and/or the modified strain of C. sorokiniana according to any one of the preceding items, wherein the one or more mutation(s) are located in the promotor region of any one of the genes comprised in the chloroplast genome

A13. The strain and/or the modified strain of C. sorokiniana according to any one of the preceding items, wherein the one of the genes comprised in the chloroplast genome is selected from the group consisting of:
psbM encoding the protein PSII reaction center protein M
trnS-UAG encoding t-RNA
psbI encoding the protein PSII reaction center protein I
ycf3 encoding PSI reaction center protein Ycf3
atpH encoding ATP synthase subunit c
psaB encoding PSI P700 chlorophyll a apoprotein A2
clpP encoding ATP-dependent Clp protease proteolytic subunit
and/or combinations thereof

A14. The strain and/or the modified strain of C. sorokiniana according to any one of the preceding items, wherein the one or more mutations(s) are located in
(a) atpH and at a position corresponding to position 34544-34554 in SEQ ID NO 1

A15. The strain and/or the modified strain of C. sorokiniana according to any one of the preceding items, wherein the one or more mutations(s) are located upstream of:
(e) psbM and at a position according to 2992 in SEQ ID NO 1,
(f) psbI and at a position according to 29466 in SEQ ID NO 1,
(g) ycf3 and at a position according to 30075-30076 in SEQ ID NO 1, and/or
(h) clp and at a position according to 107103 in SEQ ID NO 1

A16. The strain and/or the modified strain of C. sorokiniana according to any one of the preceding items, wherein the one or more mutations(s) are located downstream of:
c) trnS-UAG and at a position according to 2992 in SEQ ID NO 1, and/or
d) psaB and at a position according to 72653 in SEQ ID NO 1

A17. The strain and/or the modified strain of C. sorokiniana according to any one of the preceding items, wherein the one or more mutations(s) are:
(a) a substitution from G to A at a position corresponding to position 2992 in SEQ ID NO 1,
(b) a substitution from T to A at a position corresponding to position 29466 in SEQ ID NO 1,
(c) a deletion of AA at a position corresponding to position 30075-30076 in SEQ ID NO 1,
(d) a deletion of CAGAAGCAGAA at a position corresponding to position 34544-34554,
(e) a deletion of A at a position corresponding to position 72653 in SEQ ID NO 1 and/or
(f) a deletion of A at a position corresponding to position 107103 in SEQ ID NO 1

A18. The strain and/or the modified strain of C. sorokiniana according to any one of the preceding items, wherein at least one mutation(s) is/are point mutations

A19. The strain and/or the modified strain of C. sorokiniana according to any one of the preceding items, wherein the strain and/or the modified strain of C. sorokiniana is a heterotroph

A20. The strain and/or the modified strain of C. sorokiniana according to any one of the preceding items, wherein the strain and/or the modified strain of C. sorokiniana is obtained from a wild type strain of Chlorella sorokiniana

A21. The strain and/or the modified strain of C. sorokiniana according to any one of the preceding items, wherein the strain and/or the modified strain of C. sorokiniana is obtained from a wild type strain of C. sorokiniana by mutagenesis

A22. The strain and/or the modified strain of C. sorokiniana according to any one of the preceding items, wherein the mutagenesis is selected from the group consisting of chemical mutagenesis, physical mutagenesis (e.g. UV light, x-rays, gamma-rays), site-directed mutagenesis (e.g. CRISPR/Cas9) and combinations thereof

A23. The strain and/or the modified strain of C. sorokiniana according to any one of the preceding items, wherein the chemical mutagenesis is a mutagenic chemical selected from the group consisting of ethyl methanesulfonate (EMS), N-methyl-N′-nitro-N-nitrosoguanidine (MNNG) and combinations thereof

A24. The strain and/or the modified strain of C. sorokiniana according to any one of the preceding items, wherein the strain and/or the modified strain of C. sorokiniana is genetically stable

A25. The strain and/or the modified strain of C. sorokiniana according to any one of the preceding items, wherein the wild type C. sorokiniana is UTEX1230.

A26. The strain and/or the modified strain of C. sorokiniana according to any one of the preceding items, wherein the strain and/or the modified strain of C. sorokiniana is CCAP 211/136 and mutants and variants thereof.

A27. The strain and/or the modified strain of C. sorokiniana according to any one of the preceding items, wherein the strain and/or the modified strain of C. sorokiniana has the mutations of the C. sorokiniana M12 strain (deposit number CCAP 211/136).

A28. The strain and/or the modified strain of C. sorokiniana according to any one of the preceding items, wherein the strain and/or the modified strain of C. sorokiniana is the C. sorokiniana strain according to deposit number CCAP 211/136.

B1. A method for producing a strain and/or a modified strain of C. sorokiniana having a chlorophyll content lower than the chlorophyll content of a non-modified wild-type strain of Chlorella sorokiniana when cultured under the same conditions, wherein said method comprises the steps of:
a) obtaining a parental strain of C. sorokiniana,
b) subjecting the parental strain of C. sorokiniana to mutagenesis,
c) cultivating the mutated strain of C. sorokiniana on a medium comprising nicotine, norflurazon and/or diphenylamine and
d) identifying colonies of the mutated strain of C. sorokiniana having a phenotype different from the parental strain of C. sorokiniana as the strain and/or the modified strain of C. sorokiniana, and
e) obtaining the strain and/or the modified strain of Chlorella sorokiniana

B2. The method according to item B1, wherein the parental strain of C. sorokiniana is a wild-type strain of C. sorokiniana or a variation of a wild-type strain of C. sorokiniana and wherein the mutagenesis is selected from the group consisting of chemical mutagenesis, physical mutagenesis, site-directed mutagenesis and combinations thereof

B3. The method according to any one of items B1-B2, wherein the chemical mutagenesis is performed by exposure of the parental strain C. sorokiniana to a mutagenic chemical selected from the group consisting of ethyl methanesulfonate, N-methyl-N′-nitro-N-nitrosoguanidine and combinations thereof

B4. The method according to any one of items B1-B3, wherein said exposure of the mutagenic chemical in the range of 0-500 mM of the mutagenic chemical.

B5. The method according to any one of items B1-B4, wherein the physical mutagenesis is selected from the group consisting of UV light, gamma rays, X-rays and combinations thereof

B6. The method according to any one of items B1-B5, wherein step b) is performed at room temperature and for a period of time between 30 min-3 hours, preferably 1 hour

B7. The method according to any one of items B1-B6, wherein step c) is performed for 2 to 5 weeks, preferably 17 days

B8. The method according to any one of items B1-B7, wherein said media further comprises an organic carbon energy source selected from the group consisting of glucose, acetate, glycerol and combinations thereof

B9. The method according to any one of items B1-B8, wherein said media further comprises a nitrogen source selected from the group consisting of urea, nitrate and combinations thereof

B10. The method according to any one of items B1-B9, wherein the phenotype is selected from the group consisting of colour, smell, taste, texture and combinations thereof.

B11. The method according to any one of items B1-B10, wherein the phenotype of mutated strain of C. sorokiniana is identified by a colour-shift compared to the colour of the wild-type strain of Chlorella sorokiniana

C1. A C. sorokiniana obtainable by the method according to any one of items B1-B11

D1. A method for producing an algae biomass, said method comprising the steps of:
(a) culturing the strain and/or the modified strain of C. sorokiniana according to any one of items Ala-A28 or Alb-A28 and/or a composition according to any one of items H1-H7 aerobic heterotrophically in a media and
(b) obtaining an algae biomass

D1a. A method for producing an algae biomass, said method comprising the steps of:
(a) culturing the strain and/or the modified strain of C. sorokiniana according to any one of items A1a-A28 or A1b-A28 and/or a composition according to any one of items H1-H7 aerobic heterotrophically in a media and
(b) obtaining an algae biomass
(c) storing the strain and/or the modified strain at 1-32° C. for 1-14 days

D2. The method according to item D1, wherein the media comprises an organic carbon energy source selected from the group consisting of glucose, acetate, glycerol and combinations thereof

D3. The method according to any one of items D1-D2, wherein the media comprises a nitrogen source selected from the group consisting of urea, nitrate and combinations thereof

D4. The method according to any one of items D1-D3, wherein the method further comprises a step of drying the algae biomass

E1. An algae biomass obtainable by the method according to any one of items D1-D4

F1. An algae biomass comprising the modified C. sorokiniana according to any one of items A1-A28 or the composition the composition according to any one of items H1-H7

G1. Use of the modified C. sorokiniana according to any one of items A1a-A28 or A1b-A28 or the composition according to any one of items H1-H7 for the manufacture of an algae biomass.

H1. A composition comprising a strain and/or a modified strain of C. sorokiniana according to any one of items A1a-A28 or A1b-A28 or an algae biomass comprising the strain and/or the modified strain of C. sorokiniana according to item F1.

H2. The composition according to item H1, wherein the composition is selected from a concentrate or a dry composition.

H3. The composition according to any one of items H1-H2, wherein the composition comprises a lysate of the strain and/or the modified strain of C. sorokiniana according to any one of items A1a-A28 or A1b-A28.

H4. The composition according to any one of items H1-H3, wherein the composition is a feed, food, food ingredient and/or a nutritional supplement.

H5. The composition according to any one of items H1-H4, wherein the composition is a ingredient.

H6. The composition according to any one of items H1-H5, wherein the composition is a cosmetic or cosmetic ingredient.

H7. The composition according to any one of items H1-H6, wherein the composition is pharmaceutical compositions selected from the group consisting of drugs, vaccines, cosmetics, personal care compositions, personal care devices or textiles, dyes, inks, packaging material and any combination thereof.

I1. A method for using the composition according to any one of items H1-H7 as an ingredient in at least one of the group consisting of human foods, human nutraceutical preparations or formulations, animal feeds, pharmaceutical compositions including drugs, vaccines, cosmetics, personal care compositions, personal care devices or textiles, dyes, inks, packaging material and combinations thereof.

J1. Use of a strain and/or a modified strain of C. sorokiniana according to any one of items A1a-A28 or A1b-A28 or an algae biomass comprising the strain and/or the modified strain of C. sorokiniana according to item F1 in at least one of the group consisting of human foods, nutraceutical preparations, nutritional supplements or formulations, animal feeds, pharmaceutical compositions including drugs, vaccines, cosmetics, personal care compositions, personal care devices or textiles, dyes, inks, packaging material and combinations thereof.

k1. Use of a strain and/or a modified strain of C. sorokiniana according to any one of items A1a-A28 or A1b-A28 or an algae biomass derived from the strain and/or the modified strain of C. sorokiniana according to item F1 for increasing the protein content in a food product and/or a feed product

### REFERENCES

Wright S, Jeffrey S, Mantoura R, Llewellyn C, Bjornland T, Repeta D, Welschmeyer N, 1991. Improved HPLC method for the analysis of chlorophylls and carotenoids in marine phytoplankton. Marine Ecology Progress Series 77:183-196.
Jeffrey S. W., Wright S. W. Zapata M. (1999) Recent advances in HPLC pigment analysis of phytoplankton. Marine and Freshwater Research 50, 879-896. https://doi.org/10.1071/MF99109
Goericke R Repeta D J (1993) Chlorophylls a and b and divinyl chlorophylls a and b In the open subtropical North Atlant˜c Ocean Mar Ecol Prog Ser 101 307-313
idussi F. Claustre H, Bustillos-Guzmbn J, Cailliau C, Marty J C (1996) Determination of chlorophylls and carotenoids of marine phytoplankton: separation of chlorophyll a from divinyl chlorophyll a and zeaxanthin from lutein. J Plankton Res 18:2377-2382
Rarlow R G, Mantoura R F C, Cummings D G, F˜lernan TW (1997a) Plgment chemotaxonom˜c d˜str˜butions of phytoplankton dunng summer In the western Med˜terranean Deep Sea Res 11 44 833-850
Zapata M, Rodriguez F, Garrido J L 2000. Separation of chlorophylls and carotenoids from marine phytoplankton: a new HPLC method using a reversed phase C8 column and pyridine-containing mobile phases. Marine Ecology Progress Series 195:29-45.
Bidigare, R. R., Van Heukelem, L., Trees, C. C., 2005. Analysis of algal pigments by high-performance liquid chromatography. In: Andersen, R. (Ed.), Algal Culturing Techniques. Academic Press, pp. 327-345.
Bidigare, R. R., Trees, C. C., 2000. HPLC phytoplankton pigments: sampling, laboratorymethods, and quality assurance procedures. In: Mueller, J. L., Gargion, G. (Eds.), Ocean Optics Protocols for Satellite Ocean Color Sensor Validation, Revision 2, NASA Technical Memo, 2000209966, pp. 154-161
Van Heukelem L., Thomas C. S. 2001. Computer-assisted high-performance liquid chromatography method development with applications to the isolation and analysis of phytoplankton pigments. Journal of Chromatography A 910:31-49
Chen and Meyers. 1984. A rapid quantitative method for determination of astaxanthin pigment concentration in oil extracts. JAOCS, 61, 1045-1047
Sansawa and Endo, 2004, Production of Intracellular Phytochemicals in Chlorella under Heterotrophic Conditions, Journal of Bioscience and Bioengineering, Vol. 98, No. 6, 437-444.

### EXAMPLES

### Example 1: Mutagenic Exposure of C. sorokiniana to Obtain a Modified Strain of C. sorokiniana with Lowered Chlorophyll Content

### Purpose

In order to develop a modified strain of Chlorella sorokiniana with a lowered chlorophyll content, a wild-type strain (Chlorella sorokiniana UTEX 1230) was exposed to a chemical mutagen to induce random mutations.

### Method:

Mutagenesis of Chlorella sorokiniana: To induce random mutations, the mutagenic chemical ethyl methanesulfonate (EMS) was used. Cultures were inoculated in liquid medium (comprising glucose as a carbon-source, urea as a nitrogen source, KH₂PO₄, MgSO₄·7H₂O, citric acid, FeSO₄·7H₂O, Arnon's A5 and CaCl₂·2H₂O (Sansawa and Endo, 2004), hereafter referred to as “Liquid Medium”) with growing biomass from solid medium (comprising the same components as the liquid medium and agar, hereafter referred to as “Solid Medium”). The cultures were incubated for 25-35 hours in a shaking incubator at 28-35° C. and 150 rpm. The cultures were checked for contamination at 400× magnification and pooled. The cell concentration of the pooled cultures was determined using a cell counter (CytoSMART Technologies). The cell concentration of the culture was concentrated to 1·10⁹ cells/ml in Liquid Medium. These cultures were exposed to 0-400 mM sterile EMS for 30-180 min in the dark under constant gentle inversion on a HulaMixer (Invitrogen, ThermoFischer Scientific) at RT. The mutagenesis was stopped by washing the cells three times in Liquid Medium. At the final washing, cells were resuspended in Liquid Medium.

Spray-plating: To select and isolate mutants, growth in colonies originating from single cells was necessary. To obtain growth in single-cell colonies, the algal cells were distributed evenly on Solid Medium comprising nicotine using spray-plating. The algae culture used for spray-plating was 1·10⁸-1·10⁹ cells/ml. A glass capillary with a diameter of 1-3 mm reached the algae-culture in an Eppendorf tube and the culture is pulled up in the capillary, forming a droplet on top of the capillary. A sterile airstream (˜4 l/min) was positioned towards the droplet on top of the capillary, and separated the droplet into smaller droplets, separating the cells, which landed on the Solid Medium comprising nicotine. The plates were incubated at 25-30° C. in the dark for 10-20 days.

### Result

Single cell colonies were visible on all plates except the plates containing cells exposed to 400 mM EMS. A number of colonies were isolated from cultures exposed to 200 mM and 300 mM EMS, as these colonies had yielded a phenotype different from the wild type.

### Example 2: Identification of a Modified Strain of C. sorokiniana

### Purpose

To identify a modified strain of C. sorokiniana from the wild type after mutagenesis, based on phenotype.

### Method

Selection of colonies: Colonies expressing a color-phenotype different from the wild type were isolated by visual observation and transferred into Liquid Medium, which were incubated for 3-5 days in a shaking incubator at 28-35° C. and 150 rpm. After 1-3 days, fresh Liquid Medium was added to each culture. Cultures with a phenotype different from the wild type were plated on Solid Medium. The biomass was incubated and stored at 25-32° C. in the dark. Colonies with a color-phenotype different from the wild type were selected and the biomass was transferred into Liquid Medium in Erlenmeyer culture flasks with filtered lids and incubated at 28-35° C. and 150 rpm for 2-5 days.

### Result

After 2-5 days of incubation, mutated colonies yielded biomass with a color phenotype different from the wild type, indicating an altered pigment content. A modified strain of Chlorella sorokiniana was isolated (in the following termed M12). M12 showed a phenotypic pale green color, different from the phenotypic dark green color in the wild type strain of Chlorella sorokiniana. M12 was stable during several cultivations.

### Example 3: Monitor Growth and Obtaining an Algae Biomass from the Modified Strain of C. sorokiniana (M12)

### Purpose

In order to monitor the growth of M12 and the wild type strain of Chlorella sorokiniana, these were cultivated in a Liquid Medium under heterotrophic conditions. The dry weight, glucose and cell concentration was measured during the heterotrophic growth.

### Method

Heterotrophic cultivation: Cultures with growing biomass from the wild type and M12 from Solid Medium were inoculated in 250 ml baffled Erlenmeyer flasks with filtered lids containing Liquid Medium and incubated at 28-35° C. and 150 rpm in a shaking incubator for 2-5 days in the dark. The cultures were transferred to 1000 ml baffled Erlenmeyer flasks with filtered lids containing fresh Liquid Medium and incubated at 28-35° C. and 150 rpm in a shaking incubator for 2-5 days in the dark. Growth of the wild type and M12 were further monitored during cultivation in 3-liter bioreactors.

The cultures in 1000 ml baffled Erlenmeyer flasks with filtered lids containing the wild type and M12 were each added to a 3-liter bioreactor (Shanghai Bailun biological technology co., Ltd.) containing Liquid Medium. The medium was calibrated to a pH of 6-7.5, which was monitored with a pH-sensor and automatically adjusted with NaOH and the dissolved oxygen level was adjusted to 70-75%, which was monitored with a DO-sensor and automatically adjusted with an automatic airflow rate at ˜1 vvm and coupled stirring. A foam sensor in the bioreactors controlled the foaming level automatically using an antifoaming agent (2% Struktol SB 2239 A). During the heterotrophic cultivation in the bioreactors, feeding medium (comprising glucose as a carbon-source, urea as a nitrogen source, KH₂PO₄, MgSO₄·7H₂O, citric acid, FeSO₄·7H₂O, Arnon's A5 and CaCl₂·2H₂O (Sansawa and Endo, 2004), hereafter referred to as “Feeding Medium” was added as the glucose concentration of the media reached ˜0 g/l. Feeding Medium was added during the growth of the wild type and M12. The bioreactors were kept at a temperature of 28-35° C. and the bioreactor containing the mutant was darkened using aluminum foil.

Measurement of dry weight, glucose and cell concentration: Growth of the wild type and M12 was monitored during heterotrophic cultivation. The dry weight of the cultures was measured using a moisture analyzer (PCE Instruments) and the cell concentration was measured using a cell counter (CytoSMART Technologies). The glucose level of the media was monitored during the heterotrophic growth using the D-Glucose Assay Kit-GOPOD format (Megazyme Ltd.) following the manufacture's protocol.

The biomass yield from glucose was calculated using the equation:

$$Y = \frac{Biomass end - Biomass start}{Glucose start - Glucose end}$$

Y is the yield, and the biomass is either measured on the total dry weight of the biomass (g) or the total number of cells. The start-glucose is the total amount of available glucose calculated from the known glucose concentration of liquid medium and volumes, and the end-glucose is the total amount of remaining glucose at the end of cultivation calculated from the measured end-glucose concentration and volumes.

### Result

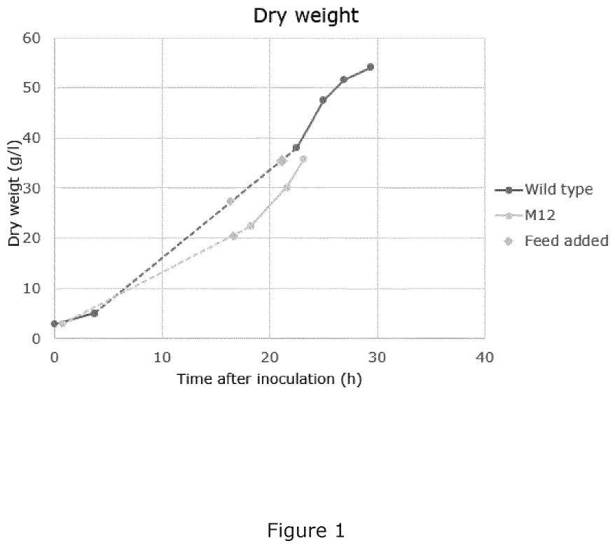

During cultivation in baffled Erlenmeyer flasks, the cell concentration and the dry weight was increasing in both M12 and wild type Chlorella sorokiniana, as can be seen in the table 2 below and in FIG. 1. The dry weight yield from glucose of the wild type was 0.47 g·g⁻¹ and 0.42 g·g⁻¹ of M12 and the cell yield from glucose of the wild type was 6.0·10¹⁰ cells·g⁻¹ and 5.48·10¹⁰ cells·g⁻¹ of M12. Thus, the yield from glucose of the wild type and M12 resembled each other.

TABLE 2
Growth of wild type and M12 during cultivation
in baffled Erlenmeyer flasks

| | Wild type strain | Modified strain (M12) |
|---|---|---|
| Cells - start (number of cells) | 5.35 · 10⁸ | 3.94 · 10⁸ |
| Cells - end (number of cells) | 2.71 · 10¹¹ | 2.47 · 10¹¹ |
| Dry weight - start (g) | 0.14 | 0.075 |
| Dry weight - end (g) | 2.25 | 1.97 |
| Glucose available (g) | 4.5 | 4.5 |
| Glucose remaining (g) | 0 | 0.008 |
| Y_{Dry weight} (g_{biomass} / g_{glucose}) | 0.47 | 0.42 |
| Y_{Cell} (cells / g_{glucose}) | 6.0 · 10¹⁰ | 5.48 · 10¹⁰ |

During cultivation in 3-liter bioreactors, the M12 strain has a higher dry weight and cell yield than the wild type, as can be seen in table 3 below.

The dry weight yield from glucose of the wild type was 0.46 g·g⁻¹ and 0.72 g·g⁻¹ of the M12, and the cell yield from glucose of the wild type was 2.31·10¹⁰ cells·g⁻¹ and 3.22·10¹⁰ cells·g⁻¹ of the strain and/or the M12 in table 3. The M12 retained its pale green colour throughout the cultivation period.

TABLE 3
Growth of wild type and M12 during
cultivation in 3-liter bioreactors.

| | | Modified strain |
|---|---|---|
| | Wild type strain | (M12) |
| Y_{Dry weight (g biomass / g glucose)} | 0.46 | 0.72 |
| Y_{Cell (cells / g glucose)} | 2.31 · 1010 | 3.22 · 1010 |

### Example 4: Verify and Comparing Sequence of Chloroplast Genome from Wild Type and the Modified C. sorokiniana

### Purpose

To verify that M12 could still be classified as Chlorella sorokiniana UTEX 1230, the genome of the wild type strain and M12 were sequenced using Nanopore sequencing. The quality and quantity of high molecular weight (HMW) DNA from the wild type strain and the M12 was measured.

### Method

Cell preparation for sequencing: Prior to sequencing, high molecular weight (HMW) DNA was extracted and purified from algal biomass. Cultures with the wild type and M12 were inoculated in liquid medium in Erlenmeyer culture flasks with filtered lids and incubated at 28-35° C. and 150 rpm overnight.

DNA extraction: The cultures were examined for contamination and DNA was extracted from the algal biomass of M12 and the wild type. The DNA was purified using QIAGEN Genomic Tips 20/G, which were used following the QIAGEN Genomic DNA Preparation protocol. Small fragments of DNA were removed from the extracted DNA, leaving only High Molecular Weight (HMW) DNA, by using the Circulomics Short Read Eliminator XS kit following the manufacturer's protocol.

The quantity of the HMW DNA was measured on a spectrophotometer (NanoDrop™ One/OneC Microvolume UV-Vis Spectrophotometer, ThermoFischer Scientific) and a Qubit 4 Fluorometer (ThermoFischer Scientific) using the Qubit™ dsDNA HS Assay Kit following the manufacturer's protocol. The quality of the HMW DNA was determined with an electrophoretic analysis on a 2200 TapeStation (Agilent Technologies, Inc.) using the Agilent Genomic DNA ScreenTape (Agilent Technologies, Inc.) following the manufacturer's protocol, by determining the length of the DNA fragments and the DNA Integrity Number (DIN).

Nanopore sequencing of high molecular weight DNA: The HMW DNA was sequenced using Nanopore sequencing. Preparation of the HMW DNA was carried out using the Ligation Sequencing Kit-SQT-LSK109 (Oxford Nanopore Technologies) following the manufacturer's protocol, and the Long Fragment Buffer was used to enrich DNA fragments >3 kb. The prepared HMW DNA was sequenced using an R10.3 (FLO-MIN106D) MinION Flow Cell (Oxford Nanopore Technologies) that were primed and loaded using the Flow Cell Priming Kit-EXP-FLP002 (Oxford Nanopore Technologies) following the manufacturer's protocol.

Raw reads from the Nanopore sequencing were processed using Guppy v4.2.2 (Oxford Nanopore Technologies, Oxford, UK) in GPU mode using the dna_r9.4.1_450bps_hac.cfg model, Filtlong v0.2.0 (https://github.com/rrwick/Filtlong) to a minimum length of 10 kbp and a minimum quality of 80, Minimap2 v2.17 (Li, 2018), Minimap2 v2.17 (Li, 2018), Miniasm v0.3 (Li, 2016), Racon v1.3.3 (Vaser et al., 2017) and Medaka v1.0.1 (https://github.com/nanoporetech/medaka).

QIAGEN CLC Genomics Workbench 20.0 (https://digitalinsights.qiagen.com/) was used to align chloroplast genomes and identify differences between sequences. A reference sequence (Accession number: KJ742376.1) was used to identify the location of genes in the chloroplast genomes of the wild type and mutants.

### Result

M12 and the wild type strain had above 99.9% sequence similarity in the chloroplast genomes and thereby taxonomically classifying M12 as Chlorella sorokiniana UTEX 1230.

Six mutations were detected in the chloroplast genome of the modified strain of Chlorella sorokiniana (M12) compared to the chloroplast genome of the wild type strain of Chlorella sorokiniana.

Two of the mutations were a deletion of one nucleotide, two of the mutations were a substitution of one nucleotide to another, one mutation was a deletion of two consecutive nucleotides and one mutation was a deletion of 11 consecutive nucleotides.

Some of the genes affected by the mutations in M12 are transcribed to proteins associated with photosystem I and II (PSI and PSII), ribosomes in the chloroplasts, chloroplastic ATP synthase, enzymes necessary for the production of chlorophyll and chloroplasts resulting in the pale green phenotype by knocking out pigmentation genes in the algae.

### Example 5: Pigment and Protein Analysis of the Modified Strain of C. sorokiniana (M12)

Summarizing the above. In order to develop a modified strain of Chlorella sorokiniana with a lowered chlorophyll content, a wild type strain (Chlorella sorokiniana UTEX 1230) was exposed to a chemical mutagen to induce random mutations.

The wild type of Chlorella sorokiniana was exposed to the mutagenic chemical ethyl methanesulfonate (EMS) and cultivated on a medium containing nicotine and incubated.

The modified strain of Chlorella sorokiniana (M12) was selected visually by comparing phenotypic color of the modified strain and wild type. The modified strain of Chlorella sorokiniana was isolated (M12) and showed a phenotypic bright color, different from the phenotypic color of dark green in the wild type strain of Chlorella sorokiniana. The color of the modified strain of Chlorella sorokiniana (M12) was stable during several cultivations.

In order to monitor the growth of the modified strain (M12) and the wild type strain of Chlorella sorokiniana, the modified strain (M12) and the wild type strain of Chlorella sorokiniana was cultivated in a media composition using glucose as carbon-source and urea as nitrogen-source (see FIG. 1).

The dry weight of the cultures was measured of the modified strain (M12) and the wild type strain of Chlorella sorokiniana during the heterotrophic growth. The increase of dry weight in the modified strain of Chlorella sorokiniana (M12) was similar to the wild type strain of Chlorella sorokiniana.

### Purpose

The protein content of the modified strain of Chlorella sorokiniana (M12) was measured after storage at 3-6° C. for 5-10 days to compare it to the protein content of the wild type strain of Chlorella sorokiniana. The pigment content of the modified strain of Chlorella sorokiniana (M12) was also measured on fresh biomass and on biomass after storage at 3-6° C. for 5-10 days to compare it to the pigment content of the wild type strain of Chlorella sorokiniana.

The protein content was measured using the Kjeldahl method.

The protein content of the modified strain of Chlorella sorokiniana (M12) was 36.9% compared to 55% of the wild type strain of Chlorella sorokiniana. Although the protein content of M12 was lower than the wild type, M12 still showed an elevated protein level compared to other vegan protein-sources such as chickpeas (˜19%), lentils (˜26%), edamame beans (˜12%) and kidney beans (˜24%).

As can be seen in the table below no pigments were present in a detectable amount in stored biomass of the modified strain of Chlorella sorokiniana (M12) compared to several pigments present in the wild type strain of Chlorella sorokiniana. In the fresh biomass of the modified strain of Chlorella sorokiniana (M12), no pigments were present in a detectable amount, except for lutein, which was barely detectable with a level of <50 ng/ml.

TABLE 4
pigment content

| | | Modified strain | Modified strain |
|---|---|---|---|
| | | (M12) Fresh | (M12) Stored |
| | Wild type strain | biomass | biomass |
| Chlorophyll A | 7.3 mg/g | Below detection | Below detection |
| Chlorophyll B | 4.3 mg/g | Below detection | Below detection |
| Lutein | 1.5 mg/g | <50 ng/ml | Below detection |
| β-carotene | 0.33 mg/g | Below detection | Below detection |
| Neoxanthin | 0.16 mg/g | Below detection | Below detection |
| Unidentified | 0.76 mg/g | Below detection | Below detection |
| carotenoids | | | |

To compare the chloroplast genomes of the wild type strain and modified strain of Chlorella sorokiniana (M12), the chloroplast genomes were sequenced using Nanopore Sequencing.

Six mutations were detected in the chloroplast genome of the modified strain of Chlorella sorokiniana compared to the chloroplast genome of the wild type strain of Chlorella sorokiniana. Two was a deletion of one nucleotide, one was a deletion of two consecutive nucleotides and one was a deletion of 11 consecutive nucleotides. Two was a substitution of one nucleotide. Genes related to Photosystem I and II, chloroplast ATP synthase, Chloroplast Clp protease and Transfer RNA Genetic were affected by the mutations. Genetic analyses revealed that the similarity between the chloroplast genomes of the modified strain and the wild type strain of Chlorella sorokiniana was above 99.9%, and thereby taxonomically classifying the modified strain of Chlorella sorokiniana as Chlorella sorokiniana UTEX 1230.

### Method Used for Pigment Analysis

Carotenoids and chlorophylls were measured as follows:

Between 10-100 mg of dried microalgal powder was weighed off and mixed with 850 mg glass beads in a tube. A mixture of acetone and methanol was added. The mixture was vortexed for less than 1 minute and centrifuged 10-20 min at 1500 rpm. Subsequently the solvent layer was transferred to a new tube and the extraction was repeated two more times on the remaining pellet. All solvent fractions were combined and filtered through at 0.22 μm filter.

The extracts were analyzed using HPLC with 70% Methanol+30% 0.028 M Tetrabutyl ammonium acetate (TBAA) in water and pure methanol was mobile phases. Pigment extracts were diluted in a buffer containing TBAACC solution in water and methanol. Separation took place over 45 min with a gradient of methanol from 5% to 95% over 27 min, 1 min to 100%, 3 min at 100%, 2 min to 5%, 6 min at 5%. Quantification of the pigments was performed by measuring using standard certified pigments of known concentration and comparing the samples to this.

### 2. Definition

This method measures carotenoids and chlorophylls, which are extractable by acetone and methanol (7+3).

Both carotenoids and chlorophylls are light harvesting pigments important for photosynthesis of plants and algae. They contribute to the colour of plants and algae. Carotenoids belong to the category of tetraterpenoids (i.e., they contain 40 carbon atoms, being built from four terpene units each containing 10 carbon atoms). Structurally, carotenoids take the form of a polyene hydrocarbon chain, which is sometimes terminated by rings, and may or may not have additional oxygen atoms attached.

Carotenoids with molecules containing oxygen, such as lutein and zeaxanthin, are known as xanthophylls.

The unoxygenated (oxygen free) carotenoids such as α-carotene, β-carotene, and lycopene, are known as carotenes. Carotenes typically contain only carbon and hydrogen (i.e., are hydrocarbons), and are in the subclass of unsaturated hydrocarbons.

Chlorophylls are numerous in types, but all are defined by the presence of a fifth ring beyond the four pyrrole-like rings. Most chlorophylls are classified as chlorins, which are reduced relatives to porphyrins (found in hemoglobin). They share a common biosynthetic pathway as porphyrins, including the precursor uroporphyrinogen III. Unlike hemes, which feature iron at the centre of the tetrapyrrole ring, chlorophylls bind magnesium. The chlorin ring can have various side chains, usually including a long phytol chain. The most widely distributed form in terrestrial plants is chlorophyll a. The concentration of each of the pigments analysed is expressed in μg/g sample.

### 3. Field of Application

All types of dried microalgae, seaweed and their products (see note 11.2).

### 4. Principle

The carotenoids and chlorophylls from different microalgae and seaweed species are extracted by using acetone and methanol as a solvent and glass beads plus vortexing to disintegrate the cell membranes and liberate the pigment content. By repeating the extraction process four times, the extracts are being collected and analysed by high-performance liquid chromatography (HPLC) analysis using photodiode array detector.

The pigment concentration of the extract is determined based on the pigment identities (retention times) and the peak areas.

### 5. Equipment

5.1 Analytical balance (4 decimals)
5.2 Finn pipettes (1-5 ml) and tips.
5.3 15 and 50 ml plastic test tubes with lid
5.4 HPLC column: Eclipse XDB-C8 stainless steel 4.6×150 mm, with 3.5 μm particle size and proper guard: Eclipse XDB-C8 4.6×12.5 mm, 3.5 μm (Agilent Technologies)
5.5 PTFE syringe filter 0.22 μm (e.g. no. 15161499, Fisher Scientific Biotech Line).
5.6 Spectrophotometer (wavelength range from 400-800 nm) 5.7 Quartz cell 1 cm width.

5.8 HPLC unit including pump, column oven, PDA (photo diode array detector) and programmable auto sampler equipped with cooling unit (4° C.).

5.9 Centrifuge (Sigma 4K15, rotor 11150)
5.10 Vortex mixer
5.11 Glass beads (Ø0.75 mm-1 mm)

### 6. Reagents

6.1 Methanol (HPLC grade) Cas. no. 67-56-1
6.2 Acetone (HPLC grade) Cas. no. 67-64-1
6.3 Deionized water (Millipore treated water)
6.4 Tetrabutyl ammonium acetate, 0.028 M in water (TBAA, Cas. no. 10534-59-5). Weigh 2.53 g TBAA and add water up to 300 ml (6.3)
6.5 Tetrabutyl ammonium acetate, 0.028 M in water pH 6.5. Weigh 0.75 g TBAA and add 90 ml water. Adjust pH by HCl or NaOH and add water up to 100 ml.
6.6 Pigment standards for calibration curve:
6.6.1 Pigments in known concentrations (in 90% acetone or ethanol) from DHI: Concentration range ca. 1.0 to 3.0 μg/ml. See table 5
6.6.2 Pigments in powder form from other companies: For preparation of parent solution of these compounds, see 8.9.
   Astaxantin Dr. Ehrenstorfer, CA10307000
   Canthaxanthin, Fluka 32993
   Lycopene, Sigma-Aldrich 75051
6.7 Pigment mix (From DHI) MIX-1 ca 3 μg/ml (see Table 4)
6.8 Acetone+Methanol (7+3)
6.9 Mobile phase A: 70% methanol+30% of 0.028 M TBAA in water: 700 ml (6.1) and 300 ml (6.4) is mixed.
6.10 Buffer. 90% of 0.028 M TBAA in water, pH 6.5+10% methanol: 90 ml TBAA (6.5) and 10 ml methanol (6.1) is mixed. Can be poured into vials and stored in the −18° C. freezer until use

### 7. Handling of Sample

Sample shall be received freeze dried and analysed immediately or stored at −20° C. and dark prior to the analysis (See note 11.2). The analysis should be performed within one month, since storage can decay pigments, even though stored frozen.

It is highly recommended to do all the analysis process in a place out of light.

### 8. Procedure

Extraction is made at least in duplicate and the HPLC analysis is made as single determination on each extract. For statistical purposes, triplicates are recommended.

### Extraction:

8.1 Weigh approx. 25.0 mg of dried microalgae or approx. 100.0 mg of dried seaweed and other products containing microalgae in a 15 ml plastic tube with lid (5.3), add approx. 850 mg glass beads (1 small spoon).
8.2 Add 5 ml of acetone+methanol (6.8).
8.3 Vortex during 30 seconds
8.4 Centrifuge sample at 1500 rpm (447 g) for 10 min and transfer solvent layer to a 50 mL test tube using a pipette
8.5 Repeat step 8.2 to step 8.4 three times to obtain completely clear sediment. For microalgae it may not be possible to obtain a completely white sediment. Combine the supernatants containing the pigments in the 50 ml test tube. Final volume will be around 20 ml.
8.6 Filter sample prior to the analysis by syringe filter 0.22 μm (5.5).
8.7 Extracted sample shall be analyzed immediately and in case that is not possible be kept at −18° C. and away from light. Shelf life at −18° C. has not been investigated.

### Determination of Concentration of Standards to be Used for the Calibration Curve:

8.8 Pigment standards (6.6.1) can be purchased in solution with known concentrations provided from the DHI Water & Environment (parent solution). The concentrations of these standards do not need to be checked when a new batch of pigment is received. If the pigment has been kept for 1 year or longer, then the concentration should be checked as described under 8.10. If the concentration of a given pigment differs by more than 10% from the concentration stated by DHI, then a new calibration curve should be made using the newly determined concentration (see Preparation of calibration curves (8.11 and 8.12)).
8.9 Solution of pigment standards (6.6.2) can also be prepared from single standard pigments in powder form, e.g. astaxanthin and canthaxanthin. Make a parent solution in acetone+methanol (7+3) (6.8) with a concentration of the standard of 1 μg/ml, far away from direct light in fume hood. Concentrations of these solutions must be determined spectrophotometrically as described in 8.10.
8.10 Absorbance (Amax) is measured in a 1-cm cuvette at the standard's wavelength λmax (See table 4) and at 750 nm to correct for light scattering. Note that the absorbance at known λmax of the pigment should be in the range of 0.2-0.8. If the absorbance is outside this range then dilute the solution until the absorbance is within this range.

TABLE 5
List of standards in the standard mix or as single standards*

| | | | | | We have: |
|---|---|---|---|---|---|
| | | | | | Solution |
| | | | | λ HPLC | (6.6.1) |
| | | | | (nm) | Powder |
| Name | λₘₐₓ | E_{1 cm} | Solvent | (fra Uyi) | (6.6.2) |
| Chlorofyll c3 | 452.9 | 346.00 | 1 | 440 | 6.6.1 |
| Chlorofyll c2 | 443.8 | 347.00 | 1 | 440 | 6.6.1 |
| Peridin (Peridinin?) | 472.0 | 132.50 | 2 | 440 | 6.6.1 |
| Fucoxanthin | 449.0 | 160.00 | 2 | 440 | 6.6.1 |
| Neoxanthin | 439.0 | 224.30 | 2 | 440 | 6.6.1 |
| Prasinaxanthin | 454.0 | 160.00 | 2 | 440 | 6.6.1 |
| Violaxanthin | 443.0 | 255.00 | 2 | 440 | 6.6.1 |
| 19-hex-fuco | 447.0 | 160.00 | 2 | 440 | 6.6.1 |
| Astaxanthin | 477 | 217.39 | 1 | 440 | 6.6.2 |
| Antheraxanthin | 446 | 235 | 2 | 440 | 6.6.1 |
| Alloxanthin | 453.0 | 262.00 | 2 | 440 | 6.6.1 |
| Zeaxanthin | 450.0 | 254.00 | 2 | 440 | 6.6.1 |
| Lutein | 445.0 | 255.00 | 2 | 440 | 6.6.1 |
| Canthaxanthin | 476.0 | 207.50 | 2 | 440 | 6.6.2 |
| Chlorofyll b | 646.8 | 51.36 | 1 | 440 | 6.6.1 |
| Chlorofyll a** | 664 | 127 | 1 | 440 | 6.6.1 |
| Pheophytin | 667 | 51.2 | 1 | 410 | 6.6.1 |
| Alpha + | 453.0 | 262.00 | 2 | 440 | 6.6.1 |
| beta-carotene | | | | | (only beta) |
| Diadinoxanthin | 446.0 | 262.00 | 2 | 440 | 6.6.1 |
| Echinone | 458 | 215.8 | 2 | 440 | 6.6.1 |
| Pheophorbidea | 667 | 74.20 | 1 | 440 | 6.6.1 |
| Lycopene | 474 | 344.6 | 1 | 440 | 6.6.2 |
| MG DVP | | | | | |
| DV chlorofyll b | | | | | |
| DV chlorofyll a | 664.0 | 87.67 | 1 | | |
| Phytin a | 667.0 | 51.20 | 1 | | |

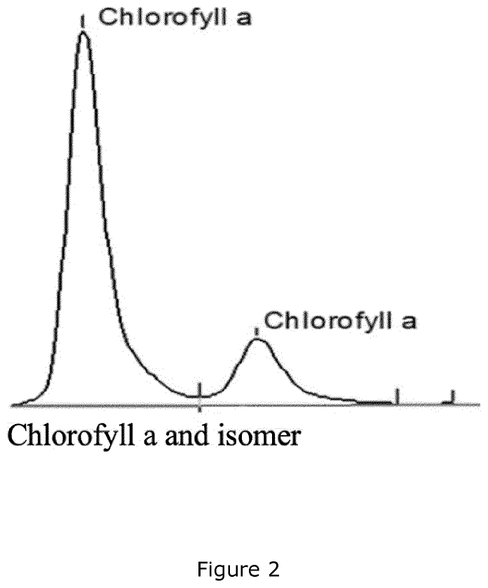

*Important: The standard mix from DHI can change from batch to batch. A chromatogram from DHI accompanies the standard mix. The chromatogram should be compared with a chromatogram like that shown in FIG. 2 when identifying the peaks. The two isomers can be seen when analyzing the standard using DAD.
Solvent 1 = 90% acetone
Solvent 2 = Ethanol
See FIG. 2

Concentrations are calculated as

$${C}_{STD}^{i} = {10}^{3} ( {A}^{i} ( {λ}_{max}^{i} ) - {A}^{i} ( 750 ) ) / b {E}_{1 cm}$$

$${C}_{STD}^{i} = concentration ( µg / ml ) of the standard for pigment i$$

$$( {A}^{i} ( {λ}_{max}^{i} ) = absorbances at λ max$$

$${A}^{i} ( 750 ) = absorbances at 750 nm$$

$$b = path length of the cuvette ( cm ) ( = 1 cm )$$

$${E}_{1 cm} = weight - specific extinction coefficient ( L g - 1 \cdot cm - 1 ) of pigment i .$$

### Preparation of Calibration Curve

The calibration curve is only made once per year, or when using a new pigment, which was not used in the previous calibration curve.
8.11 Calibration standards (either as mix or single standards) are prepared by diluting the DHI pigment standards (parent solution) with acetone+methanol (7+3) (6.8) with use of pipettes. For none-DHI standards, use the parent solution prepared in 8.9 and dilute it in acetone+methanol (7+3) (see 11.4). It is prudent to always do multipoint calibrations. Therefore, prepare at least 4 dilutions (e.g. no dilution, 4×, 8×, 16×) of the parent solution. After diluting you should end up with 100 μl. Calibration curves should be made in duplicates.
8.12 Determine the peak area by HPLC, using the method described below and make the calibration curve as peak area vs concentration. Make a calibration curve for each pigment. Response factor will be calculated as the slope of the regression of the peak areas of the parent pigment against the pigment concentrations of the injected working standards (μg/ml). To calculate the calibration curve, the peak areas and concentrations are entered into the Excel file “Pigment worksheet”. Calibration curves are obtained with coefficient of determination (r2) values never less than 0.99. Concentrations used for the calibration curves are usually within the range 0.01 to 1.5 μg/ml. In this range, the calibration curve is linear. If the sample concentration is outside the concentration range of the calibration curve, it should be diluted with acetone+methanol (6.8).

### HPLC Analysis

### Mobile Phase:

A: 70% Methanol+30% of 0.028 M TBAA in water (6.9)
B: Methanol (6.1)

### Method:

“Pigmentadvanced” method contains the following information:
Injection Volume: 100 μl (Use the Injection program)
Injection Program:
   Draw 28 Microliter from sample
   Draw 72 Microliter from Buffer (6.10) from position 91
   Mix in seat
   Wait 3 min
   Inject
Flow Rate: 0.9 ml/min

Gradient Program:

| Time(Min) | % of B |
|---|---|
| 0 | 5 |
| 27 | 95 |
| 34 | 95 |
| 35 | 100 |
| 38 | 100 |
| 40 | 5 |
| 46 | 5 |

Oven Temp.: 60° C.
Autosampler temp.: 4° C.
8.13 Select the method “Pigment advanced” under “Methods” in Open Lab.
8.14 Place a vial with the extraction solvent (6.8) position 1.
8.15 Place a vial with the pigment mix (6.7) in position 2. (see 11.3)
8.16 Place a vial with buffer (6.10) in position 91

### 9. Calculation

### Calculation of Concentration of Each Individual Peak of Pigment in a Sample

Enter the peak areas of the identified peaks, the weight of the sample and the solvent volume into the Excel file “Pigment worksheet”. The pigment concentrations are calculated from the calibration curves by:

Cpi=(Api−intersection)/slope×Vx/Mf

Cpi=concentration of pigment pi in sample (μg/g)
Api=peak area of pigment pi
Vx=extraction volume (in ml)
Mf=weight of sample (in g)

### 10. Accuracy of Analysis

Standard deviation (1 sigma) of the triplicates should <15%.

Other validation parameters which shall be determined in every lab:
Precision
LOD & LOQ
Recovery

### 11. Notes

11.1 The taxonomic composition of phytoplankton influences many biogeochemical processes, so it is essential to simultaneously determine phytoplankton biomass and composition over the continuum of phytoplankton size (approximately 0.5-100 Am). The determination of chlorophyll and carotenoid pigment concentrations by high-performance liquid chromatography (HPLC) is a i.e. the concentration of Chlorophyll a ost of these requirements. Indeed, many carotenoids and chlorophylls are taxonomic markers of phytoplankton taxa, which means community composition can be evaluated at the same time that [Chl a] i.e. the concentration of Chlorophyll a is accurately quantified. The possibility of determining community composition and biomass has resulted in the HPLC method rapidly becoming the technique of choice in biogeochemical and primary production studies. The use of HPLC methods in marine studies has also been promoted, because the international Joint Global Ocean Flux Study (JGOFS) program recommended HPLC in the determination of [Chl a] i.e. the concentration of Chlorophyll a and, more precisely, to use the protocol of Wright et al. (1991). Since the start of the JGOFS decade in the 1980s, HPLC techniques have evolved considerably (Jeffrey et al., 1999), and some JGOFS contributors decided not to follow the original JGOFS recommendation in order to take full benefit of the ongoing methodological evolutions. In particular, the C8 method of Goericke and Repeta (1993) was an important improvement, because it allowed the separation of divinyl chlorophyll a from its monovinyl form. Subsequent adaptations of this method were proposed (e.g., Vidussi et al., 1996; Barlow et al., 1997) and used for a variety of JGOFS cruises. More recently, new methods have also been proposed that rely on C8 phase and elevated column temperature to achieve the desired separation selectivity (Van Heukelem and Thomas, 2001) or on mobile phase modified with pyridine to resolve chlorophyll c pigments (Zapata et al., 2000).
   This protocol is a modified version of the Van Heukelem and Thomas (2001) method.
11.2 Analysis on wet and dried microalgae (Nannochloropsis salina and chlorella pyrenoidosa) biomasses has been compared and for Nannochloropsis there was no difference in the composition and content of the different pigments, whereas there was a difference in Chlorella for the chlorophylls. Both flash and freeze drying resulted in lower levels of chlorophylls compared to non-dried biomass, but there was no difference between the two drying methods. Due to easiness of handling, dried samples are preferred.
11.3 The standard mix is run as number 2 in the sequence. Then you can place the vial again in the freezer and use it again for subsequent runs.

In a more preferred embodiment the carotenoids and pigments are measured as follows: between 10-100 mg of dried microalgal powder was weighed off and mixed with 850 mg glass beads in a tube. A mixture of acetone and methanol was added. The mixture was vortexed for less than 1 minute and centrifuged 10-20 min at 1500 rpm. Subsequently the solvent layer was transferred to a new tube and the extraction was repeated two more times on the remaining pellet. All solvent fractions were combined and filtered through a 0.22 μm filter.

The extracts were analyzed using HPLC with 70% Methanol+30% 0.028 M Tetrabutyl ammonium acetate (TBAA) in water and pure methanol was mobile phases. Pigment extracts were diluted in a buffer containing TBAA solution in water and methanol. Separation took place over 45 min with a gradient of methanol from 5% to 95% over 27 min, 1 min to 100%, 3 min at 100%, 2 min to 5%, 6 min at 5%. Quantification of the pigments was performed by measuring certified pigments of known concentration and comparing the samples to this.

### Example 6: Photoautotrophic Cultivation of the Modified Strain of C. sorokiniana (M12) Compared to the Wild Type

### Purpose

In order to determine whether or not the modified strain of C. sorokiniana (M12) is able to grow photoautotrophic, the modified strain of C. sorokiniana (M12) and its wild type were cultivated at photoautotrophic and heterotrophic conditions on Solid Medium.

### Method

Equal amounts (approx.) of biomass from the modified strain of C. sorokiniana (M12) and its wild type (C. sorokiniana UTEX 1230) was plated on solid heterotrophic medium (comprising the same components as the Solid Medium described in Example 1, hereafter referred to as “Solid Heterotrophic Medium”) each with two replicates. The biomass on Solid Heterotrophic Medium was incubated at 28° C. for 10 days in complete darkness.

Equal amounts (approx.) of biomass from the modified strain of C. sorokiniana (M12) and its wild type (C. sorokiniana UTEX 1230) was also plated on solid photoautotrophic medium (comprising KNO₃, KH₂PO₄, MgSO₄·7H₂O, CaCl₂), citric acid, FeSO₄·7H₂O, Na₂EDTA, Arnon's A5 and agar, hereafter referred to as “Solid Photoautotrophic Medium”), each with two replicates. The biomass on Solid Photoautotrophic Medium was incubated at 23° C. for 10 days with an average light intensity of 55 μmol s⁻¹ m⁻².

The growth of the biomass was evaluated by comparing the plates with biomass at the day of inoculation of the solid media and after 10 days of incubation, using photography to document and compare.

### Result

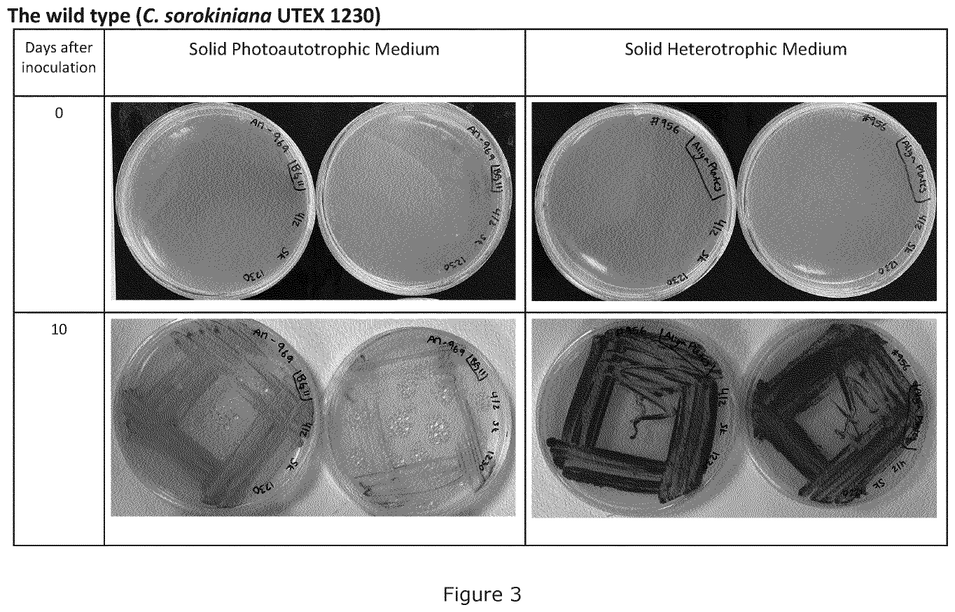

After 10 days of incubation, the biomass of the wild type (C. sorokiniana UTEX 1230) had grown on Solid Heterotrophic Medium as well as on Solid Photoautotrophic Medium (FIG. 3), verifying that the wild type is able to grow heterotrophic and photoautotrophic under the conditions set in the experiment.

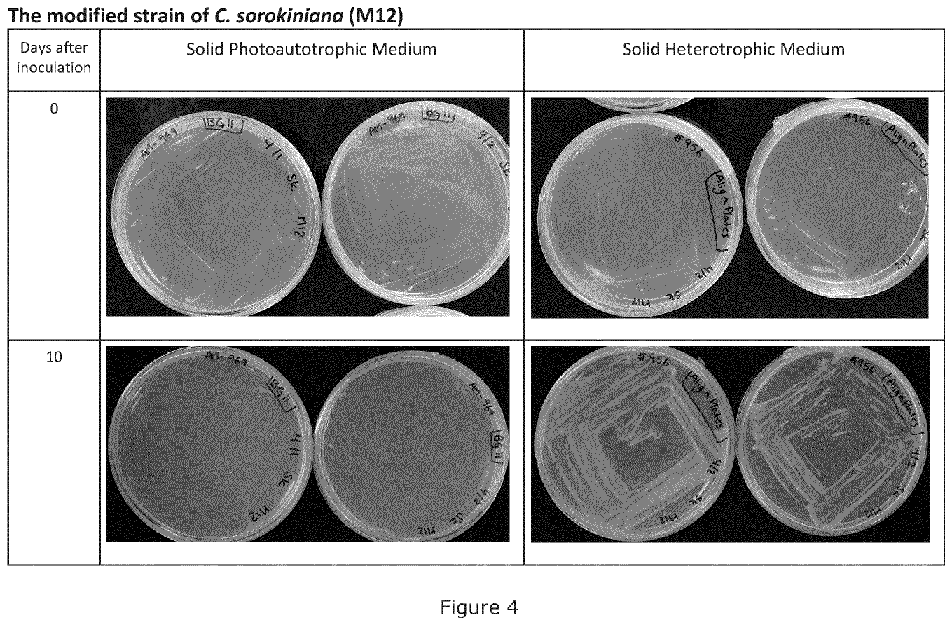

The experiment also showed that after 10 days of incubation, the modified strain of C. sorokiniana (M12) was able to grow on Solid Heterotrophic Medium but not on Solid Photoautotrophic Medium (FIG. 4). The results showed that the modified strain of C. sorokiniana is still capable of growing heterotrophically using glucose as a carbon source and urea as a nitrogen source like the wild type strain, but the mutations in the modified strain of C. sorokiniana have impaired the strain's ability to grow photoautotrophic.

1. A strain and/or a modified strain of C. sorokiniana having:
a chlorophyll content at or below 11 mg/g dry cell weight,
a lutein content below 1.5 mg/g dry cell weight,
a β-carotene content below 0.33 mg/g dry cell weight, and/or
a Neoxanthin content below 0.16 mg/g dry cell weight.

2. The strain and/or the modified strain of C. sorokiniana according to claim 1, wherein chlorophyll is chlorophyll a (a-chlorophyll) and/or chlorophyll b (b-chlorophyll).

3. The strain and/or the modified strain of C. sorokiniana according to claim 1, wherein the strain and/or the modified strain has a protein content of at least 20%, 25%, 30%, 35%, 40%, 45%, 55%, 60% or 65% w/w dry cell weight.

4. The strain and/or the modified strain of C. sorokiniana according to claim 1, wherein the strain has one or more mutations located in a gene upstream or downstream of a gene associated with:
Photosystem I and photosystem II,
Chloroplast ATP synthase,
Chloroplast Clp protease, or
Transfer RNA.

5. The strain and/or the modified strain of C. sorokiniana according to claim 1, wherein the strain has one or more mutations located in a gene upstream or downstream of one of the genes comprised in the chloroplast genome selected from the group consisting of:
psbM encoding the protein PSII reaction center protein M
trnS-UAG encoding t-RNA
psbI encoding the protein PSII reaction center protein I
ycf3 encoding PSI reaction center protein Ycf3
atpH encoding ATP synthase subunit c
psaB encoding PSI P700 chlorophyll a apoprotein A2
clpP encoding ATP-dependent Clp protease proteolytic subunit
and combinations thereof.

6. The strain and/or the modified strain of C. sorokiniana according to claim 4, wherein the one or more mutations are located in
(a) atpH and at a position corresponding to position 34544-34554 in SEQ ID NO 1.

7. The strain and/or the modified strain of C. sorokiniana according to claim 4, wherein the one or more mutations(s) are located upstream of:
(a) psbM and at a position according to 2992 in SEQ ID NO 1,
(b) psbI and at a position according to 29466 in SEQ ID NO 1,
(c) ycf3 and at a position according to 30075-30076 in SEQ ID NO 1, and/or
(d) clp and at a position according to 107103 in SEQ ID NO 1.

8. The strain and/or the modified strain of C. sorokiniana according to claim 4, wherein the one or more mutations are located downstream of:
a) trnS-UAG and at a position according to 2992 in SEQ ID NO 1, and/or
b) psaB and at a position according to 72653 in SEQ ID NO 1.

9. The strain and/or the modified strain of C. sorokiniana according to claim 4, wherein the one or more mutations are:
(a) a substitution from G to A at a position corresponding to position 2992 in SEQ ID NO 1,
(b) a substitution from T to A at a position corresponding to position 29466 in SEQ ID NO 1,
(c) a deletion of AA at a position corresponding to position 30075-30076 in SEQ ID NO 1,
(d) a deletion of CAGAAGCAGAA at a position corresponding to position 34544-34554 in SEQ ID NO 1,
(e) a deletion of A at a position corresponding to position 72653 in SEQ ID NO 1 and/or
(f) a deletion of A at a position corresponding to position 107103 in SEQ ID NO 1.

10. The strain and/or the modified strain of C. sorokiniana according to claim 1, wherein the strain and/or the modified strain of C. sorokiniana has the mutations of the C. sorokiniana M12 strain (deposit number CCAP 211/136).

11. The strain and/or the modified strain of C. sorokiniana according to claim 1, wherein the strain and/or the modified strain of C. sorokiniana is the C. sorokiniana strain according to deposit number CCAP 211/136.

12. A method for producing a strain and/or a modified strain of C. sorokiniana, wherein said method comprises the steps of:
a) obtaining a parental strain of C. sorokiniana,
b) subjecting the parental strain of C. sorokiniana to mutagenesis to generate a mutated strain of C. sorokiniana,
c) cultivating the mutated strain of C. sorokiniana on a medium comprising nicotine, norflurazon and/or diphenylamine and
d) identifying colonies of the mutated strain of C. sorokiniana having a phenotype different from the parental strain of C. sorokiniana as the strain and/or the modified strain of C. sorokiniana, and
e) obtaining a strain and/or a modified strain of Chlorella sorokiniana having a chlorophyll content at or below 11 mg/g dry cell weight, a lutein content below 1.5 mg/g dry cell weight, a β-carotene content below 0.33 mg/g dry cell weight, and/or a Neoxanthin content below 0.16 mg/g dry cell weight.

13. (canceled)

14. The method of claim 12, wherein the mutagenesis is performed by exposure of the parental strain C. sorokiniana to a mutagenic chemical selected from the group consisting of ethyl methanesulfonate, N-methyl-N′-nitro-N-nitrosoguanidine, and combinations thereof.

15. A C. sorokiniana obtainable by the method according to claim 12.

16. A method for producing an algae biomass, said method comprising the steps of:
(a) culturing the strain and/or the modified strain of C. sorokiniana of claim 1 in a media under aerobic heterotrophic conditions and
(b) obtaining an algae biomass.

17. An algae biomass obtainable by the method according to claim 16.

18. An algae biomass comprising the modified C. sorokiniana according to claim 1.

19. (canceled)

20. A composition comprising the strain and/or the modified strain of C. sorokiniana according to claim 1.

21. A food, human nutraceutical preparation or formulation, animal feed, pharmaceutical composition, vaccine, cosmetic, personal care composition, personal care device, textile, dye, ink, or packaging material, comprising the composition of claim 20.

22. (canceled)

23. A method for increasing protein content of a food product and/or a feed product, the method comprising adding the strain and/or the modified strain of C. sorokiniana according to claim 1 to a food product and/or a feed product to increase protein content of the food product and/or feed product.
